(19) United States
(12) Patent Application Publication
(10) Pub. No.: US 2026/0274868 A1
(43) Pub. Date: Sep. 17, 2026

(54) COMPOUNDS AND METHODS USEFUL FOR STABILIZING PHENYLALANINE HYDROXYLASE MUTATIONS

(71) Applicant: Agios Pharmaceuticals, Inc., Cambridge, MA (US)

(72) Inventors: Lucrezia De Pascalis, Cambridge, MA (US); Peng Liu, Belmont, MA (US); Brian C. Shook, Oakmont, PA (US)

(21) Appl. No.: 19/556,112

(22) Filed: Mar. 4, 2026

Related U.S. Application Data
(60) Provisional application No. 63/767,233, filed on Mar. 5, 2025.

(51) Int. Cl. C07D 519/00; A61K 31/497; C07B 59/00

(52) U.S. Cl. CPC C07D 519/00; A61K 31/497; C07B 59/002

(57) ABSTRACT

The disclosure relates to compounds of Formula I, or tautomer thereof, or a pharmaceutically acceptable salt of the compound or the tautomer, wherein, R¹ and R² are defined herein, and pharmaceutical compositions comprising compounds of Formula I, or tautomer thereof, or a pharmaceutically acceptable salt of the compound or tautomer.

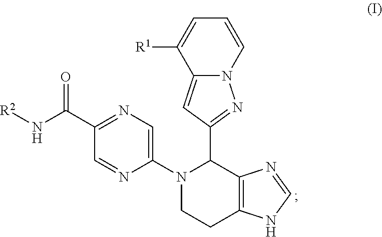

These compounds and pharmaceutical compositions are useful in methods for stabilizing a mutant PAH protein or reducing blood phenylalanine concentration in a subject suffering from phenylketonuria. In some embodiments, the mutant PAH protein contains at least one R408W, R261Q, R243Q, Y414C, L48S, A403V, I65T, R241C, L348V, R408Q, or V388M mutation. In other embodiments, the mutant PAH protein contains at least one R408W, Y414C, I65T, F39L, R408Q, L348V, R261Q, A300S, or L48S mutation.

### RELATED APPLICATIONS

This application claims priority to U.S. Provisional Patent Application No. 63/767,233, filed on Mar. 5, 2025. The entire contents of the foregoing application are expressly incorporated herein by reference.

### SEQUENCE LISTING

The present application contains a Sequence Listing XML file, which has been submitted electronically in .xml format as part of the specification, and is incorporated herein by reference in its entirety. Said XML file, created on Mar. 3, 2026, is named 128748-04702_SL.xml, and is 2,824 bytes in size.

### TECHNICAL FIELD

This application pertains to compounds that stabilize phenylalanine hydroxylase (PAH) mutations, pharmaceutical compositions comprising those compounds, and methods of using those compounds for treating phenylketonuria.

### BACKGROUND

Phenylketonuria (PKU) is an autosomal recessive disorder affecting approximately an average of 1:10,000 people worldwide (approx. 1:25,000 in the U.S.). The number of patients varies globally depending on region. PKU arises in patients who have mutations in the gene encoding the phenylalanine hydroxylase (PAH) enzyme responsible for converting phenylalanine to tyrosine. PAH is a tetrameric enzyme expressed in the liver requiring BH4 cofactor for activity. Reduction or loss of PAH activity results in toxic accumulation of phenylalanine (Phe) in the blood and brain. High levels of Phe damage brain white matter and interfere with neurotransmitter production. If untreated, high levels of Phe can result in mental retardation and decreased IQ in children and neurocognitive and psychiatric issues in adults, such as executive function deficits (for example, difficulty with attention, memory, flexible thinking, and organization/time management), psychological issues (for example, depression, anxiety, and mood swings), psychiatric and/or behavioral issues (for example, attention deficit hyperactivity disorder, self-harm, schizophrenia, agoraphobia, and agitation) and neurological abnormalities (for example, spasticity, tremor, gait disturbances, and seizures).

PKU is characterized by elevated blood Phe concentration or hyperphenylalaninemia (HPA). Normal blood Phe concentrations range from 50 to 110 μM. PKU phenotypes can vary from mild hyperphenylalaninemia (HPA) to more severe phenotypes that result in untreated blood Phe concentrations exceeding 1200 μM. American medical guidelines currently recommend maintaining blood Phe concentration in the range of 120 to 360 μM in both adults and children under the age of 12 years. European medical guidelines currently recommend maintaining blood Phe concentration below 360 μM in children under the age of 12 years and in pregnant women and below 600 μM in non-pregnant patients older than 12 years.

A standard of care for treating PKU is a Phe-restricted diet that severely limits the intake of natural protein, supplemented with medical foods to provide protein equivalents and prevent nutritional deficiencies. Such diets are very strict diets and challenging to adhere to. Three medications are currently approved for treating PKU. KUVAN® (sapropterin dihydrochloride s is a synthetic BH4 cofactor approved in 2007 for use in infants to adults. KUVAN® is not effective for all PKU patients, and the current guidelines suggest response testing in patients unless the patient is known to have two null mutations. SEPHIENCE™ (sepiapterin) was approved in 2025 for use in the treatment of hyperphenylalaninemia in adult and pediatric patients 1 month of age and older with sepiapterin-responsive PKU and is to be used with a Phe-restricted diet. Like KUVAN®, the current guidelines for SEPHIENCE™ recommend response testing in patients prior to treatment. Pegvaliase is an enzyme substitution therapy approved in 2018 for adults with a blood Phe concentration greater than 600 μM, despite prior management with available treatment options. Pegvaliase typically involves injection of a purified PEGylated form of phenylalanine ammonia lyase that reduces Phe by converting it to ammonia and trans-cinnamic acid instead of tyrosine. One of the main complications with enzyme substitution therapy is the attainment and maintenance of therapeutically effective amounts of protein in vivo due to rapid degradation or inactivation of the infused protein. A current approach to overcome this problem is to perform numerous costly high dose injections.

Pharmaceutical agents that enable patients to increase their intake of natural protein are desired.

### SUMMARY

In some aspects, the disclosure provides compounds of Formula I.

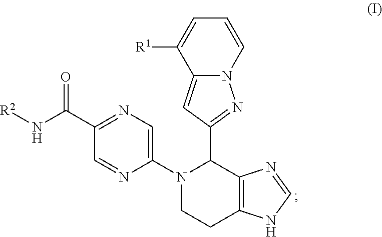

or a tautomer thereof, or a pharmaceutically acceptable salt of the compound or the tautomer, wherein: R¹ is hydrogen or fluoro; and R² is cyclopropyl, methyl, t-butyl, or deuterated(C₁-C₄)alkyl.

The compounds of Formula I, which contain a pyrazine-C(O)NH—R² moiety, have been found to show improvements in one or more drug-like properties including, e.g., ability to stabilize a mutant PAH protein, greater metabolic stability, greater cellular permeability, and greater oral bioavailability (which, for example, can enable a lower dose to be administered), less hERG inhibition potential, less aromatase inhibition potential, and less cytochrome P450 (CYP) inhibition potential. See, e.g., the Examples section below.

Stereoisomers and mixtures of stereoisomers of the compounds of Formula I, and the tautomers and pharmaceutical salts of the compound or the tautomers, are also described.

In further aspects, the disclosure provides pharmaceutical compositions comprising one or more compounds as described herein or a pharmaceutically acceptable salt of the compounds or the tautomers described herein; and a pharmaceutically acceptable excipient.

In yet other aspects, the disclosure provides methods for stabilizing a mutant PAH protein, comprising contacting the protein with one or more compounds as described herein or a pharmaceutically acceptable salt of the compounds or the tautomers described herein. In some embodiments, the mutant PAH protein contains at least one R408W, R261Q, R243Q, Y414C, L48S, A403V, I65T, R241C, L348V, R408Q, or V388M mutation. In other embodiments, the mutant PAH protein contains at least one R408W, Y414C, I65T, F39L, R408Q, L348V, R261Q, A300S, or L48S mutation.

In still further aspects, the disclosure provides methods for reducing phenylalanine levels in a subject suffering from phenylketonuria comprising administering a therapeutically effective amount of one or more compounds as described herein or a pharmaceutically acceptable salt of the compounds or the tautomers described herein.

### DETAILED DESCRIPTION OF ILLUSTRATIVE EMBODIMENTS

### 1. Definitions

Unless otherwise defined, all technical and scientific terms used herein have the same meaning as commonly understood by one of ordinary skill in the art to which this disclosure pertains. The terminology used in the description is for describing particular embodiments only and is not intended to be limiting of the disclosure.

The articles “a” and “an” as used herein and in the appended claims are used herein to refer to one or to more than one (e.g., to at least one) of the grammatical object of the article unless the context clearly indicates otherwise. By way of example, “an element” means one element or more than one element.

“Pharmaceutically acceptable” means approved or approvable by a regulatory agency of the Federal or a state government or the corresponding agency in a country other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic and may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, fumarate, tartrate, mesylate, acetate, maleate, oxalate, and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith.

The term “(C₁-C₄)alkyl,” refers to a straight- or branched-chain hydrocarbon group having from 1 to 4 carbon atoms in the group. Examples of (C₁-C₄)alkyl groups include methyl (C₁), ethyl (C₂), propyl (C₃) (e.g., n-propyl, isopropyl), and butyl (C₄) (e.g., n-butyl, tert-butyl, sec-butyl, iso-butyl).

The term “deuterated(C₁-C₄)alkyl,” refers to a (C₁-C₄)alkyl group, wherein one or more hydrogen atoms are replaced by deuterium.

When a range of carbon atoms is used herein, for example, C₁₋₄, all ranges, as well as individual numbers of carbon atoms are encompassed. For example, “C₁₋₄” includes C₁₋₄, C₁₋₃, C₁₋₂, C₂₋₃, C₂₋₄, C₃₋₄, C₁, C₂, C₃, and C₄.

Recitation of ranges of values herein are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “about” when used in combination with a numeric value or range of values means the value or range of values may deviate to an extent deemed reasonable to one of ordinary skill in the art.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including supercritical fluid chromatography (SFC), chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972).

As used herein, the term “stereoisomers” refers to compounds which have identical chemical constitution and connectivity but differ with regard to the arrangement of the atoms or groups in space, e.g., enantiomers or diastereomers.

When the stereochemical configuration at a chiral center in a compound having one or more chiral centers is depicted by its chemical name (e.g., where the configuration is indicated in the chemical name by “R” or “S”) or structure (e.g., the configuration is indicated by dashed or wedge bonds), the enrichment of the indicated configuration relative to the opposite configuration is greater than 50%, 60%, 70%, 80%, 90%, 99% or 99.9%. “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When a disclosed compound is named or depicted by structure without indicating stereochemistry, it is understood that the name or the structure encompasses one of the possible stereoisomers or geometric isomers free of the others, or a mixture of the encompassed stereoisomers or geometric isomers.

It will be understood that certain compounds disclosed herein may exist in tautomeric forms. Such forms are included as part of the present disclosure. Thus, when a compound herein is represented by a structural formula or designated by a chemical name herein, all tautomeric forms which may exist for the compound are encompassed by the structural formula.

When the compounds described herein contain olefinic double bonds or other centers of geometric asymmetry, and unless specified otherwise, it is intended that they include both E and Z geometric isomers.

With regard to the compounds provided herein, when a particular atom's position is designated as having deuterium or “D” or “²H”, it is understood that the abundance of deuterium at that position is substantially greater than the natural abundance of deuterium, which is about 0.015%. A position designated as having deuterium typically has a minimum isotopic enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 4000 (60% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium atom. The isotopic enrichment and isotopic enrichment factor of the compounds provided herein can be determined using conventional analytical methods known to one of ordinary skill in the art, including mass spectrometry and nuclear magnetic resonance spectroscopy.

The term “patient” or “subject” is used throughout the specification to describe an animal, preferably a human or a domesticated animal, to whom treatment, including prophylactic treatment, with the compounds or pharmaceutical compositions according to the present disclosure is provided. For treatment of those conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

The terms “therapeutically effective amount” or “effective amount” means an amount or dose of a compound of the disclosure (or a pharmaceutically acceptable salt thereof) sufficient to generally bring about the desired therapeutic benefit in subjects in need of such treatment for the designated disease or disorder. Further, a therapeutically effective amount with respect to a compound of the disclosure means that amount of therapeutic agent alone, or in combination with other therapies, that provides a therapeutic benefit in the treatment or prevention of a disease.

“Treating” or “treatment” of any disease or disorder refers, in one embodiment, to ameliorating the disease or disorder, (e.g., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

The terms “prevent,” “preventing,” and “prevention” refer to the prevention of the onset, recurrence, or spread of the disease in a subject resulting from the administration of a prophylactic or therapeutic agent.

### 2. Compounds

The present disclosure provides compounds of Formula I:

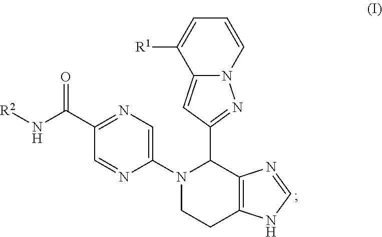

or a tautomer thereof, or a pharmaceutically acceptable salt of the compound or the tautomer, wherein R¹ is hydrogen or fluoro; and R² is cyclopropyl, methyl, t-butyl, or deuterated(C₁-C₄)alkyl.

In an aspect, the compounds of Formula I are of the Formula Ia:

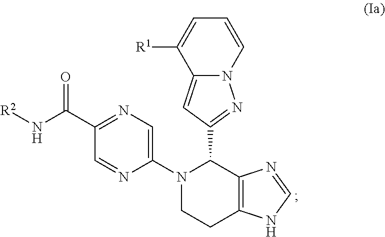

or a tautomer thereof, or a pharmaceutically acceptable salt of the compounds or the tautomer.

In an aspect, the compounds of Formula I are of the Formula Ib:

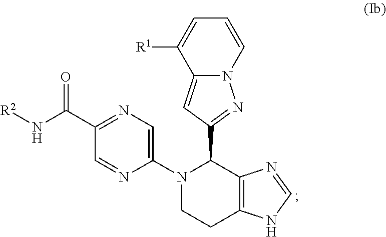

or a tautomer thereof, or a pharmaceutically acceptable salt of the compounds or the tautomer.

In an aspect, R¹ in the compounds of Formula I, Formula Ia, or Formula Ib, or a tautomer thereof, or a pharmaceutically acceptable salt of the compound or the tautomer, is hydrogen.

In an aspect, R¹ in the compounds of Formula I, Formula Ia, or Formula Ib, or a tautomer thereof, or a pharmaceutically acceptable salt of the compound or the tautomer, is fluoro.

In an aspect, R² in the compounds of Formula I, Formula Ia, or Formula Ib, or a tautomer thereof, or a pharmaceutically acceptable salt of the compound or the tautomer, is cyclopropyl.

In an aspect, R² in the compounds of Formula I, Formula Ia, or Formula Ib, or a tautomer thereof, or a pharmaceutically acceptable salt of the compound or the tautomer, is methyl.

In an aspect, R² in the compounds of Formula I, Formula Ia, or Formula Ib, or a tautomer thereof, or a pharmaceutically acceptable salt of the compound or the tautomer, is t-butyl.

In an aspect, R² in the compounds of Formula I, Formula Ia, or Formula Ib, or a tautomer thereof, or a pharmaceutically acceptable salt of the compound or the tautomer, is deuterated(C₁-C₄)alkyl.

In an aspect, R² in the compounds of Formula I, Formula Ia, or Formula Ib, or a tautomer thereof, or a pharmaceutically acceptable salt of the compound or the tautomer, is —CD₃, —C(CD₃)₃, —CD₂CD₃, or —CD(CD₃)₂.

In an aspect, the compounds of Formula I or Ia have improvements in metabolic stability. In an aspect, the compounds of Formula I or Ia are low clearance compounds as demonstrated by the compound's hepatic extraction ratio (Eₕ) in human liver microsomes.

In an aspect, the compounds of Formula I or Ia have improvements in cellular permeability and/or oral bioavailability.

In an aspect, the compounds of Formula I or Ia have improvements in decreased aromatase inhibition potential. Aromatase (CYP19A1) is a cytochrome P450 enzyme that catalyzes a critical step in the conversion of androgens (male hormones) to estrogens (female hormones).

In an aspect, the compounds of Formula I or Ia have improvements in decreased cardiac hERG potassium ion channel inhibition potential. Inhibition of hERG can result in the lengthening of a patient's cardiac QT interval, leading to the risk of potentially fatal cardiac arrhythmias.

In an aspect, the compounds of Formula I or Ia have improvements in decreased inhibition of Cytochrome P450 (CYP) isoforms. Sufficient inhibition of a particular CYP isoform may result in problematic drug-drug interactions (DDIs) in patients taking additional medications in addition to the compounds described herein. In an aspect, the CYP isoform is one or more of CYP1A2, CYP2C19, CYP2C9, CYP2D6, or CYP3A4.

Compounds having the Formula I, Formula Ia, or Formula Ib, are further disclosed in the Exemplification section and are included in the present disclosure. Tautomers, pharmaceutically acceptable salts of the compounds and tautomers thereof as well as the neutral forms are included.

The disclosure further provides R-enantiomers, S-enantiomers, or racemic mixtures of any of the compounds described herein. In an aspect, the compound is an S-enantiomer. In an aspect, the compound is the R-enantiomer. In an aspect, the compound is racemic.

In an aspect, the compounds of the disclosure may be enantiomerically enriched, e.g., the enantiomeric excess or “ee” of the compound is greater than about 5% as measured by chiral HPLC. In an aspect, the ee is greater than about 10%. In an aspect, the ee is greater than about 20%. In an aspect, the ee is greater than about 30%. In an aspect, the ee is greater than about 40%. In an aspect, the ee is greater than about 50%. In an aspect, the ee is greater than about 60%. In an aspect, the ee is greater than about 70%. In an aspect, the ee is greater than about 80%. In an aspect, the ee is greater than about 85%. In an aspect, the ee is greater than about 90%. In an aspect, the ee is greater than about 91%. In an aspect, the ee is greater than about 92%. In an aspect, the ee is greater than about 93%. In an aspect, the ee is greater than about 94%. In an aspect, the ee is greater than about 95%. In an aspect, the ee is greater than about 96%. In an aspect, the ee is greater than about 97%. In an aspect, the ee is greater than about 98%. In an aspect, the ee is greater than about 99%.

The present disclosure encompasses the preparation and use of salts of compounds of the disclosure. Salts of compounds of the disclosure can be prepared during the final isolation and purification of the compounds or separately by reacting the compound with an acid or base as appropriate.

### 3. Treatment Methods

Compounds and pharmaceutical compositions of the disclosure have several uses as described herein. In some embodiments, compounds and pharmaceutical compositions of the disclosure are useful in methods for stabilizing mutant PAH proteins and methods for treating subjects with phenylketonuria. These methods comprise contacting the protein with one or more compounds described herein, or a pharmaceutically acceptable salt thereof, or a pharmaceutical composition described herein. The compounds and pharmaceutical compositions of the disclosure can provide for better Phe control for patients whose disease is not well-managed on diet alone and lessen the severity of a patient's phenylketonuria. Thus, patients administered a compound or pharmaceutical composition of the disclosure will have a better quality of life, e.g., a more normal lifestyle and/or none or fewer dietary restrictions, as compared with phenylketonuria patients who have not been administered a compound or pharmaceutical composition of the disclosure. In some embodiments, patients administered a compound or pharmaceutical composition of the disclosure may experience increases in executive function, decreases in anxiety symptoms, and/or decreases in attention deficit hyperactivity disorder symptoms.

The term “mutant PAH gene” as used herein refers to the full DNA sequence of PAH that differs in one or more ways from the canonically accepted sequence (“the basis gene”) that is published in any one of a variety of curated databases. As one example, the sequence described by GenBank Accession number NG_008690.2 describes the basis gene.

The term “mutant PAH protein” as used herein refers to a PAH protein that contains at least one mutation in the amino acid sequence relative to that encoded by the reference. The reference human PAH protein is described by Genbank Accession number NP_000268 and contains 452 amino acids. PAH protein mutations can be identified using methods known in the art. In some embodiments, the mutant PAH protein contains at least one R408W, R261Q, R243Q, Y414C, L48S, A403V, I65T, R241C, L348V, R408Q, or V388M mutation. In other embodiments, the mutant PAH protein contains at least one R408W, Y414C, I65T, F39L, R408Q, L348V, R261Q, A300S, or L48S mutation. In still other embodiments, the mutant PAH protein contains at least one R408W, R243Q, R408Q, V388M, or L348V mutation. In yet other embodiments, the mutant PAH protein contains at least one R408W mutation. In further embodiments, the mutant PAH protein contains at least two R408W mutations. In further embodiments, the mutant PAH protein contains at least one R261Q mutation. In yet other embodiments, the mutant PAH protein contains at least one R243Q mutation. In yet other embodiments, the mutant PAH protein contains at least one Y414C mutation. In still further embodiments, the mutant PAH protein contains at least one L48S mutation. In other embodiments, the mutant PAH protein contains at least one A403V mutation. In further embodiments, the mutant PAH protein contains at least one I65T mutation. In yet further embodiments, the mutant PAH protein contains at least one R241C mutation. In yet other embodiments, the mutant PAH protein contains at least one L348V mutation. In further embodiments, the mutant PAH protein contains at least one R408Q mutation. In other embodiments, the mutant PAH protein contains at least one V388M mutation. In other embodiments, the mutant PAH protein contains at least one F39L mutation. In still further embodiments, the mutant PAH protein contains at least one A300S mutation. In yet further embodiments, the mutant PAH protein contains at least one L48S mutation.

In other embodiments, the disclosure provides methods for stabilizing the activity of mutant phenylalanine hydroxylase (PAH) proteins as compared to wild type PAH. Such methods include contacting phenylalanine hydroxylase with one or more compounds described herein, or a pharmaceutically acceptable salt thereof, or a pharmaceutical composition described herein. The term “stabilizing” as used herein refers to modulating the activity or quantity of a PAH enzyme so that it catalyzes hydroxylation of the aromatic side-chain of phenylalanine at a rate that is more similar to the PAH catalysis rate of a control population having wild type PAH, i.e., without a mutant PAH gene mutation, as compared to the baseline PAH catalysis rate. In some aspects, the term “stabilizing” refers to modulating the activity of a subject's PAH so that it catalyzes hydroxylation of the aromatic side-chain of phenylalanine at a flux more similar to the PAH catalytic flux of a control subject population without a mutant PAH gene mutation. In some embodiments, “stabilizing” activity of PAH includes increasing levels of the enzyme PAH as compared to baseline. By increasing the buildup of stabilized active PAH protein, a subject's toxic Phe levels can be reduced as compared to the subject's baseline levels of dietary Phe prior to administration of a compound of the disclosure or a pharmaceutical composition comprising compounds of the disclosure.

In some embodiments, the disclosure provides methods for reducing blood phenylalanine concentrations in a subject suffering from phenylketonuria to a concentration less than or equal to about 600 μM. In other embodiments, the blood Phe concentration is reduced to a concentration less than or equal to about 360 μM. In other embodiments, the disclosure provides methods for reducing blood Phe concentrations as compared to untreated baseline in a subject. In some embodiments, a subject's blood Phe concentration as compared to untreated baseline is reduced by a percentage including but not limited at least about 10%, at least about 20%, at least about 30%, at least about 40%, at least about 50%, at least about 60%, at least about 70%, at least about 80%, or at least about 90%. In other embodiments, a subject's blood Phe concentration as compared to untreated baseline is reduced by at least about 10%. In further embodiments, a subject's blood Phe concentration as compared to untreated baseline is reduced by at least about 20%. In yet other embodiments, a subject's blood Phe concentration as compared to untreated baseline is reduced by at least about 30%. In still further embodiments, a subject's blood Phe concentration as compared to untreated baseline is reduced by at least about 40%. In other embodiments, a subject's blood Phe concentration as compared to untreated baseline is reduced by at least about 50%. In further embodiments, a subject's blood Phe concentration as compared to untreated baseline is reduced by at least about 60%. In yet other embodiments, a subject's blood Phe concentration as compared to untreated baseline is reduced by at least about 70%. In still further embodiments, a subject's blood Phe concentration as compared to untreated baseline is reduced by at least about 80%. In other embodiments, a subject's blood Phe concentration as compared to untreated baseline is reduced by at least about 90%. A subject's Phe concentration can be determined by blood tests and methods for measuring such levels are known in the art. In some embodiments, the reduction in Phe concentration achieved using compounds of the disclosure is obtained in conjunction with the subject actively managing their dietary Phe intake. In other embodiments, the reduction in Phe concentration is obtained in conjunction with the subject maintaining a Phe-restricted diet.

In some embodiments, a subject is treated with a compound of the disclosure, or a pharmaceutically acceptable salt thereof, or a pharmaceutical composition comprising a compound of the disclosure, or a pharmaceutically acceptable salt thereof. The compound, or a pharmaceutically acceptable salt thereof, or pharmaceutical composition is administered in an amount sufficient for stabilizing the PAH protein, or for reducing blood phenylalanine concentration in a subject, or combinations thereof in the subject.

In further embodiments, the subject is a human patient, such as a human adult over 18 years old in need of treatment. In other embodiments, the human patient is a patient over 16 years old in need of treatment. In yet other embodiments, the human patient is a patient over 12 years old in need of treatment. In yet further embodiments, the human patient is a human child less than 18 years old. In still further embodiments, the human patient is a human child between 12 years and 18 years old. In yet other embodiments, the human patient is a human child less than 12 years old. In any of the embodiments, the subject has phenylketonuria (PKU), optionally classic PKU or severe PKU. In some embodiments, the subject has hyperphenylalaninemia (HPA). In some embodiments, the subject has a blood Phe concentration greater than about 600 μM prior to administration of a compound of the disclosure or a pharmaceutical composition comprising compounds of the disclosure. In other embodiments, the subject's blood Phe concentration prior to administration is greater than about 700 μM. In further embodiments, the subject's blood Phe concentration prior to administration is greater than about 800 μM. In still further embodiments, the subject's blood Phe concentration prior to administration is greater than about 900 μM. In yet other embodiments, the subject's blood Phe concentration prior to administration is greater than about 1000 μM. In further embodiments, the subject's blood Phe concentration prior to administration is greater than about 1100 μM. In other embodiments, the subject's blood Phe concentration prior to administration is greater than about 1200 μM.

The present methods also encompass administering an additional therapeutic agent to the subject in addition to the compounds of the disclosure. In some embodiments, the additional therapeutic agent is selected from drugs known as useful in a stabilizing mutant PAH protein and/or reducing blood Phe concentrations. The additional therapeutic agent is different from the compounds of the disclosure. In some embodiments, the additional therapeutic agent is sapropterin, sapropterin dihydrochloride, or sepiapterin. In other embodiments, the additional therapeutic agent is another pharmacological chaperone known to be useful in stabilizing mutant PAH protein. In further embodiments, the additional therapeutic agent is PBAS499. In other embodiments, the additional therapeutic agent is a nutritional supplement. Nutritional supplements that may be used include those that contain amino acids and other nutrients. In further embodiments, the nutritional supplement contains large neutral amino acids such as leucine, tyrosine, tryptophan, methionine, histidine, isoleucine, valine, threonine. In other embodiments, the nutritional supplement contains tyrosine. In further embodiments, the nutritional supplement contains casein glycomacropeptide, i.e., a milk peptide naturally free of Phe in its pure form. In other embodiments, the additional therapeutic agent is an enzyme substrate or enzyme co-factor. In yet other embodiments, the enzyme substrate or co-factor is tetrahydrobiopterin. In other embodiments, the additional therapeutic agent is a biopterin analogue. In further embodiments, the additional therapeutic agent is a biotherapeutic, synthetic biotic, microbiota or probiotic. In yet other embodiments, the biotherapeutic, synthetic biotic, microbiota or probiotic contains a genetically modified phenylalanine ammonia lyase (PAL) gene, such as, for example, E. coli Nissle PAL. Examples of genetically modified E. coli Nissle PAL biotherapeutics include SYNB1934 and SYNB1618, and the like. In still further embodiments, the additional therapeutic agent is an inhibitor of an amino acid transporter. In some embodiments, the amino acid transporter is B⁰AT1 (also referred to as SLC6A19), and the additional therapeutic agent is a SLC6A19 inhibitor. Examples of SLC6A19 inhibitors include nimesulide, benztropine, NSC63912, NSC22789, cinromide, CB3, E62, JNT-517, and the like.

In embodiments, provided herein is a method for stabilizing a mutant PAH protein, comprising contacting the protein with a compound of the present disclosure, or a tautomer thereof, or a pharmaceutically acceptable salt of the compound or the tautomer, or a pharmaceutical composition of the present disclosure.

In some embodiments, the mutant PAH protein contains at least one R408W, R261Q, R243Q, Y414C, L48S, A403V, I65T, R241C, L348V, R408Q, or V388M mutation. In some embodiments, the mutant PAH protein contains at least one R408W, Y414C, I65T, F39L, R408Q, L348V, R261Q, A300S, or L48S mutation. In some embodiments, the mutant PAH protein contains at least one R408W mutation. In some embodiments, the mutant PAH protein contains two R408W mutations.

In embodiments, provided herein is a method for reducing blood phenylalanine concentration in a subject suffering from phenylketonuria, comprising administering a compound of the present disclosure, or a tautomer thereof, or a pharmaceutically acceptable salt of the compound or the tautomer, or a pharmaceutical composition of the present disclosure.

In some embodiments, the blood phenylalanine concentration is reduced to a concentration less than or equal to about 600 μM. In some embodiments, the blood phenylalanine concentration is reduced to a concentration less than or equal to about 360 μM. In some embodiments, the subject has a blood phenylalanine concentration greater than about 600 μM prior to administration of the compound. In some embodiments, the subject has a blood phenylalanine concentration greater than about 1200 μM prior to administration of the compound.

Compounds of the disclosure and the additional therapeutic agents can be administered simultaneously or sequentially to achieve the desired effect. In addition, the compounds of the disclosure and additional therapeutic agent can be administered in a single pharmaceutical composition or two separate pharmaceutical compositions.

The additional therapeutic agent is administered in an amount to provide its desired therapeutic effect. The effective dosage range for each additional therapeutic agent is known in the art, and the additional therapeutic agent is administered to an individual in need thereof within such established ranges.

Compounds and pharmaceutical compositions of the disclosure and the additional therapeutic agents can be administered together as a single-unit dose or separately as multi-unit doses, wherein the compounds or pharmaceutical compositions of the disclosure are administered before the additional therapeutic agent or vice versa. One or more doses of the compounds or pharmaceutical compositions of the disclosure and/or one or more dose of the additional therapeutic agents can be administered.

The compounds and pharmaceutical compositions of the disclosure may also be administered sequentially or concurrently with non-pharmacological techniques. In some embodiments, the patient uses non-pharmacological techniques to maintain lower Phe levels. In other embodiments, the non-pharmacological technique is administering a diet that is low in Phe. One skilled in the art would be able to determine what type of diet to maintain appropriate levels of Phe. In some embodiments, a Phe-restricted diet is a diet containing about 200 to about 500 mg/day (patients 10 years or younger) of Phe or less than about 600 mg/day (patients over 10 years of age). In other embodiments, the diet may include restricting or eliminating one or more foods that are high in Phe, such as soybeans, egg whites, shrimp, chicken breast, spirulina, watercress, fish, nuts, crayfish, lobster, tuna, turkey, legumes, and low-fat cottage cheese.

An example of a dose of a compound disclosed herein is in the range of from about 0.001 to about 100 mg of compound per kg of subject's body weight per day, in single or divided dosage units (e.g., BID, TID, QID). For a 70-kg human, a suitable dosage amount is from about 0.05 to about 7 g/day.

In some embodiments, the therapeutically effective amount of one or more compounds or pharmaceutical compositions described herein is an amount that is effective in stabilizing a mutant PAH protein described herein. In other embodiments, the therapeutically effective amount of one or more compounds or pharmaceutical compositions described herein is an amount that is effective in reducing blood phenylalanine concentrations.

Unless otherwise noted, the amounts of the compounds described herein are set forth on a free base basis. That is, the amounts indicate that amount of the compound administered, exclusive of, for example, solvent or counterions (such as in pharmaceutically acceptable salts).

### 4. Pharmaceutical Compositions

The disclosure also provides pharmaceutical compositions comprising compounds of the disclosure, or a pharmaceutically acceptable salt thereof, and a pharmaceutically acceptable carrier or excipient.

The methods of the present disclosure can be accomplished by administering compounds of the disclosure as the neat compound or as a pharmaceutical composition. Administration of a pharmaceutical composition, or neat compound of the disclosure, can be performed at any time period as determined by the attending physician. Typically, the pharmaceutical compositions contain no toxic, carcinogenic, or mutagenic compounds that would cause an adverse reaction when administered.

Pharmaceutical compositions include those wherein compounds of the disclosure are administered in an effective amount to achieve its intended purpose. The exact formulation, route of administration, and dosage is determined by an individual physician.

Compounds of the disclosure can be administered by any suitable route, e.g., by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high-pressure technique.

The above-mentioned additional therapeutically active agents, one or more of which can be used in combination with compounds of the disclosure, are prepared and administered as described in the art.

Compounds of the disclosure may be administered in admixture with pharmaceutical carriers selected with regard to the intended route of administration and standard pharmaceutical practice. Pharmaceutical compositions for use in accordance with the present disclosure are formulated in a conventional manner using one or more physiologically acceptable carriers comprising excipients and/or auxiliaries that facilitate processing of compounds of the disclosure.

Administration of the compounds or pharmaceutical compositions of the disclosure can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. Compounds can also be administered intraadiposally or intrathecally.

The amount of the compound or pharmaceutical composition administered will be dependent on the subject being treated, the severity of the disorder or condition, the rate of administration, the disposition of the compound and the discretion of the prescribing physician. The desired dose can be administered in a single dose, or as multiple doses administered at appropriate intervals, e.g., as one, two, three, four or more subdoses per day. In some embodiments, the compounds and pharmaceutical compositions disclosed herein are effective over a wide dosage range. For example, in the treatment of adult humans, dosage forms containing from about 0.01 to 2000 mg of a compound disclosed herein per day are examples of dosage forms that may be used. The exact dosage will depend upon the route of administration, the form in which the compound is administered, the subject to be treated, the body weight of the subject to be treated, and the preference and experience of the attending physician. In some instances, dosage levels below the lower limit of the aforesaid range may be more than adequate, while in other cases still larger doses may be employed without causing any harmful side effect, e.g., by dividing such larger doses into several small doses for administration throughout the day.

In some embodiments, a compound of the disclosure is administered in a single dose.

Typically, such administration will be by a solid oral dosage form such as tablet or capsule. However, other routes may be used as appropriate. A single dose of a compound may also be used for treatment of an acute condition.

In some embodiments, a compound of the disclosure may be administered in multiple doses. Dosing may be about once, twice, three times, four times, five times, six times, or more than six times per day. In another embodiment, a compound described herein and another therapeutic agent are administered together about once per day to about 6 times per day.

Administration of the compounds disclosed herein may continue as long as necessary. In some embodiments, a compound is administered chronically on an ongoing basis, e.g., for the treatment of chronic effects.

An effective amount of a compound or pharmaceutical composition of the disclosure may be administered in either single or multiple doses by any of the accepted modes of administration of therapeutic agents having similar utilities, including rectal, buccal, intranasal and transdermal routes, by intra-arterial injection, intravenously, intraperitoneally, parenterally, intramuscularly, subcutaneously, orally, topically, or as an inhalant.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include one or more conventional pharmaceutical carriers or excipients and a compound disclosed herein as an active ingredient. In addition, it may include other medicinal or pharmaceutical agents, carriers, adjuvants, etc.

Exemplary parenteral administration forms include solutions or suspensions of the compound of the disclosure in sterile aqueous solutions, for example, aqueous propylene glycol or dextrose solutions. Such dosage forms can be suitably buffered, if desired.

### 5. Pharmaceutical Compositions for Oral Administration

In some embodiments, the disclosure provides a pharmaceutical composition for oral administration containing a compound of the disclosure, or a pharmaceutically acceptable salt thereof, and pharmaceutical excipients suitable for oral administration.

In some embodiments, the disclosure provides a solid pharmaceutical composition for oral administration containing: (i) an effective amount of a compound of the disclosure; optionally (ii) an effective amount of a second therapeutic agent; and (iii) a pharmaceutical excipient suitable for oral administration. In some embodiments, the pharmaceutical composition further contains: (iv) an effective amount of a third therapeutic agent.

In some embodiments, the pharmaceutical composition may be a pharmaceutical composition suitable for oral consumption. Pharmaceutical compositions containing a compound of the disclosure suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the compound of the disclosure into association with the carrier, which constitutes one or more necessary ingredients. In general, the pharmaceutical compositions are prepared by uniformly and intimately admixing the compound of the disclosure with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This disclosure further encompasses anhydrous pharmaceutical compositions and dosage forms comprising a compound described herein as the active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms containing a compound of the disclosure can be prepared using anhydrous or low moisture containing ingredients and low moisture or low humidity conditions. Pharmaceutical compositions and dosage forms containing a compound of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous pharmaceutical compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

In some embodiments, the pharmaceutical composition comprises a solid dispersion of a compound described herein, or a pharmaceutically acceptable salt thereof, in an inert carrier or a matrix generated by fusion, solvent evaporation, or melting-solvent processes known in the art. See Chiou W. L., Riegelman S., Pharmaceutical applications of solid dispersion systems, J. Pharm. Sci. 60(9):1281-1302 (1971).

The compound of the disclosure can be combined in an intimate admixture with a pharmaceutical carrier according to conventional pharmaceutical compounding techniques. The carrier can take a wide variety of forms depending on the form of preparation desired for administration. In preparing the pharmaceutical compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, colloidal silicon dioxide, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the pharmaceutical compositions of the disclosure to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 2 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical, and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof, carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof, polyoxyethylated vitamins and derivatives thereof, polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-1000 succinate, PEG-24 cholesterol, polyglyceryl-10-oleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the pharmaceutical composition may include a solubilizer to ensure good solubilization and/or dissolution of the compound of the disclosure and to minimize precipitation of the compound of the disclosure. This can be important for pharmaceutical compositions for non-oral use, e.g., pharmaceutical compositions for injection. A solubilizer may also be added to increase the solubility of the hydrophilic drug and/or other components, such as surfactants, or to maintain the pharmaceutical composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol (PEG), polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; polyethylene glycol 660 12-hydroxystearate; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, glycofurol, transcutol, propylene glycol, and dimethyl isosorbide. Particularly preferred solubilizers include sorbitol, glycerol, triacetin, ethyl alcohol, PEG having an average molecular weight of about 100 to about 8000 g/mole, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the pharmaceutical composition to a subject using conventional techniques, such as distillation or evaporation. Thus, if present, the solubilizer can be in a weight ratio of less than about 10%, less than about 25%, less than about 50%, about 100%, or up to less than about 200% by weight, based on the combined weight of the drug, and other excipients. If desired, very small amounts of solubilizer may also be used, such as less than about 5%, less than about 2%, less than about 1% or even less. Typically, the solubilizer may be present in an amount of less than about 1% to about 100%, more typically less than about 5% to less than about 25% by weight.

The pharmaceutical composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

### 6. Pharmaceutical Compositions for Injection

In some embodiments, the disclosure provides a pharmaceutical composition for injection containing a compound described herein, or a pharmaceutically acceptable salt thereof, and pharmaceutical excipients suitable for injection. Components and amounts of agents in the pharmaceutical compositions are as described herein.

The forms in which the pharmaceutical compositions of the disclosure may be incorporated for administration by injection include aqueous or oil suspensions or emulsions. Such pharmaceutical compositions may comprise sesame oil, corn oil, cottonseed oil, peanut oil, elixirs containing mannitol or dextrose, sterile water, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the disclosure, or a pharmaceutically acceptable salt thereof, in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### 7. Other Pharmaceutical Compositions

Pharmaceutical compositions may also be prepared from pharmaceutical compositions described herein and one or more pharmaceutically acceptable excipients suitable for topical, sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2004; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

### Exemplification

Compounds of the disclosure can be prepared by methods described in the General Schemes, procedures, and Examples set forth within, and by related methods known in the art. For example, Compounds of Formula I can be prepared by the general methods shown below. The general synthetic schemes are illustrative and are not meant to limit the possible techniques one skilled in the art may use to prepare the compounds described herein.

Starting materials and reagents used in these examples, when not prepared by a procedure described herein, are generally either commercially available, or are reported in the chemical literature, or may be prepared by using procedures described in the chemical literature.

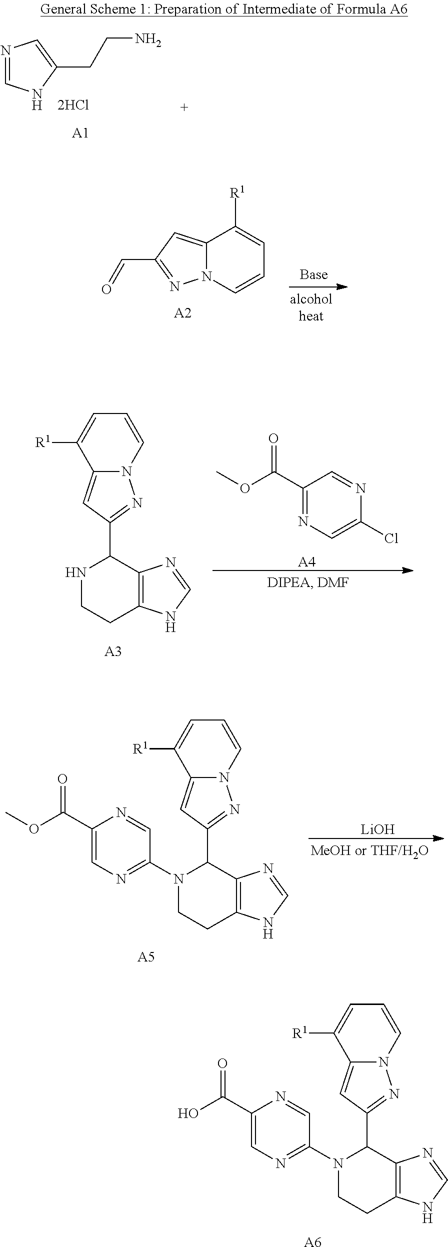

Intermediates of Formula A6 (wherein R¹ is hydrogen or fluoro) were obtained through the methods shown in General Scheme 1. Histamine (A1) and an aldehyde of formula A2 were reacted in a Pictet-Spengler reaction in the presence of a base such as sodium carbonate, an alcoholic solvent such as ethanol or methanol, and heat to afford the core amine of formula A3. To the extent that an imine by-product is formed during the Pictet-Spengler reaction, the imine by-product can be converted to the core amine of formula A3 by reaction with sodium borohydride in an alcoholic solvent (for example, methanol or ethanol). The core amine of formula A3 was then coupled to methyl 5-chloropyrazine-2-carboxylate (A4) using a nucleophilic substitution reaction in the presence of a base such as DIPEA or TEA and a solvent such as DMF, DMA, or NMP to form a compound of formula A5. The methyl ester of a compound of formula A5 was then hydrolyzed to the carboxylic acid (or lithium salt thereof) through reaction with LiOH in methanol/water or THF/water to afford compounds of formula A6.

Alternatively, an amine of A7,

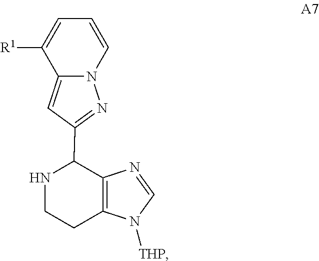

prepared in accordance with General Scheme 2 below, may also be used in the nucleophilic substitution reaction with methyl 5-chloropyrazine-2-carboxylate (A4), followed by deprotection of the tetrahydropyranyl (THP) protecting group at the desired stage under acidic conditions.

Racemic compounds of formula A3, formula A5, or formula A7 can each be separated into their R- and S-enantiomers (e.g., the stereogenic carbon atom of the imidazopyridinyl is in either the R-configuration or the S-configuration) through SFC separation, and then either one or both enantiomers of a compound of formula A3, formula A5, or formula A7 may be separately further reacted in General Scheme 3 and the other reactions described herein to afford enantiomerically enriched compounds.

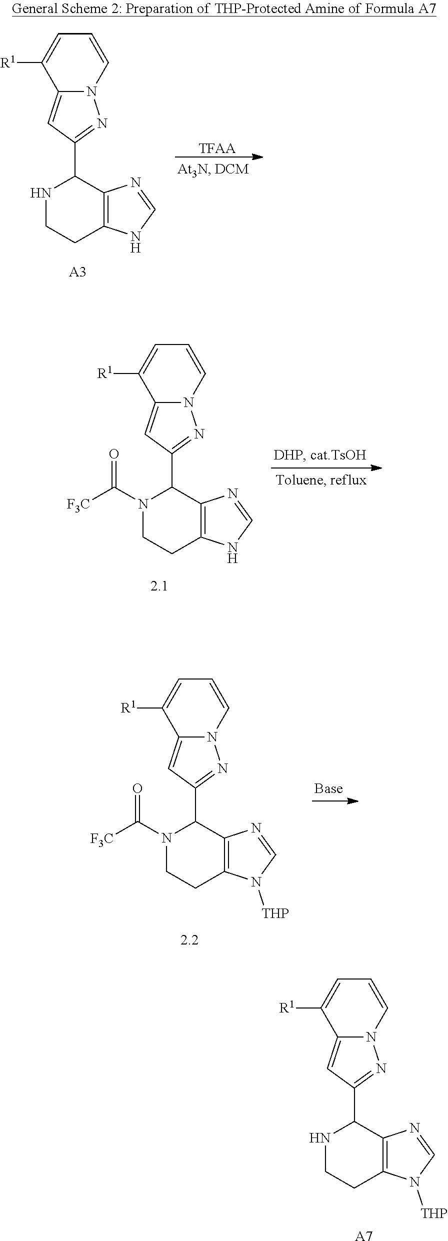

As shown in General Scheme 2, the amine of formula A3 may be protected with a trifluoroacetyl (TFA) protecting group through reaction with trifluoroacetic anhydride and a base such as triethyl amine to afford compounds of formula 2.1. One of the imidazole nitrogen atoms in the compound of formula 2.1 is protected with a THP protecting group via reaction with DHP and a catalytic amount of an acid such as TsOH to afford compounds of formula 2.2. The TFA protecting group in a compound of formula 2.2 may then be removed by reaction with a base such as 1M NaOH in THF or K₂CO₃ in MeOH to afford a compound of formula A7. The THP protecting group in compounds of formula A7 can be removed under acidic conditions such as 4M HCl in MeOH, TsOH in THF/water, or PPTS in ethanol.

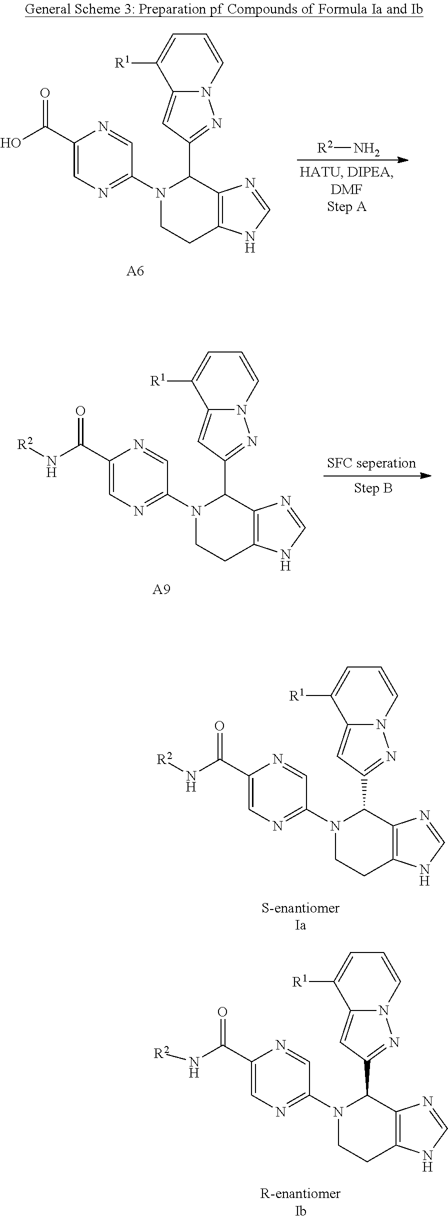

Enantiomers of Formula Ia and Ib were prepared in accordance with General Scheme 3. In Step A of General Scheme 3, the carboxylic acid moiety of a compound of formula A6 (or a lithium salt thereof) may be reacted with a R²-primary amine using an acid coupling reagent such as HATU along with a base such as DIPEA or TEA, at room temperature to afford racemic compounds of formula A9. Alternatively, as shown below in the preparation of Examples E and K, other acid coupling reagents known in the art, such as T4P, T3P, or EDCI and HOBt, may also be used in the coupling reaction along with a base, such as DIPEA or TEA. The racemic compounds of formula A9 may be separated into their S-enantiomer (compounds of Formula Ia) and R-enantiomer (compounds of Formula Ib) by SFC separation. Alternatively, enantiomerically enriched compounds of formula A6 can be used in Step A of General Scheme 3 to afford either a compound of Formula Ia or a compound of Formula Ib.

The present disclosure will be more fully understood by reference to the Examples described herein. The examples should not, however, be construed as limiting the scope of the present disclosure.

### Abbreviations and Terms List:

| | |
|---|---|
| aq | aqueous |
| min | minute(s) |
| hrs | hours |
| mL | milliliter |
| μL | microliter |
| mmol | millimole(s) |
| μmol | micromole(s) |
| mol | mole(s) |
| M | molar |
| eq | equivalents |
| LCMS | Liquid chromatography mass spectrometry |
| NMR | nuclear magnetic resonance |
| TLC | thin layer chromatography |
| HPLC | high-performance liquid chromatography |
| SFC | supercritical fluid chromatography |
| sat | saturated |
| ° C. | degrees Celsius |
| rt | room temperature |
| N₂ | nitrogen gas |
| Hz | Hertz |
| δ | chemical shift |
| s | singlet |
| d | doublet |
| t | triplet |
| q | quartet |
| m | multiplet |
| br | broad |
| dd | doublet of doublets |
| ddd | doublet of doublet or doublets |
| td | triplet of doublets |
| dt | doublet of triplets |
| CDCl₃ | chloroform-d |
| CD₃OD | methanol-d₄ |
| DMSO-d₆ | dimethyl sulfoxide-d₆ |

### Solvents and Reagents:

| | |
|---|---|
| DCM | dichloromethane |
| DMAc or | dimethylacetamide |
| DMA | |
| DMF | dimethylformamide |
| DMSO | dimethyl sulfoxide |
| EtOH | ethyl alcohol |
| EtOAc | ethyl acetate |
| i-PrOH or | isopropanol |
| IPA | |
| MeOH | methanol |
| TAA | tert-amyl alcohol or 2-methylbutan-2-ol |
| MeCN or | acetonitrile |
| ACN | |
| PE | petroleum ether |
| H₂O | water |
| THF | tetrahydrofuran |
| NMP | N-methyl-2-pyrrolidone |
| HCl | hydrochloric acid |
| TFA | trifluoroacetic acid |
| TFAA | trifluoroacetic anhydride |
| TosOH or | p-toluenesulfonic acid |
| TsOH | |
| PPTS | pyridinium p-toluenesulfonate |
| NH₄C1 | ammonium chloride |
| LiOH | lithium hydroxide |
| NaOH | Sodium hydroxide |
| K₂CO₃ | potassium carbonate |
| Na₂CO₃ | sodium carbonate |
| Na₂SO₄ | sodium sulfate |
| NaHCO₃ | sodium bicarbonate |
| Et₃N or TEA | triethylamine |
| DIPEA or | N,N-diisopropylethylamine |
| DIEA | |
| DHP | dihyropyran |
| NH₃ | ammonia |
| DIBAL-H | diisobutylaluminium hydride |
| NaOMe | sodium methoxide |
| IBX | 2-iodoxybenzoic acid |
| EDCI | 1-ethyl-3-(3-dimethylaminopropyl)carbodiimide |
| HOBt | 1-hydroxybenzotriazole |
| HATU | 1-[Bis(dimethylamino)methylene]-1H-1,2,3-triazolo[4,5- |
| | b]pyridinium 3-oxid hexafluorophosphate |
| T3P | Propanephosphonic acid anhydride |
| T4P | 3-[2,6,8-trioxo-9-[(2R,3R,4R)-2,3,4,5- |
| | tetrahydroxypentyl]-3H-purin-7-yl]propyl dihydrogen |
| | phosphate |
| Pd/C | palladium on carbon |
| Pd(PPh₃)₂Cl₂ | bis(triphenylphosphine)palladium(II) dichloride |
| CuI | copper iodide |

### Preparation of Intermediate Pyrazolo[1,5-a]pyridine-2-carbaldehyde (B3)

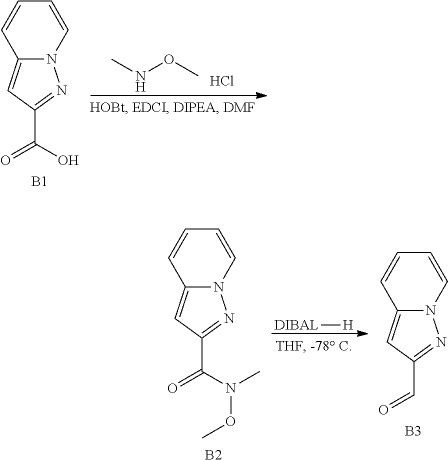

### Step 1: Preparation of N-methoxy-N-methyl-pyrazolo[1,5-a]pyridine-2-carboxamide (B2)

To a solution of pyrazolo[1,5-a]pyridine-2-carboxylic acid (B1) (12.5 g, 77.1 mmol) and N-methoxymethanamine (9.42 g, 96.6 mmol, 1.25 eq, HCl) in DMF (200 mL) was added EDCI (22.17 g, 116 mmol, 1.5 eq), DIPEA (29.89 g, 231 mmol, 40.3 mL, 3 eq) and HOBt (15.63 g, 116 mmol, 1.5 eq). The reaction mixture was stirred at rt for 12 hrs. Reaction progress was checked using TLC (PE:EtOAc=1:1). The reaction mixture was combined with the reaction mixture from another reaction performed using 12.5 g of B1 and concentrated to dryness. The residue was dissolved in DCM (200 mL), and the organic layer washed with sat. aq. Na₂CO₃ solution (100 mL) and brine (100 mL), dried over Na₂SO₄, filtered, and concentrated to dryness. The residue was purified by column chromatography on silica gel (PE:EtOAc=20:1 to 1:1) to give B2 (30.5 g, 96% yield). ¹H NMR (400 MHz, CDCl₃) δ 8.47-8.54 (m, 1H), 7.59 (d, 1H), 7.16 (ddd, 1H), 7.02 (s, 1H), 6.86 (td, 1H), 3.81 (s, 3H), 3.51 (s, 3H).

### Step 2: Preparation of pyrazolo[1,5-a]pyridine-2-carbaldehyde (B3)

To a solution of N-methoxy-N-methyl-pyrazolo[1,5-a]pyridine-2-carboxamide (B2) (15 g, 73.1 mmol) in THF (150 mL) was added DIBAL-H (1 M, 146.2 mL, 2 eq) dropwise at −78° C. under N₂, and then the reaction mixture was stirred at −78° C. for 2 hrs under N₂ atmosphere. Reaction progress was tracked using TLC (PE:EtOAc=1:1). The reaction mixture was combined with the reaction mixture from another reaction performed using 15 g of B2 and was quenched by the addition of sat. aq. NH₄Cl solution (200 mL) slowly and stirred for 15 min. 1 M HCl solution was added until a clear solution was observed. The aqueous portion was extracted with EtOAc (250 mL×3), and the combined organic layer was washed with water (100 mL) and brine (100 mL), dried over Na₂SO₄, filtered, and concentrated to dryness. The residue was purified by column chromatography on silica gel (PE:EtOAc=20:1 to 1:1) to give B3 (14.2 g). ¹H NMR (400 MHz, DMSO-d₆) δ 10.14 (s, 1H), 8.81 (dd, 1H), 7.84 (d, 1H), 7.34 (t, 1H), 7.10-7.15 (m, 2H).

### Preparation of Intermediate 4-(pyrazolo[1,5-a]pyridin-2-yl)-4,5,6,7-tetrahydro-1H-imidazo[4,5-c]pyridine (B4) According to General Scheme 1

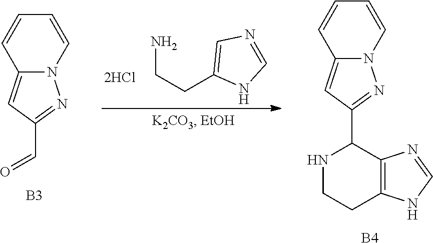

A mixture of pyrazolo[1,5-a]pyridine-2-carbaldehyde (B3) (9.2 g, 63.0 mmol), 2-(1H-imidazol-5-yl)ethanamine (15.06 g, 81.8 mmol, 2HCl salt) and K₂CO₃ (17.40 g, 126 mmol) in EtOH (300 mL) was stirred at 80° C. for 16 hrs under N₂. Reaction progress was checked using LCMS. The reaction mixture was combined with another reaction mixture performed using 5 g of B3 and filtered, and the filtrate was concentrated to dryness. The residue was purified by column chromatography on silica gel (DCM:MeOH=20:1 to 10:1) to give B4 (11.4 g). ¹H NMR (400 MHz, CD₃OD) δ 8.48 (d, 1H), 7.54-7.62 (m, 2H), 7.09-7.29 (m, 1H), 6.82-6.90 (m, 1H), 6.44 (s, 1H), 5.28 (s, 1H), 3.28 (dt, 1H), 3.04-3.12 (m, 1H), 2.73-2.80 (m, 2H); LC-MS: m/z 240.2 (M+H)⁺.

### Preparation of Intermediate 4-fluoropyrazolo[1,5-a]pyridine-2-carbaldehyde (B10)

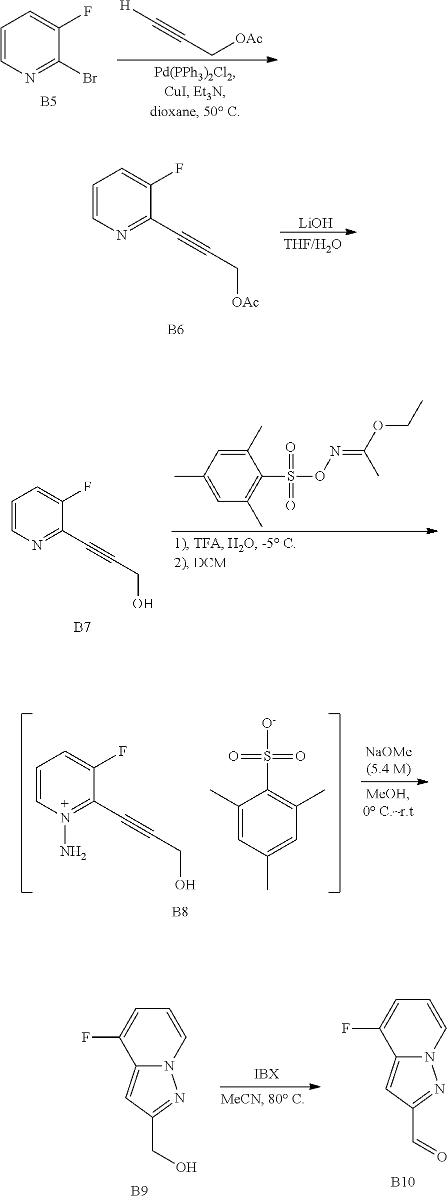

### Step 1: Preparation of 3-(3-fluoro-2-pyridyl)prop-2-yn-1yl acetate (B6)

A mixture of 2-bromo-3-fluoro-pyridine (B5) (30 g, 170 mmol), prop-2-yn-1yl acetate (23.41 g, 239 mmol, 1.4 eq), Pd(PPh₃)₂Cl₂ (5.98 g, 8.52 mmol, 0.05 eq), CuI (1.62 g, 8.52 mmol, 0.05 eq) and TEA (51.75 g, 511 mmol, 71.2 mL, 3 eq) in dioxane (300 mL) was degassed and purged with N₂ gas 3 times, and then the reaction mixture was stirred at 50° C. for 6 hrs under N₂ atmosphere. Reaction progress was checked using TLC (PE:EtOAc=5:1). The reaction mixture was concentrated to dryness. EtOAc (600 mL) was added to the residue, and the organic portion washed with water (300 mL) and brine (300 mL), dried over Na₂SO₄, filtered, and concentrated to dryness. The residue was purified by flash silica gel chromatography (ISCO®; 120 g SepaFlash® Silica Flash Column, eluent of 0~20% EtOAc/PE, gradient @120 mL/min) to give B6 (23.4 g, 71% yield). ¹H NMR (400 MHz, CDCl₃) δ 8.43 (d, 1H), 7.46 (td, 1H), 7.32 (dt, 1H), 4.99 (s, 2H), 2.16 (s, 3H).

### Step 2: Preparation of 3-(3-fluoro-2-pyridyl)prop-2-yn-1-ol (B7)

To a solution of 3-(3-fluoro-2-pyridyl)prop-2-ynyl acetate (B6) (23.4 g, 121 mmol) in THF (300 mL) and H₂O (150 mL) and was added LiOH·H₂O (5.34 g, 127 mmol, 1.05 eq) at 0° C. The reaction mixture was stirred at rt for 2 hrs. Reaction progress was checked using TLC (PE:EtOAc=1:1). EtOAc (600 mL) was added to the reaction mixture, and the organic layer washed with H₂O (400 mL) and brine (400 mL), dried over Na₂SO₄, filtered and concentrated to dryness to afford B7 (17.5 g), which was used without further purification. ¹H NMR (400 MHz, DMSO-d₆) δ 8.34-8.53 (m, 1H), 7.72-7.88 (m, 1H), 7.50 (dt, 1H), 5.54 (t, 1H), 4.38 (d, 2H).

### Step 3: Preparation of 1-amino-3-fluoro-2-(3-hydroxyprop-1-yn-1-yl)pyridin-1-ium 2,4,6-trimethylbenzenesulfonate (B8)

To a mixture of H₂O (35 mL, 1.94 mol, 17.3 eq) and TFA (308.0 g, 2.70 mol, 200 mL, 24.0 eq) was added ethyl (1E)-N-(2,4,6-trimethylphenyl)sulfonyloxyethanimidate (33.70 g, 118 mmol, 1.05 eq) at 0° C., and the reaction mixture was stirred at 0° C. for 2 hrs. The reaction was quenched with ice-water (40 mL). The precipitate was filtered and washed with water (20 mL×2). The precipitate was then dissolved in DCM (200 mL) and dried over Na₂SO₄ and filtered. To the filtrate was added 3-(3-fluoro-2-pyridyl)prop-2-yn-1-ol (B7) (17 g, 112 mmol, 1 eq) at 0° C. The reaction mixture was warmed to rt for 16 hrs. Reaction progress was checked using TLC (PE:EtOAc=1:1). TBME (400 mL) was slowly added to the reaction mixture, and the precipitate was collected by filtration. The precipitate was rinsed with TBME (200 mL×2) to afford B8 (26 g), which was used without further purification. ¹H NMR (400 MHz, DMSO-d₆) δ 8.66-8.88 (m, 3H), 8.32 (t, 1H), 8.01 (dt, 1H), 6.75 (s, 2H), 4.58 (s, 2H), 3.17 (s, 1H), 2.49 (s, 6H), 2.17 (s, 3H).

### Step 4: Preparation of (4-fluoropyrazolo[1,5-a]pyridin-2-yl) methanol (B9)

To a solution of 1-amino-3-fluoro-2-(3-hydroxyprop-1-yn-1-yl)pyridin-1-ium 2,4,6-trimethylbenzenesulfonate (B8) (26 g, 71.0 mmol) in MeOH (250 mL) was added NaOMe (5.4 M, 26.3 mL, 2 eq) at 0° C. The reaction mixture was stirred at rt for 1 hr. The reaction progress was tracked by TLC (PE:EtOAc=1:1). The reaction mixture was quenched by the addition of ice-cold H₂O (200 mL), concentrated to remove most of the MeOH, and extracted with EtOAc (200 mL×3). The combined organic layer was washed with brine (400 mL), dried over Na₂SO₄, filtered, and concentrated to dryness to give B9 (7.5 g), which was used without further purification. ¹H NMR (400 MHz, CDCl₃) δ 8.27 (d, 1H), 6.62-6.88 (m, 3H), 4.94 (s, 2H).

### Step 5: Preparation of 4-fluoropyrazolo[1,5-a]pyridine-2-carbaldehyde (B10)

To a solution of (4-fluoropyrazolo[1,5-a]pyridin-2-yl)methanol (B9) (7.5 g, 45.1 mmol) in MeCN (150 mL) was added IBX (15.17 g, 54.2 mmol, 1.2 eq). The reaction mixture was stirred at 80° C. for 3 hrs. Reaction progress was checked using TLC (PE:EtOAc=5:1). The reaction mixture was filtered and concentrated to dryness. The residue was purified by flash silica gel chromatography (ISCO®; 40 g SepaFlash® Silica Flash Column, Eluent of 0~4% EtOAc/PE, gradient @40 mL/min) to give B10 (4 g, 54% yield). ¹H NMR (400 MHz, CDCl₃) δ 10.22 (s, 1H), 8.33-8.41 (m, 1H), 7.20 (d, 1H), 6.85-6.98 (m, 2H).

### Preparation of Intermediate 4-pyrazolo[1,5-a]pyridin-2-yl-1-tetrahydropyran-2-yl-4,5,6,7-tetrahydroimidazo[4,5-c]pyridine According to General Scheme 2

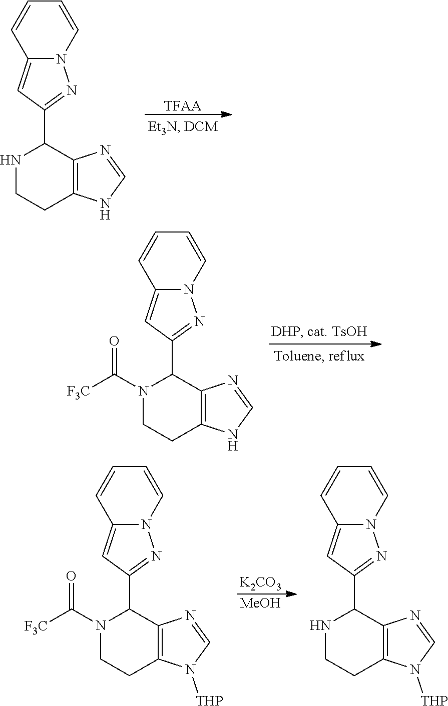

### Step 1: Preparation of 2,2,2-trifluoro-1-(4-pyrazolo[1,5-a]pyridin-2-yl-1,4,6,7-tetrahydroimidazo[4,5-c]pyridin-5-yl)ethenone

To a solution of 4-pyrazolo[1,5-a]pyridin-2-yl-4,5,6,7-tetrahydro-1H-imidazo[4,5-c]pyridine (1.2 g, 5.02 mmol) in DCM (25 mL), Et₃N (2.03 g, 20.1 mmol, 2.79 mL) and TFAA (2.74 g, 13.0 mmol, 1.81 mL) were added dropwise at 0° C. After addition was completed, the reaction mixture was stirred at rt for 15 hrs. DCM (50 mL) was added to the reaction mixture, and the organic portion washed with water (30 mL) and brine (30 mL), dried over Na₂SO₄, filtered, and concentrated to dryness. The residue was purified by flash silica gel chromatography (ISCO®; 4 g SepaFlash® Silica Flash Column, Eluent 0~10% DCM/MeOH @20 mL/min) to give 2,2,2-trifluoro-1-(4-pyrazolo[1,5-a]pyridin-2-yl-1,4,6,7-tetrahydroimidazo[4,5-c]pyridin-5-yl)ethanone (1.23 g, 69.6% yield). ¹H NMR (400 MHz, DMSO-d₆) δ 8.61-8.66 (m, 1H), 7.61-7.66 (m, 2H), 7.18-7.23 (m, 1H), 6.87 (td, 1H), 6.60 (s, 1H), 6.51-6.54 (m, 1H), 4.09 (br d, 1H), 3.67-3.77 (m, 0.2H), 3.38-3.45 (m, 0.8H), 2.69-2.85 (m, 2H); LCMS: m/z 336.1 [M+H]⁺.

### Step 2: Preparation of 2,2,2-trifluoro-1-(4-pyrazolo[1,5-a]pyridin-2-yl-1-tetrahydropyran-2-yl-6,7-dihydro-4H-imidazo[4,5-c]pyridin-5-yl)ethenone

To a solution of 2,2,2-trifluoro-1-(4-pyrazolo[1,5-a]pyridin-2-yl-1,4,6,7-tetrahydroimidazo[4,5-c]pyridin-5-yl)ethanone (1.23 g, 3.67 mmol) in toluene (16 mL) were added TsOH (63.2 mg, 367 mol) and 3,4-dihydro-2H-pyran (926 mg, 11.0 mmol, 1.01 mL). The reaction mixture was stirred at 100° C. for 15 hrs. The reaction mixture was cooled to rt, and DCM was added (200 mL). The organic portion was washed with sat. aq. Na₂CO₃ solution (60 mL) and brine (50 mL), dried over Na₂SO₄, filtered, and concentrated to dryness. The residue was purified by column chromatography (SiO₂, DCM:MeOH=100:1 to 10:1) to give 2,2,2-trifluoro-1-(4-pyrazolo[1,5-a]pyridin-2-yl-1-tetrahydropyran-2-yl-6,7-dihydro-4H-imidazo[4,5-c]pyridin-5-yl)ethanone (1.1 g, 70.5% yield). ¹H NMR (400 MHz, DMSO-d₆) δ 8.58-8.66 (m, 1H), 7.80 (d, 1H), 7.63 (d, 1H), 7.20 (dd, 1H), 6.83-6.89 (m, 1H), 6.48-6.57 (m, 2H), 5.18-5.32 (m, 1H), 4.05-4.16 (m, 1H), 3.68-3.79 (m, 1H), 3.56-3.66 (m, 1H), 3.37-3.47 (m, 1H), 2.71-2.90 (m, 2H), 1.95 (br d, 2H), 1.42-1.54 (m, 4H); LCMS: m/z 420.2 [M+H]⁺.

### Step 3: Preparation of 4-pyrazolo[1,5-a]pyridin-2-yl-1-tetrahydropyran-2-yl-4,5,6,7-tetrahydroimidazo[4,5-c]pyridine

To a solution of 2,2,2-trifluoro-1-(4-pyrazolo[1,5-a]pyridin-2-yl-1-tetrahydropyran-2-yl-6,7-dihydro-4H-imidazo[4,5-c]pyridin-5-yl)ethanone (1.1 g, 2.62 mmol) in MeOH (10 mL) was added K₂CO₃ (1.63 g, 11.8 mmol). The reaction mixture was stirred at 60° C. for 14 hrs. The reaction mixture was cooled to rt and filtered, and the filtrate was concentrated to dryness. The residue was purified by column chromatography (SiO₂, DCM:MeOH=100:1 to 10:1) to give 4-pyrazolo[1,5-a]pyridin-2-yl-1-tetrahydropyran-2-yl-4,5,6,7-tetrahydroimidazo[4,5-c]pyridine (580 mg, 67.8% yield). ¹H NMR (400 MHz, DMSO-d₆) δ 8.57 (br d, 1H), 7.54-7.63 (m, 2H), 7.13 (dd, 1H), 6.80 (br t, 1H), 6.33 (d, 1H), 5.14-5.24 (m, 1H), 5.03 (br s, 1H), 3.95 (br d, 1H), 3.56-3.67 (m, 1H), 3.05-3.14 (m, 1H), 2.84-3.00 (m, 2H), 1.87-2.03 (m, 3H), 1.59-1.80 (m, 2H), 1.54 (br d, 2H); LCMS: m/z 324.2 [M+H]⁺.

### Preparation of Intermediate 5-(4-pyrazolo[1,5-a]pyridin-2-yl-1,4,6,7-tetrahydroimidazo[4,5-c]pyridin-5-yl)pyrazine-2-carboxylic acid (B12) According to General Scheme 1

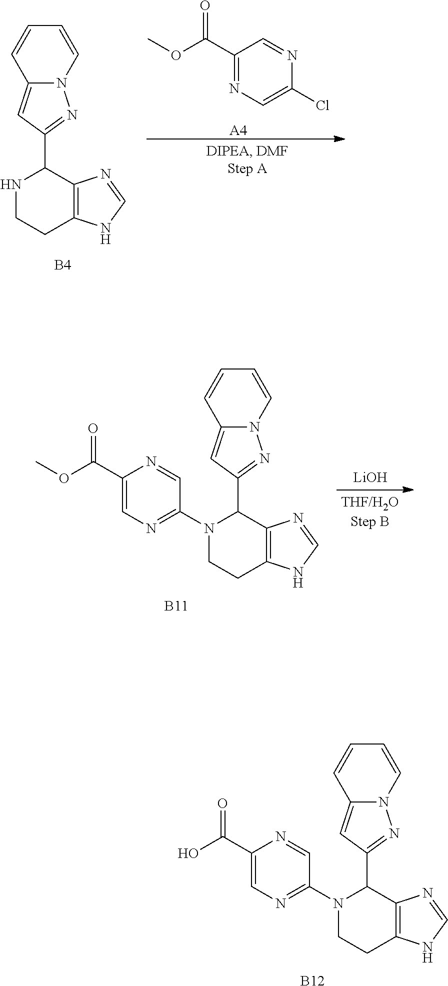

### Step A: Synthesis of methyl 5-(4-pyrazolo[1,5-a]pyridin-2-yl-1,4,6,7-tetrahydroimidazo[4,5-c]pyridin-5-yl)pyrazine-2-carboxylate (B11)

To a solution of 4-pyrazolo[1,5-a]pyridin-2-yl-4,5,6,7-tetrahydro-1H-imidazo[4,5-c]pyridine (B4) (3 g, 12.5 mmol, 1 eq) and methyl 5-chloropyrazine-2-carboxylate (A4) (3.03 g, 17.6 mmol, 1.4 eq) in DMF (20 mL) was added DIPEA (6.48 g, 50.2 mmol, 8.74 mL, 4 eq). The reaction mixture was stirred at 20° C. for 12 hrs. DCM (50 mL) was added to the reaction mixture. The organic layer was washed with sat. NaHCO₃ solution (30 mL) and brine (30 mL), dried over Na₂SO₄, filtered, and concentrated to dryness. The residue was purified by flash silica gel chromatography (ISCO®; 40 g SepaFlash® Silica Flash Column, Eluent of 0-10% MeOH:DCM @40 mL/min) to give B11 (2.86 g, 59.8% yield). LCMS (Shimadzu LC-20AD & MS 2020): m/z 376.2 [M+H]⁺.

### Step B: Synthesis of 5-(4-pyrazolo[1,5-a]pyridin-2-yl-1,4,6,7-tetrahydroimidazo[4,5-c]pyridin-5-yl)pyrazine-2-carboxylic acid (B12)

To a solution of methyl 5-(4-pyrazolo[1,5-a]pyridin-2-yl-1,4,6,7-tetrahydroimidazo[4,5-c]pyridin-5-yl)pyrazine-2-carboxylate (B11) (2.86 g, 7.62 mmol, 1 eq) in THF (30 mL) was added LiOH·H₂O (480 mg, 11.4 mmol, 1.5 eq) in H₂O (10 mL), and the reaction mixture was stirred at 25° C. for 2 hrs. The reaction mixture was concentrated in vacuum, and the aqueous phase was adjusted to a pH of approximately 6 by the addition of 4M HCl solution. The precipitate was filtered and dried in vacuum to give B12 (3 g). ¹H NMR (Bruker AV-400, 400 MHz, DMSO-d₆) δ 11.99-12.46 (m, 1H), 8.67 (d, 1H), 8.56-8.63 (m, 2H), 7.55-7.65 (m, 2H), 7.12-7.23 (m, 1H), 6.82 (td, 2H), 6.56 (s, 1H), 4.87 (br dd, 1H), 3.51-3.59 (m, 1H), 2.80-2.91 (m, 1H), 2.69-2.75 (m, 1H); LCMS: m/z 362.1 [M+H]⁺.

### Preparation of Intermediate 5-[4-(4-fluoropyrazolo[1,5-a]pyridin-2-yl)-1,4,6,7-tetrahydroimidazo[4,5-c]pyridin-5-yl]pyrazine-2-carboxylic acid (B15) According to General Scheme 1

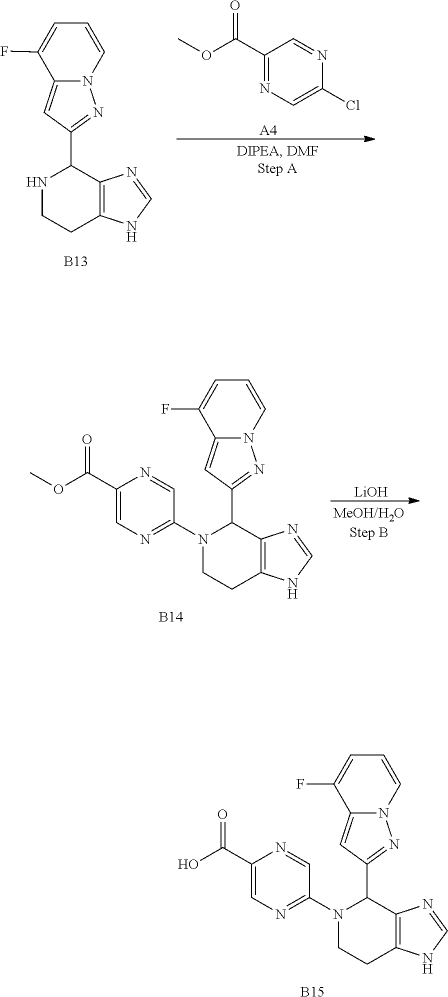

### Step A: Preparation of methyl 5-[4-(4-fluoropyrazolo[1,5-a]pyridin-2-yl)-1,4,6,7-tetrahydroimidazo[4,5-c]pyridin-5-yl]pyrazine-2-carboxylate (B14)

To a solution of 4-(4-fluoropyrazolo[1,5-a]pyridin-2-yl)-4,5,6,7-tetrahydro-1H-imidazo[4,5-c]pyridine (B13) (2 g, 7.77 mmol, 1 eq) in DMF (20 mL) was added DIPEA (3.01 g, 23.3 mmol, 4.06 mL, 3 eq) and methyl 5-chloropyrazine-2-carboxylate (A4) (1.48 g, 8.55 mmol, 1.1 eq). The reaction mixture was stirred at 60° C. for 14 hrs and then cooled to rt. Reaction progress was checked using LCMS (Shimadzu LC-20AD &MS 2020). Sat. aq. NaHCO₃ solution (20 mL) was added to the reaction mixture, and the aqueous portion was extracted with EtOAc (3×60 mL). The combined organic layer was washed with brine (20 mL), dried over Na₂SO₄, filtered, and concentrated in vacuum to dryness. The residue was purified by flash silica gel chromatography (ISCO®; 40 g SepaFlash® Silica Flash Column, Eluent of 0~4.2% MeOH:DCM @40 mL/min) to give B14 (1.7 g, 41.7% yield). ¹H NMR (Bruker AV-400, 400 MHz, METHANOL-d₄) δ 8.79 (d, 1H), 8.52 (d, 1H), 8.31 (d, 1H), 7.65 (s, 1H), 6.90-6.96 (m, 2H), 6.79 (td, 1H), 6.67 (s, 1H), 4.86-4.91 (m, 1H), 3.91 (s, 3H), 3.59-3.70 (m, 1H), 2.90-3.04 (m, 1H), 2.80 (dd, 1H); LCMS: m/z 394.2 [M+H]⁺.

### Step B: Preparation of 5-[4-(4-fluoropyrazolo[1,5-a]pyridin-2-yl)-1,4,6,7-tetrahydroimidazo[4,5-c]pyridin-5-yl]pyrazine-2-carboxylic acid (B15)

To a solution of methyl 5-[4-(4-fluoropyrazolo[1,5-a]pyridin-2-yl)-1,4,6,7-tetrahydroimidazo[4,5-c]pyridin-5-yl]pyrazine-2-carboxylate (B14) (1.7 g, 4.32 mmol, 1 eq) in MeOH (10 mL) and H₂O (2 mL) was added LiOH·H₂O (199 mg, 4.75 mmol, 1.1 eq). The reaction mixture was stirred at 15° C. for 14 hrs. Reaction progress was checked using LCMS (Shimadzu LC-20AD &MS 2020). The reaction mixture was concentrated under reduced pressure. HCl (2 M) was added to the residue until the pH of the reaction mixture was approximately pH 4. The precipitate was collected by filtration and dried under reduced pressure to give B15 (1.5 g, 91.5% yield). ¹H NMR (Bruker AV-400, 400 MHz, DMSO-d₆) δ 11.47-13.13 (m, 2H), 8.68 (d, 1H), 8.61 (s, 1H), 8.54 (d, 1H), 7.58 (s, 1H), 7.10 (dd, 1H), 6.78-6.89 (m, 2H), 6.73 (s, 1H), 4.88 (br d, 1H), 3.51-3.61 (m, 1H), 2.79-2.96 (m, 1H), 2.65-2.78 (m, 1H); LCMS: m/z 380.2 [M+H]⁺.

### Preparation of Intermediates methyl (S)-5-(4-(4-fluoropyrazolo[1,5-a]pyridin-2-yl)-1,4,6,7-tetrahydro-5H-imidazo[4,5-c]pyridin-5-yl)pyrazine-2-carboxylate (B14-S) and methyl (R)-5-(4-(4-fluoropyrazolo[1,5-a]pyridin-2-yl)-1,4,6,7-tetrahydro-5H-imidazo[4,5-c]pyridin-5-yl)pyrazine-2-carboxylate (B14-R)

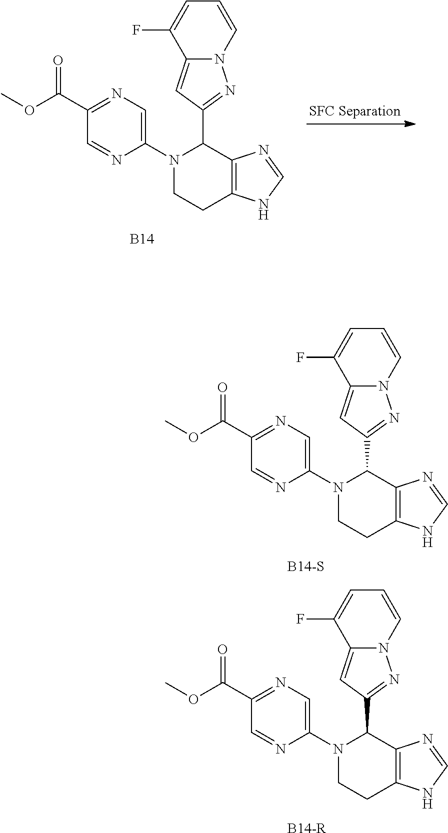

SFC Separation Procedure: Methyl 5-(4-(4-fluoropyrazolo[1,5-a]pyridin-2-yl)-1,4,6,7-tetrahydro-5H-imidazo[4,5-c]pyridin-5-yl)pyrazine-2-carboxylate (B14) (6.5 g) was separated by SFC (column: CHIRALPAK IH (150×20 mm, 5 μm); mobile phase: [CO₂-MeOH(0.10% NH₃H₂O)]; B %: 30%, isocratic elution mode) to afford Enantiomer #1 (3 g, Rt=2.8 min) and Enantiomer #2 (2.8 g, Rt=4.8 min).

Enantiomer #1, methyl (R)-5-(4-(4-fluoropyrazolo[1,5-a]pyridin-2-yl)-1,4,6,7-tetrahydro-5H-imidazo[4,5-c]pyridin-5-yl)pyrazine-2-carboxylate (B14-R): ¹H NMR (400 MHz, DMSO-d₆) δ 12.04 (s, 1H), 8.70 (s, 1H), 8.64 (s, 1H), 8.53 (d, 1H), 7.59 (s, 1H), 7.20-7.01 (m, 1H), 6.93-6.77 (m, 2H), 6.74 (s, 1H), 5.05-4.64 (m, 1H), 3.82 (s, 3H), 3.66-3.51 (m, 1H), 2.97-2.83 (m, 1H), 2.82-2.70 (m, 1H); LC-MS: m/z 394.1 [M+H]⁺; 100% ee.

Enantiomer #2, methyl (S)-5-(4-(4-fluoropyrazolo[1,5-a]pyridin-2-yl)-1,4,6,7-tetrahydro-5H-imidazo[4,5-c]pyridin-5-yl)pyrazine-2-carboxylate (B14-S): ¹H NMR (400 MHz, DMSO-d₆) δ 12.04 (s, 1H), 8.70 (s, 1H), 8.64 (s, 1H), 8.53 (d, 1H), 7.59 (s, 1H), 7.20-7.01 (m, 1H), 6.93-6.77 (m, 2H), 6.74 (s, 1H), 5.05-4.64 (m, 1H), 3.82 (s, 3H), 3.66-3.51 (m, 1H), 2.97-2.83 (m, 1H), 2.82-2.70 (m, 1H); LC-MS: m/z 394.1 [M+H]⁺; 100% ee.

### Preparation of N-cyclopropyl-5-[(4S)-4-(4-fluoropyrazolo[1,5-a]pyridin-2-yl)-1,4,6,7-tetrahydroimidazo[4,5-c]pyridin-5-yl]pyrazine-2-carboxamide (Example A)

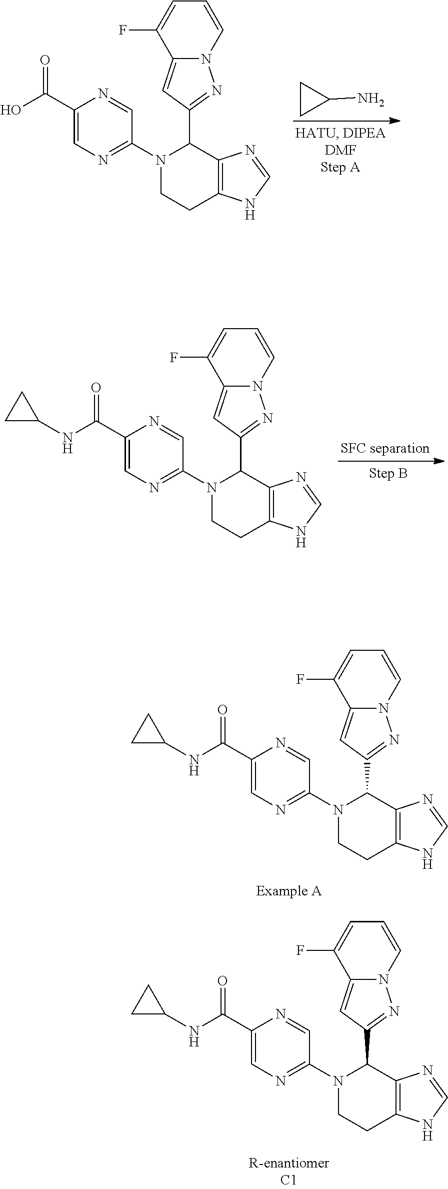

### Step A: Preparation of N-cyclopropyl-5-[4-(4-fluoropyrazolo[1,5-a]pyridin-2-yl)-1,4,6,7-tetrahydroimidazo[4,5-c]pyridin-5-yl]pyrazine-2-carboxamide

To a solution of 5-[4-(4-fluoropyrazolo[1,5-a]pyridin-2-yl)-1,4,6,7-tetrahydroimidazo[4,5-c]pyridin-5-yl]pyrazine-2-carboxylic acid (1.5 g, 3.95 mmol, 1 eq) and cyclopropanamine (339 mg, 5.93 mmol, 411 L, 1.5 eq) in DMF (10 mL) was added HATU (1.80 g, 4.74 mmol, 1.2 eq) and DIPEA (1.53 g, 11.9 mmol, 2.07 mL, 3 eq). The reaction mixture was stirred at 15° C. for 14 hrs. Reaction progress was checked using LCMS (Shimadzu LC-20AD &MS 2020). Sat. aq. NaHCO₃ solution (25 mL) was added to the reaction mixture, and the aqueous portion extracted with EtOAc (3×60 mL). The combined organic layer was washed with brine (20 mL), dried over Na₂SO₄, filtered and concentrated to dryness. The residue was purified by flash silica gel chromatography (ISCO®; 40 g SepaFlash® Silica Flash Column, Eluent of 0~5% MeOH:DCM @40 mL/min) to give N-cyclopropyl-5-[4-(4-fluoropyrazolo[1,5-a]pyridin-2-yl)-1,4,6,7-tetrahydroimidazo[4,5-c]pyridin-5-yl]pyrazine-2-carboxamide (960 mg, 56.9% yield). ¹H NMR (Bruker AV-400, 400 MHz, DMSO-d₆) δ 11.93-12.35 (m, 1H), 8.59-8.69 (m, 1H), 8.44-8.57 (m, 2H), 8.31 (br d, 1H), 7.56 (s, 1H), 6.97-7.19 (m, 1H), 6.78-6.91 (m, 1H), 6.72 (br d, 2H), 4.92 (br dd, 1H), 3.45-3.62 (m, 1H), 2.64-2.91 (m, 3H), 0.52-0.75 (m, 4H); ¹⁹F NMR (Bruker AV-400, 376 MHz, DMSO-d₆) δ −125.2; LCMS: m/z 419.2 [M+H]⁺.

### Step B: SFC Separation

N-cyclopropyl-5-[4-(4-fluoropyrazolo[1,5-a]pyridin-2-yl)-1,4,6,7-tetrahydroimidazo[4,5-c]pyridin-5-yl]pyrazine-2-carboxamide (900 mg, 2.15 mmol) was separated by SFC (Waters SFC Prep 150 AP System; column: DAICEL CHIRALPAK AY-H(250 mm*30 mm, 10 μm); mobile phase: [CO₂-EtOH(0.1% NH₃H₂O)]; B %: 50%, isocratic elution mode) to give Enantiomer #1 (200 mg, Rt=2.137 min) and Enantiomer #2 (180 mg, Rt=4.225 min).

Enantiomer #1 (C1): N-cyclopropyl-5-[(4R)-4-(4-fluoropyrazolo[1,5-a]pyridin-2-yl)-1,4,6,7-tetrahydroimidazo[4,5-c]pyridin-5-yl]pyrazine-2-carboxamide, ¹H NMR (Bruker AV-400; 400 MHz, METHANOL-d₄) δ 8.73 (d, 1H), 8.45 (d, 1H), 8.32 (d, 1H), 7.65 (s, 1H), 6.75-7.04 (m, 3H), 6.66 (s, 1H), 4.83-4.83 (m, 1H), 3.57-3.71 (m, 1H), 2.91-3.03 (m, 1H), 2.73-2.88 (m, 2H), 0.76-0.97 (m, 2H), 0.50-0.70 (m, 2H); ¹⁹F NMR (Bruker AV-400, 376 MHz, METHANOL-d₄) 6-126.7; LCMS (Agilent Prime-6125B): m/z 419.2 [M+H]⁺; SFC (Waters UPCC with PDA Detector): 100% ee.

Enantiomer #2 (Example A): N-cyclopropyl-5-[(4S)-4-(4-fluoropyrazolo[1,5-a]pyridin-2-yl)-1,4,6,7-tetrahydroimidazo[4,5-c]pyridin-5-yl]pyrazine-2-carboxamide, ¹H NMR (Bruker AV-400, 400 MHz, METHANOL-d₄) δ 8.73 (d, 1H), 8.45 (d, 1H), 8.32 (d, 1H), 7.64 (s, 1H), 6.76-6.98 (m, 3H), 6.66 (s, 1H), 4.83-4.83 (m, 1H), 3.57-3.71 (m, 1H), 2.91-3.03 (m, 1H), 2.74-2.87 (m, 2H), 0.77-0.88 (m, 2H), 0.59-0.71 (m, 2H); ¹⁹F NMR (Bruker AV-400, 376 MHz, METHANOL-d₄) 6-126.7; LCMS (Agilent Prime-6125B): m/z 419.2 [M+H]⁺; SFC (Waters UPCC with PDA Detector): 99.8% ee.

### Preparation of N-cyclopropyl-5-[(4S)-4-pyrazolo[1,5-a]pyridin-2-yl-1,4,6,7-tetrahydroimidazo[4,5-c]pyridin-5-yl]pyrazine-2-carboxamide) (Example B)

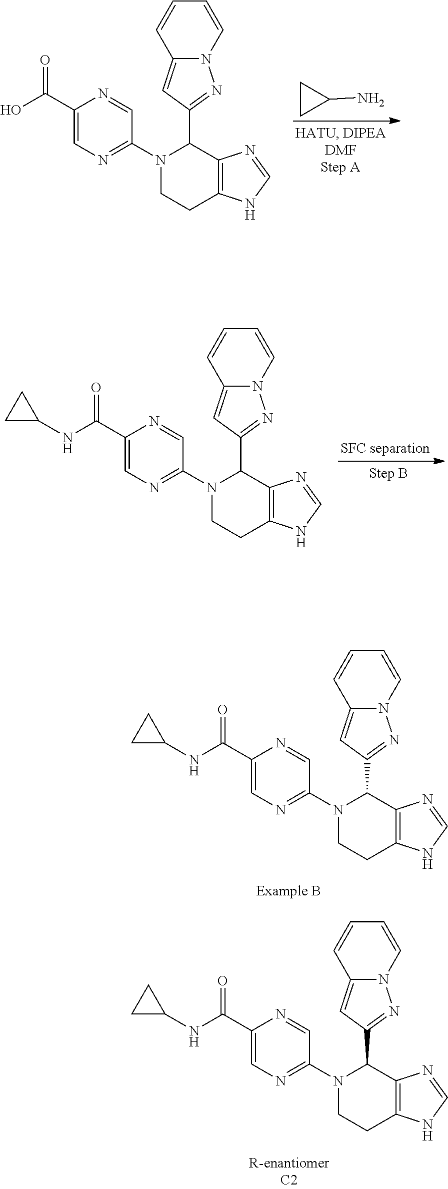

### Step A: Synthesis of N-cyclopropyl-5-[4-pyrazolo[1,5-a]pyridin-2-yl-1,4,6,7-tetrahydroimidazo[4,5-c]pyridin-5-yl]pyrazine-2-carboxamide

To a solution of 5-(4-pyrazolo[1,5-a]pyridin-2-yl-1,4,6,7-tetrahydroimidazo[4,5-c]pyridin-5-yl)pyrazine-2-carboxylic acid (2.8 g, 7.75 mmol, 1 eq) and cyclopropanamine (5.31 g, 93.0 mmol, 6.44 mL, 12 eq) in DMF (20 mL) was added HATU (5.89 g, 15.5 mmol, 2 eq) and DIPEA (3.50 g, 27.1 mmol, 4.72 mL, 3.5 eq). The reaction mixture was stirred at 25° C. for 3 hrs. DCM (50 mL) was added to the reaction mixture, and the organic layer washed with sat. NaHCO₃ solution (30 mL) and brine (30 mL). The organic layer was dried over Na₂SO₄, filtered, and concentrated to dryness. The residue was purified by flash silica gel chromatography (ISCO®; 40 g SepaFlash® Silica Flash Column, Eluent of 0-10% MeOH:DCM @40 mL/min) to give N-cyclopropyl-5-[4-pyrazolo[1,5-a]pyridin-2-yl-1,4,6,7-tetrahydroimidazo[4,5-c]pyridin-5-yl]pyrazine-2-carboxamide (1.6 g, 51.6% yield). LCMS (Shimadzu LC-20AD & MS 2020): m/z 401.2 [M+H]⁺.

### Step B: SFC Separation

N-cyclopropyl-5-[4-pyrazolo[1,5-a]pyridin-2-yl-1,4,6,7-tetrahydroimidazo[4,5-c]pyridin-5-yl]pyrazine-2-carboxamide (1.6 g, 4.00 mmol, 1 eq) was separated by SFC (Waters SFC Prep 350 System; column: DAICEL CHIRALPAK AS (250 mm*50 mm, 10 μm); mobile phase: [CO₂-EtOH(0.1% NH₃H₂O)]; B %: 25%, isocratic elution mode) to give Enantiomer #1 (508 mg, Rt=3.008 min) and Enantiomer #2 (602 mg, Rt=3.233 min).

Enantiomer #1 (Example B), N-cyclopropyl-5-[(4S)-4-pyrazolo[1,5-a]pyridin-2-yl-1,4,6,7-tetrahydroimidazo[4,5-c]pyridin-5-yl]pyrazine-2-carboxamide): ¹H NMR (Bruker AV-400, 400 MHz, METHANOL-d₄) δ 8.70 (d, 1H), 8.37-8.42 (m, 2H), 7.62 (s, 1H), 7.52 (d, 1H), 7.13 (ddd, 1H), 6.77-6.88 (m, 2H), 6.50 (s, 1H), 4.80 (br d, 1H), 3.56-3.63 (m, 1H), 2.89-2.98 (m, 1H), 2.72-2.84 (m, 2H), 0.76-0.82 (m, 2H), 0.59-0.64 (m, 2H); LCMS (Agilent Prime-6125B): m/z 401.2 [M+H]⁺; SFC (Waters UPCC with PDA Detector): 98.7% ee.

Enantiomer #2 (C2), (N-cyclopropyl-5-[(4R)-4-pyrazolo[1,5-a]pyridin-2-yl-1,4,6,7-tetrahydroimidazo[4,5-c]pyridin-5-yl]pyrazine-2-carboxamide): ¹H NMR (Bruker AV-400, 400 MHz, METHANOL-d₄) δ 8.72 (d, 1H), 8.39-8.44 (m, 2H), 7.71 (s, 1H), 7.54 (d, 1H), 7.15 (ddd, 1H), 6.79-6.89 (m, 2H), 6.52 (s, 1H), 4.80 (br d, 1H), 3.64-3.67 (m, 1H), 2.90-3.01 (m, 1H), 2.74-2.85 (m, 2H), 0.82-0.84 (m, 2H), 0.60-0.65 (m, 2H); LCMS (Agilent Prime-6125B): m/z 401.2 [M+H]⁺; SFC (Waters UPCC with PDA Detector): 98.0% ee.

### Preparation of 5-[(4S)-4-(4-fluoropyrazolo[1,5-a]pyridin-2-yl)-1,4,6,7-tetrahydroimidazo[4,5-c]pyridin-5-yl]-N-methyl-pyrazine-2-carboxamide (Example C)

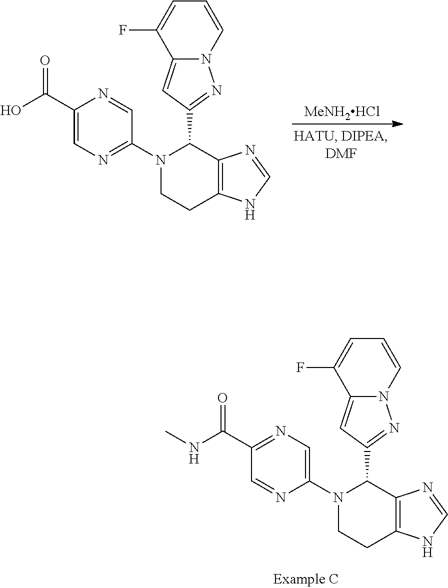 [SMILES: "MeNH2·HCl" ; O=C(O)c1cnc(N2CCc3[nH]cnc3[C@H]2c2cc3c(F)cccn3n2)cn1 ; CNC(=O)c1cnc(N2CCc3[nH]cnc3[C@H]2c2cc3c(F)cccn3n2)cn1]

To a solution of 5-[(4S)-4-(4-fluoropyrazolo[1,5-a]pyridin-2-yl)-1,4,6,7-tetrahydroimidazo[4,5-c]pyridin-5-yl]pyrazine-2-carboxylic acid (500 mg, 1.32 mmol, 1 eq) and methanamine hydrochloride (178 mg, 2.64 mmol, 2 eq) in DMF (10 mL) was added HATU (601 mg, 1.58 mmol, 1.2 eq) and DIPEA (511 mg, 3.95 mmol, 689 L, 3 eq). The reaction mixture was stirred at 25° C. for 14 hrs. Reaction progress was checked using LCMS (Shimadzu LC-20AD &MS 2020). EtOAc (60 mL) was added to the reaction mixture. The organic layer was washed with brine (20 mL×2), dried over Na₂SO₄, filtered, and concentrated to dryness. The residue was purified by flash silica gel chromatography (ISCO®; 12 g SepaFlash® Silica Flash Column, Eluent of 0~8% MeOH:DCM @30 mL/min) to give a compound (410 mg), which was combined with another 173 mg batch, and submitted for further purification using prep-HPLC (column: Boston Prime C18 150*30 mm*5 μm; mobile phase: [water(NH₃H₂O+NH₄HCO₃)-ACN]; gradient: 24%-54% B over 11 min) to give Example C, 5-[(4S)-4-(4-fluoropyrazolo[1,5-a]pyridin-2-yl)-1,4,6,7-tetrahydroimidazo[4,5-c]pyridin-5-yl]-N-methyl-pyrazine-2-carboxamide (322 mg, 55.1% yield). ¹H NMR (Bruker AV-400; 400 MHz, METHANOL-d₄) δ 8.72 (d, 1H), 8.46 (s, 1H), 8.21-8.38 (m, 1H), 7.65 (s, 1H), 6.92 (dd, 1H), 6.87 (br s, 1H), 6.72-6.83 (m, 1H), 6.66 (s, 1H), 4.83 (br d, 1H), 3.57-3.67 (m, 1H), 2.94-3.01 (m, 1H), 2.92 (s, 3H), 2.78 (dd, 1H); ¹⁹F NMR (Bruker AV-400, 376 MHz, METHANOL-d₄) δ −126.6; LCMS (Agilent Prime-6125B): m/z 393.2 [M+H]⁺; SFC (Waters UPCC with PDA Detector and QDa Detector): 99.6% ee.

### Preparation of N-tert-butyl-5-[(4S)-4-(4-fluoropyrazolo[1,5-a]pyridin-2-yl)-1,4,6,7-tetrahydroimidazo[4,5-c]pyridin-5-yl]pyrazine-2-carboxamide (Example D)

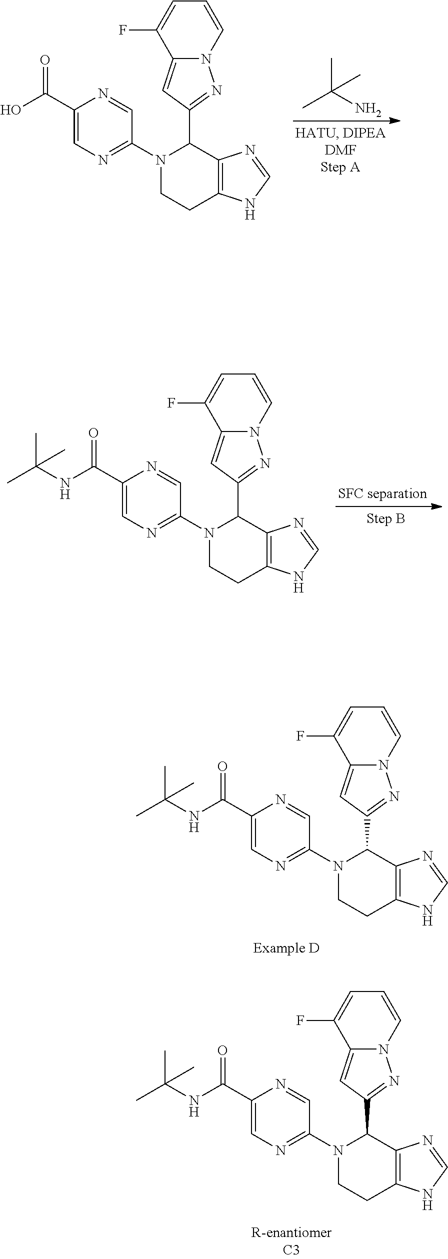

### Step A: Preparation of N-tert-butyl-5-[4-(4-fluoropyrazolo[1,5-a]pyridin-2-yl)-1,4,6,7-tetrahydroimidazo[4,5-c]pyridin-5-yl]pyrazine-2-carboxamide

To a solution of 5-[4-(4-fluoropyrazolo[1,5-a]pyridin-2-yl)-1,4,6,7-tetrahydroimidazo[4,5-c]pyridin-5-yl]pyrazine-2-carboxylic acid (500 mg, 1.32 mmol, 1 eq) and 2-methylpropan-2-amine (193 mg, 2.64 mmol, 277 L, 2 eq) in DMF (5 mL) was added HATU (601 mg, 1.58 mmol, 1.2 eq) and DIPEA (511 mg, 3.95 mmol, 689 L, 3 eq), the reaction mixture was stirred at 25° C. for 12 hrs under N₂ atmosphere. EtOAc (50 mL) was added to the reaction mixture, and the organic layer was washed by water (100 mL) and brine (50 mL), dried over Na₂SO₄, filtered, and concentrated to dryness. The residue was purified by flash silica gel chromatography (ISCO®; 20 g SepaFlash® Silica Flash Column, Eluent of 0~5% MeOH:DCM @30 mL/min) to give N-tert-butyl-5-[4-(4-fluoropyrazolo[1,5-a]pyridin-2-yl)-1,4,6,7-tetrahydroimidazo[4,5-c]pyridin-5-yl]pyrazine-2-carboxamide (540 mg, 93.4% yield). LCMS (Shimadzu LC-20AD & MS 2020): m/z 435.1 [M+H]⁺.

### Step B: SFC Separation

Two batches of N-tert-butyl-5-[4-(4-fluoropyrazolo[1,5-a]pyridin-2-yl)-1,4,6,7-tetrahydroimidazo[4,5-c]pyridin-5-yl]pyrazine-2-carboxamide were combined (collectively, 910 mg, 2.09 mmol) and separated by SFC (Waters SFC Prep 150 Mgm System; column: DAICEL CHIRALPAK IC (250 mm*30 mm, 10 μm); mobile phase: [CO₂-MeOH(0.1% NH₃H₂O)]; B %: 50%, isocratic elution mode) to give Enantiomer #1 (312 mg, Rt=1.464 min) and Enantiomer #2 (312 mg, Rt=2.258 min).

Enantiomer #1 (Example D), N-tert-butyl-5-[(4S)-4-(4-fluoropyrazolo[1,5-a]pyridin-2-yl)-1,4,6,7-tetrahydroimidazo[4,5-c]pyridin-5-yl]pyrazine-2-carboxamide: ¹H NMR (Bruker AV-400; 400 MHz, METHANOL-d₄) δ 8.70 (d, 1H), 8.44 (s, 1H), 8.32 (d, 1H), 7.65 (s, 1H), 6.93 (dd, 1H), 6.87 (s, 1H), 6.80 (td, 1H), 6.66 (s, 1H), 4.83 (br d, 1H), 3.55-3.72 (m, 1H), 2.91-3.05 (m, 1H), 2.79 (dd, 1H), 1.46 (s, 9H); ¹⁹F NMR (Bruker AV-400, 376 MHz, METHANOL-d₄) 6-126.6; LCMS (Agilent Prime-6125B): m/z 435.3 [M+H]⁺; SFC (Waters UPCC with PDA Detector and SQ Detector 2): 100% ee.

Enantiomer #2 (C3), N-tert-butyl-5-[(4R)-4-(4-fluoropyrazolo[1,5-a]pyridin-2-yl)-1,4,6,7-tetrahydroimidazo[4,5-c]pyridin-5-yl]pyrazine-2-carboxamide: ¹H NMR (Bruker AV-400; 400 MHz, METHANOL-d₄) δ 8.70 (d, 1H), 8.44 (s, 1H), 8.32 (d, 1H), 7.82 (s, 1H), 7.65 (s, 1H), 6.93 (dd, 1H), 6.87 (br s, 1H), 6.80 (td, 1H), 6.66 (s, 1H), 4.84 (br d, 1H), 3.53-3.72 (m, 1H), 2.89-3.04 (m, 1H), 2.79 (br dd, 1H), 1.46 (s, 9H); ¹⁹F NMR (Bruker AV-400, 376 MHz, METHANOL-d₄) 6-126.6; LCMS (Agilent Prime-6125B): m/z 435.3 [M+H]⁺; SFC (Waters UPCC with PDA Detector and SQ Detector 2): 100% ee.

### Preparation of (S)-5-(4-(4-fluoropyrazolo[1,5-a]pyridin-2-yl)-1,4,6,7-tetrahydro-5H-imidazo[4,5-c]pyridin-5-yl)-N-(methyl-d₃)pyrazine-2-carboxamide (Example E)

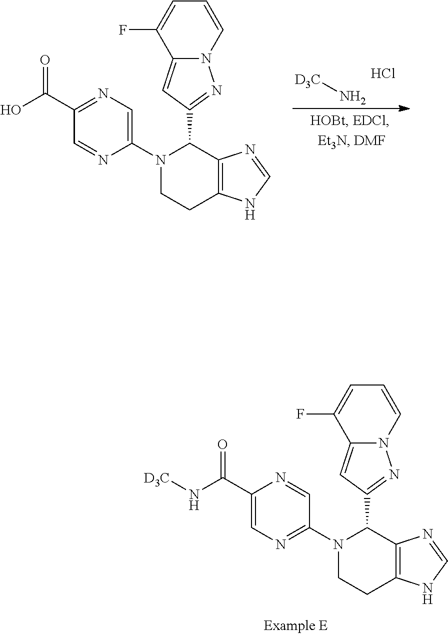 [SMILES: O=C(O)c1cnc(N2CCc3[nH]cnc3[C@H]2c2cc3c(F)cccn3n2)cn1 ; [2H]C([2H])([2H])N ; Cl ; [2H]C([2H])([2H])NC(=O)c1cnc(N2CCc3[nH]cnc3[C@H]2c2cc3c(F)cccn3n2)cn1]

To a mixture of (S)-5-(4-(4-fluoropyrazolo[1,5-a]pyridin-2-yl)-6,7-dihydro-1H-imidazo[4,5-c]pyridin-5(4H)-yl)pyrazine-2-carboxylic acid (0.1 g, 264 mol, 1 eq) in DMF (3 mL) was added HOBt (53.4 mg, 395 mol, 1.5 eq), EDCI (75.8 mg, 395 mol, 1.5 eq), trideuteriomethanamine hydrochloride (37.2 mg, 527 mol, 2 eq) and Et₃N (53.4 mg, 527 mol, 73.4 μL, 2 eq). The reaction mixture was stirred at 25° C. for 16 hrs. The reaction mixture was filtered, and the filtrate was submitted for Prep-HPLC (column: 57-Phenomenex Gemini NX C18 150×30 mm, 5 μm; mobile phase: [05-Water (10 mM NH₄HCO₃)-01-MeCN]; gradient: 18%-48% B over 11.0 min.) to give (S)-5-(4-(4-fluoropyrazolo[1,5-a]pyridin-2-yl)-1,4,6,7-tetrahydro-5H-imidazo[4,5-c]pyridin-5-yl)-N-(methyl-d₄)pyrazine-2-carboxamide (65.5 mg, 62.3% yield). ¹H NMR (Bruker AV-400, 400 MHz, METHANOL-d₄) δ 8.74 (d, 1H), 8.47 (s, 1H), 8.33 (d, 1H), 7.67 (s, 1H), 6.85-6.99 (m, 2H), 6.78-6.84 (m, 1H), 6.68 (s, 1H), 4.85 (br s, 1H), 3.59-3.69 (m, 1H), 2.94-3.03 (m, 1H), 2.80 (br dd, 1H); ¹⁹F NMR (Bruker AV-400, 376 MHz, METHANOL-d₄) 6-126.6; LCMS (Agilent Prime-6125B): m/z 396.1 [M+H]⁺; SFC (Waters UPCC with PDA Detector and SQ Detector 2): 100% ee.

### Preparation of N-methyl-5-[(4S)-4-pyrazolo[1,5-a]pyridin-2-yl-1,4,6,7-tetrahydroimidazo[4,5-c]pyridin-5-yl]pyrazine-2-carboxamide (Example I)

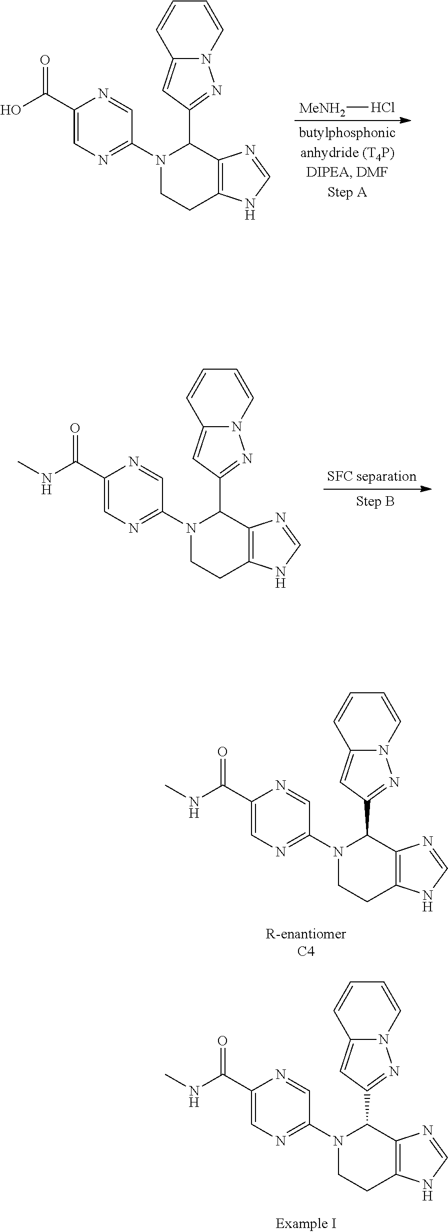

### Step A: Preparation of N-methyl-5-(4-pyrazolo[1,5-a]pyridin-2-yl-1,4,6,7-tetrahydroimidazo[4,5-c]pyridin-5-yl)pyrazine-2-carboxamide

To a solution of 5-(4-pyrazolo[1,5-a]pyridin-2-yl-1,4,6,7-tetrahydroimidazo[4,5-c]pyridin-5-yl)pyrazine-2-carboxylic acid (150 mg, 415 mol, 1 eq) and methanamine hydrochloride (140 mg, 2.08 mmol, 5 eq) in DMF (2 mL) was added DIPEA (429 mg, 3.32 mmol, 578 μL, 8 eq) and T₄P (598 mg, 830 mol, 166 μL, 50% purity, 2 eq). The mixture was stirred at 20° C. for 14 hrs. Reaction progress was checked using LCMS (Shimadzu LC-20AD &MS 2020). The reaction mixture was filtered, and the filtrate was concentrated to dryness. The residue was purified by prep-HPLC (column: 55-Boston Prime C18 150×30 mm, 5 μm; mobile phase: [H₂O(0.05% NH₃H₂O+10 mM NH₄HCO₃)-ACN]; gradient: 18%-45% B over 10.0 min) to give N-methyl-5-(4-pyrazolo[1,5-a]pyridin-2-yl-1,4,6,7-tetrahydroimidazo[4,5-c]pyridin-5-yl)pyrazine-2-carboxamide (100 mg, 63.1% yield). LCMS: m/z 375.1 [M+H]⁺.

### Step B: SFC Separation

N-methyl-5-(4-pyrazolo[1,5-a]pyridin-2-yl-1,4,6,7-tetrahydroimidazo[4,5-c]pyridin-5-yl)pyrazine-2-carboxamide (100 mg) was separated by SFC (Waters SFC Prep 150 Mgm System; column: DAICEL CHIRALPAK AD (250 mm*30 mm, 10 μm); mobile phase: [CO₂-IPA(0.1% NH₃H₂O)]; B %: 50%, isocratic elution mode) to give Enantiomer #1 (29.4 mg, Rt=1.658 min) and Enantiomer #2 (29.8 mg, Rt=2.830 min).

Enantiomer #1 (C4), N-methyl-5-[(4R)-4-pyrazolo[1,5-a]pyridin-2-yl-1,4,6,7-tetrahydroimidazo[4,5-c]pyridin-5-yl]pyrazine-2-carboxamide: ¹H NMR (Bruker AV-400, 400 MHz, METHANOL-d₄) δ 8.72 (d, 1H), 8.46 (d, 1H), 8.43 (d, 1H), 7.64 (s, 1H), 7.55 (d, 1H), 7.16 (t, 1H), 6.87 (s, 1H), 6.82 (t, 1H), 6.53 (s, 1H), 4.80-4.85 (m, 1H), 3.59-3.65 (m, 1H), 2.94-3.02 (m, 1H), 2.92 (s, 3H), 2.77 (dd, 1H); LCMS (Agilent Prime-6125B): m/z 375.1 [M+H]⁺; SFC (Waters UPCC with PDA Detector and SQ Detector 2): 100% ee.

Enantiomer #2 (Example I), N-methyl-5-[(4S)-4-pyrazolo[1,5-a]pyridin-2-yl-1,4,6,7-tetrahydroimidazo[4,5-c]pyridin-5-yl]pyrazine-2-carboxamide: ¹H NMR (Bruker AV-400, 400 MHz, METHANOL-d₄) δ 8.72 (d, 1H), 8.45 (d, 1H), 8.42 (d, 1H), 7.64 (s, 1H), 7.55 (d, 1H), 7.16 (t, 1H), 6.87 (s, 1H), 6.82 (t, 1H), 6.53 (s, 1H), 4.80-4.84 (m, 1H), 3.61-3.65 (m, 1H), 2.94-3.01 (m, 1H), 2.92 (s, 3H), 2.77 (dd, 1H); LCMS (Agilent Prime-6125B): m/z 375.1 [M+H]⁺; SFC (Waters UPCC with PDA Detector and SQ Detector 2): 99.58% ee.

### Preparation of N-tert-butyl-5-[(4S)-4-pyrazolo[1,5-a]pyridin-2-yl-1,4,6,7-tetrahydroimidazo[4,5-c]pyridin-5-yl]pyrazine-2-carboxamide (Example J)

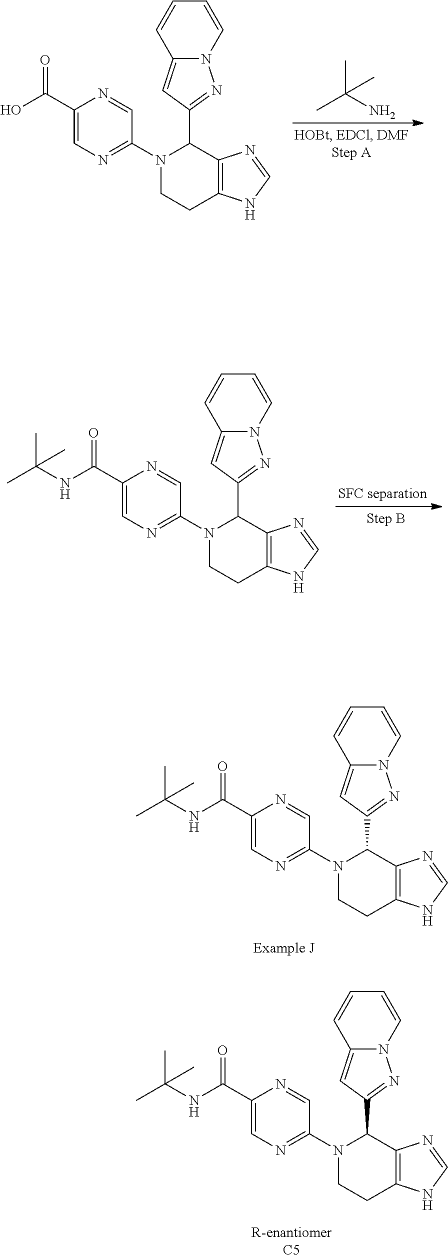

### Step A: Preparation of N-tert-butyl-5-(4-pyrazolo[1,5-a]pyridin-2-yl-1,4,6,7-tetrahydroimidazo[4,5-c]pyridin-5-yl)pyrazine-2-carboxamide

To a solution of 5-(4-pyrazolo[1,5-a]pyridin-2-yl-1,4,6,7-tetrahydroimidazo[4,5-c]pyridin-5-yl)pyrazine-2-carboxylic acid (150 mg, 415 mol, 1 eq) and 2-methylpropan-2-amine (152 mg, 2.08 mmol, 218 μL, 5 eq) in DMF (2 mL) was added EDCI (279 mg, 1.45 mmol, 3.5 eq) and HOBt (196 mg, 1.45 mmol, 3.5 eq). The reaction mixture was stirred at 20° C. for 14 hrs. The reaction mixture was filtered and concentrated under reduced pressure to dryness. The residue was purified by prep-HPLC (column: 55-Boston Prime C18 150×30 mm, 5 μm; mobile phase: [H₂O (0.05% NH₃H₂O+10 mM NH₄HCO₃)-ACN]; gradient: 32%-57% B over 10.0 min) to give N-tert-butyl-5-(4-pyrazolo[1,5-a]pyridin-2-yl-1,4,6,7-tetrahydroimidazo[4,5-c]pyridin-5-yl)pyrazine-2-carboxamide (95 mg, 53.3% yield). LCMS (Shimadzu LC-20AD &MS 2020): m/z 417.2 [M+H]⁺.

### Step B: SFC Separation

N-tert-butyl-5-(4-pyrazolo[1,5-a]pyridin-2-yl-1,4,6,7-tetrahydroimidazo[4,5-c]pyridin-5-yl)pyrazine-2-carboxamide (95 mg) was separated by SFC (Waters SFC Prep 150 Mgm System; column: DAICEL CHIRALPAK IC (250 mm*30 mm, 10 μm); mobile phase: [CO₂-EtOH(0.1% NH₃H₂O)]; B %: 55%, isocratic elution mode) to give Enantiomer #1 (27.2 mg, Rt=2.081 min) and Enantiomer #2 (25.1 mg, Rt=4.353 min).

Enantiomer #1 (Example J), N-tert-butyl-5-[(4S)-4-pyrazolo[1,5-a]pyridin-2-yl-1,4,6,7-tetrahydroimidazo[4,5-c]pyridin-5-yl]pyrazine-2-carboxamide. ¹H NMR (Bruker AV-400, 400 MHz, METHANOL-d₄) δ 8.70 (d, 1H), 8.42-8.43 (m, 2H), 7.64 (s, 1H), 7.55 (d, 1H), 7.12-7.20 (m, 1H), 6.80-6.86 (m, 2H), 6.53 (s, 1H), 4.80-4.81 (m, 1H), 3.58-3.68 (m, 1H), 2.90-3.02 (m, 1H), 2.78 (dd, 1H), 1.45 (s, 9H); LCMS (Agilent Prime-6125B): m/z 417.1 [M+H]⁺; SFC (Waters UPCC with PDA Detector and QDa Detector): 100% ee.

Enantiomer #2 (C5), N-tert-butyl-5-[(4R)-4-pyrazolo[1,5-a]pyridin-2-yl-1,4,6,7-tetrahydroimidazo[4,5-c]pyridin-5-yl]pyrazine-2-carboxamide. ¹H NMR (Bruker AV-400, 400 MHz, METHANOL-d₄) δ 8.70 (s, 1H), 8.42-8.43 (m, 2H), 7.64 (s, 1H), 7.55 (d, 1H), 7.12-7.20 (m, 1H), 6.80-6.86 (m, 2H), 6.53 (s, 1H), 4.80-4.81 (m, 1H), 3.56-3.68 (m, 1H), 2.90-3.02 (m, 1H), 2.78 (dd, 1H), 1.46 (s, 9H); LCMS (Agilent Prime-6125B): m/z 417.1 [M+H]⁺; SFC (Waters UPCC with PDA Detector and QDa Detector): 100% ee.

### Preparation of 5-[(4S)-4-pyrazolo[1,5-a]pyridin-2-yl-1,4,6,7-tetrahydroimidazo[4,5-c]pyridin-5-yl]-N-(trideuteriomethyl)pyrazine-2-carboxamide (Example K)

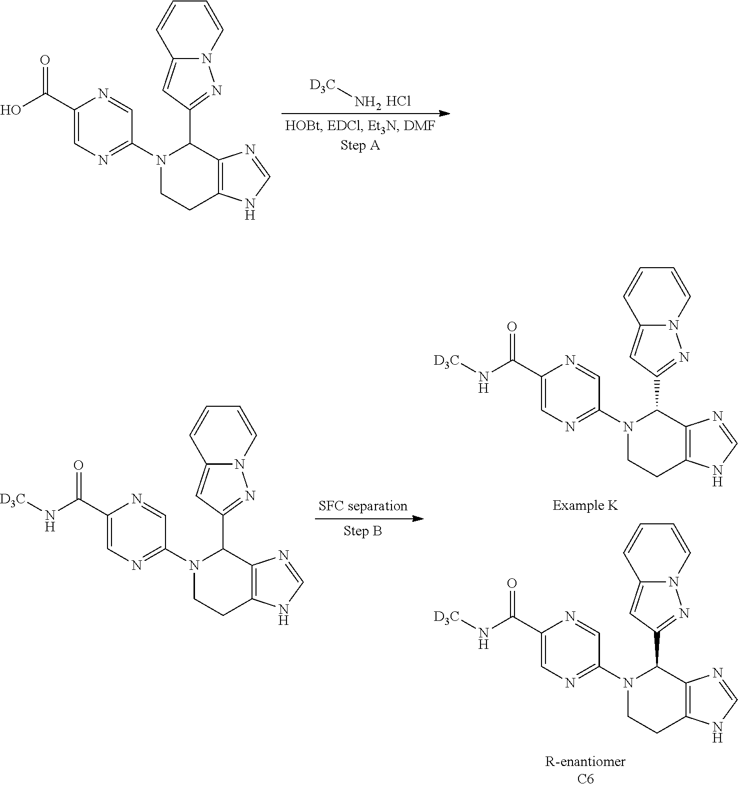 [SMILES: O=C(O)c1cnc(N2CCc3[nH]cnc3C2c2cc3ccccn3n2)cn1 ; [2H]C([2H])([2H])N ; Cl ; [2H]C([2H])([2H])NC(=O)c1cnc(N2CCc3[nH]cnc3C2c2cc3ccccn3n2)cn1 ; [2H]C([2H])([2H])NC(=O)c1cnc(N2CCc3[nH]cnc3[C@@H]2c2cc3ccccn3n2)cn1 ; [2H]C([2H])([2H])NC(=O)c1cnc(N2CCc3[nH]cnc3[C@H]2c2cc3ccccn3n2)cn1]

### Step A: Preparation of 5-(4-pyrazolo[1,5-a]pyridin-2-yl-1,4,6,7-tetrahydroimidazo[4,5-c]pyridin-5-yl)-N-(trideuteriomethyl)pyrazine-2-carboxamide

To a solution of 5-(4-pyrazolo[1,5-a]pyridin-2-yl-1,4,6,7-tetrahydroimidazo[4,5-c]pyridin-5-yl)pyrazine-2-carboxylic acid (0.2 g, 553 mol, 1 eq) in DMF (3 mL) was added trideuteriomethanamine hydrochloride (58.6 mg, 830 mol, 1.5 eq), HOBt (97.2 mg, 720 mol, 1.3 eq), EDCI (138 mg, 720 mol, 1.3 eq) and Et₃N (112 mg, 1.11 mmol, 154 μL, 2 eq). The mixture was stirred at 20° C. for 16 hrs. DCM (30 mL) was added to the reaction mixture. The organic layer was washed by H₂O (10 mL×2), dried over Na₂SO₄, filtered, and concentrated to dryness. The residue was purified by prep-HPLC (column: 55-Boston Prime C18 150×30 mm, 5 μm; mobile phase: [H₂O(0.05% NH₃H₂O+10 mM NH₄HCO₃)—CAN]; gradient: 18%-38% B over 10.0 min) to give 5-(4-pyrazolo[1,5-a]pyridine-2-yl-1,4,6,7-tetrahydroimidazo[4,5-c]pyridine-5-yl)-N-(trideuteriomethyl)pyrazine-2-carboxamide (92 mg, 43.6% yield). LCMS (Shimadzu LC-20AD &MS 2020): m/z 378.1 [M+H]⁺.

### Step 2: SFC Separation

5-(4-pyrazolo[1,5-a]pyridin-2-yl-1,4,6,7-tetrahydroimidazo[4,5-c]pyridin-5-yl)-N-(trideuteriomethyl)pyrazine-2-carboxamide (92 mg) was separated by SFC (Waters SFC Prep 150 Mgm System; column: DAICEL CHIRALPAK AD (250 mm*30 mm, 10 μm); mobile phase: [CO₂—IPA(0.1% NH₃H₂O)]; B %: 50%, isocratic elution mode) to give Enantiomer #1 (30 mg, Rt=1.345 min) and Enantiomer #2 (32.2 mg, Rt=2.270 min).

Enantiomer #1 (C6), 5-[(4R)-4-pyrazolo[1,5-a]pyridin-2-yl-1,4,6,7-tetrahydroimidazo[4,5-c]pyridin-5-yl]-N-(trideuteriomethyl)pyrazine-2-carboxamide: ¹H NMR (Bruker AV-400; 400 MHz, METHANOL-d₄) δ 8.61 (d, 1H), 8.27-8.37 (m, 2H), 7.53 (s, 1H), 7.43 (d, 1H), 7.03-7.06 (m, 1H), 6.65-6.80 (m, 2H), 6.42 (s, 1H), 4.71 (br dd, 1H), 3.48-3.53 (m, 1H), 2.77-2.92 (m, 1H), 2.66 (dd, 1H); LCMS (Agilent Prime-6125B): m/z 378.2 [M+H]⁺; SFC (Waters UPCC with PDA Detector and QDa Detector): 99.9% ee.

Enantiomer #2 (Example K), 5-[(4S)-4-pyrazolo[1,5-a]pyridin-2-yl-1,4,6,7-tetrahydroimidazo[4,5-c]pyridin-5-yl]-N-(trideuteriomethyl)pyrazine-2-carboxamide: ¹H NMR (Bruker AV-400; 400 MHz, METHANOL-d₄) δ 8.61 (d, 1H), 8.26-8.36 (m, 2H), 7.53 (s, 1H), 7.43 (d, 1H), 7.02-7.06 (m, 1H), 6.63-6.80 (m, 2H), 6.41 (s, 1H), 4.70 (br dd, 1H), 3.47-3.53 (m, 1H), 2.78-2.91 (m, 1H), 2.65 (dd, 1H); LCMS (Agilent Prime-6125B): m/z 378.2 [M+H]⁺; SFC (Waters UPCC with PDA Detector and QDa Detector): 99.3% ee.

### Preparation of (S)-5-(4-(4-fluoropyrazolo[1,5-a]pyridin-2-yl)-1,4,6,7-tetrahydro-5H-imidazo[4,5-c]pyridin-5-yl)-N-(propan-2-yl-d₇)pyrazine-2-carboxamide (Example G) According to General Scheme 3

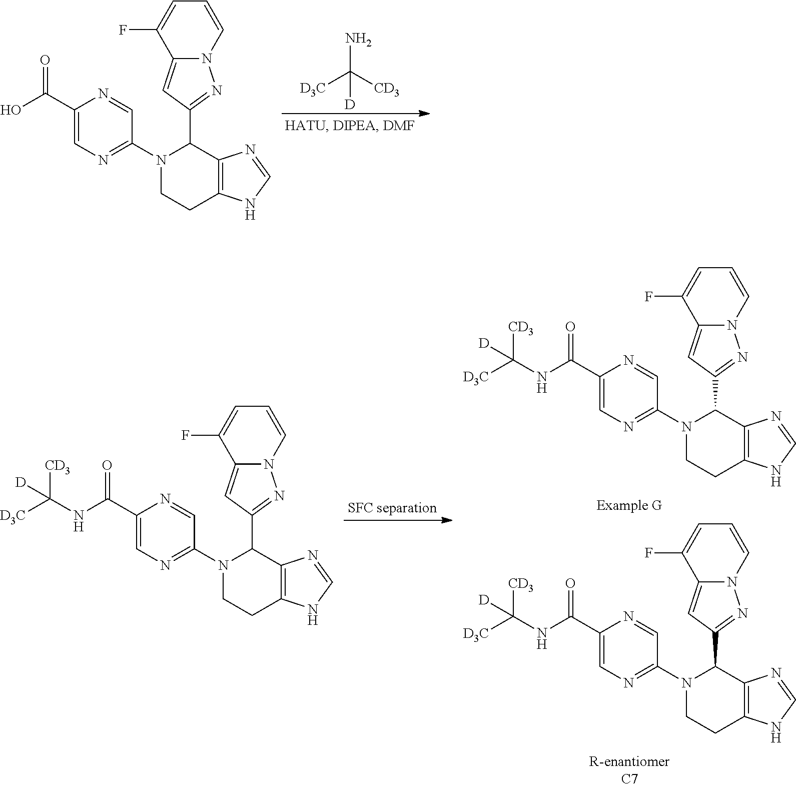 [SMILES: O=C(O)c1cnc(N2CCc3[nH]cnc3C2c2cc3c(F)cccn3n2)cn1 ; [2H]C([2H])([2H])C([2H])(N)C([2H])([2H])[2H] ; [2H]C([2H])([2H])C([2H])(NC(=O)c1cnc(N2CCc3[nH]cnc3C2c2cc3c(F)cccn3n2)cn1)C([2H])([2H])[2H] ; [2H]C([2H])([2H])C([2H])(NC(=O)c1cnc(N2CCc3[nH]cnc3[C@@H]2c2cc3c(F)cccn3n2)cn1)C([2H])([2H])[2H] ; [2H]C([2H])([2H])C([2H])(NC(=O)c1cnc(N2CCc3[nH]cnc3[C@H]2c2cc3c(F)cccn3n2)cn1)C([2H])([2H])[2H]]

### Step A: Preparation of 5-(4-(4-fluoropyrazolo[1,5-a]pyridin-2-yl)-1,4,6,7-tetrahydro-5H-imidazo[4,5-c]pyridin-5-yl)-N-(propan-2-yl-d₇)pyrazine-2-carboxamide

5-(4-(4-fluoropyrazolo[1,5-a]pyridin-2-yl)-1,4,6,7-tetrahydro-5H-imidazo[4,5-c]pyridin-5-yl)pyrazine-2-carboxylic acid (Lithium salt, 147 mg, 1 eq, 381 mol) was dissolved in DMF (2.0 mL), then HATU (304 mg, 2.1 eq, 799 mol) and diisopropylethylamine (103 mg, 138 μL, 2.1 eq, 799 mol) were added. The reaction mixture was stirred at rt for 10 mins, then propan-d₇-2-amine (27.7 mg, 35.7 L, 1.1 eq, 419 mol) was added. The reaction mixture was stirred at rt for 3 hrs. Water (20 mL) was added to the reaction mixture, and the organic portion extracted with EtOAc (20 mL×3). The combined organic layer was washed with brine, dried over Na₂SO₄, filtered, and concentrated to dryness. The residue was purified by column chromatography (Biotage 10 gr Sfar column, 18 mL/min, 0-100% EtOAc in heptane, then 0-15% EtOH in EtOAc) to give 5-(4-(4-fluoropyrazolo[1,5-a]pyridin-2-yl)-1,4,6,7-tetrahydro-5H-imidazo[4,5-c]pyridin-5-yl)-N-(propan-2-yl-d₇)pyrazine-2-carboxamide (94 mg, 58% yield). ¹H NMR (400 MHz, METHANOL-d₄) δ 8.73 (t, 1H), 8.47 (d, 1H), 8.32 (dd, 1H), 7.66 (d, 1H), 6.95-6.88 (m, 2H), 6.82-6.77 (m, 1H), 6.66 (s, 1H), 4.87 (d, 1H), 3.67-3.59 (m, 1H), 2.97 (dd, 2H), 2.82-2.76 (m, 1H); LCMS: m/z 428.2 [M+H]⁺.

### Step B: SFC Separation

5-(4-(4-fluoropyrazolo[1,5-a]pyridin-2-yl)-1,4,6,7-tetrahydro-5H-imidazo[4,5-c]pyridin-5-yl)-N-(propan-2-yl-d₇)pyrazine-2-carboxamide (94 mg) was separated by SFC (Waters SFC Prep 150 Mgm System; column: ChiralPak AD, 250×30 mm I.D., 10 μm; mobile phase: [CO₂—IPA(0.1% NH₃H₂O)]; B %: 45%, isocratic elution mode) to give Enantiomer #1 (20.9 mg, Rt=0.766 min) and Enantiomer #2 (22.7 mg, Rt=2.228 min).

Enantiomer #1 (C7), (R)-5-(4-(4-fluoropyrazolo[1,5-a]pyridin-2-yl)-1,4,6,7-tetrahydro-5H-imidazo[4,5-c]pyridin-5-yl)-N-(propan-2-yl-d₇)pyrazine-2-carboxamide: ¹H NMR (Bruker AV-400, 400 MHz, METHANOL-d₄) δ 8.72 (d, 1H), 8.47 (d, 1H), 8.32 (d, 1H), 7.67 (s, 1H), 6.85-6.99 (m, 2H), 6.80 (td, 1H), 6.67 (s, 1H), 4.84 (br d, 1H), 3.63 (ddd, 1H), 2.91-3.05 (m, 1H), 2.79 (dd, 1H); ¹⁹F NMR (Bruker AV-400, 376 MHz, METHANOL-d₄) 6-126.6; LCMS (Shimadzu LC-30AD &MS 2020): m/z 428.2 [M+H]⁺; SFC (Waters UPCC with PDA Detector): 100% ee.

Enantiomer #2 (Example G), (S)-5-(4-(4-fluoropyrazolo[1,5-a]pyridin-2-yl)-1,4,6,7-tetrahydro-5H-imidazo[4,5-c]pyridin-5-yl)-N-(propan-2-yl-d₇)pyrazine-2-carboxamide: ¹H NMR (Bruker AV-400, 400 MHz, METHANOL-d₄) δ 8.73 (d, 1H), 8.47 (d, 1H), 8.32 (d, 1H), 7.66 (s, 1H), 6.85-7.00 (m, 2H), 6.80 (td, 1H), 6.67 (s, 1H), 4.83 (br s, 1H), 3.63 (ddd, 1H), 2.90-3.06 (m, 1H), 2.79 (dd, 1H); ¹⁹F NMR (Bruker AV-400, 376 MHz, METHANOL-d₄) 6-126.6; LCMS (Shimadzu LC-30AD &MS 2020): m/z 428.3 [M+H]⁺; SFC (Waters UPCC with PDA Detector): 100% ee.

### Preparation of (S)-N-(2-(methyl-d₃)propan-2-yl-1,1,1,3,3,3-d₆)-5-(4-(pyrazolo[1,5-a]pyridin-2-yl)-1,4,6,7-tetrahydro-5H-imidazo[4,5-c]pyridin-5-yl)pyrazine-2-carboxamide (Example N) According to General Scheme 3

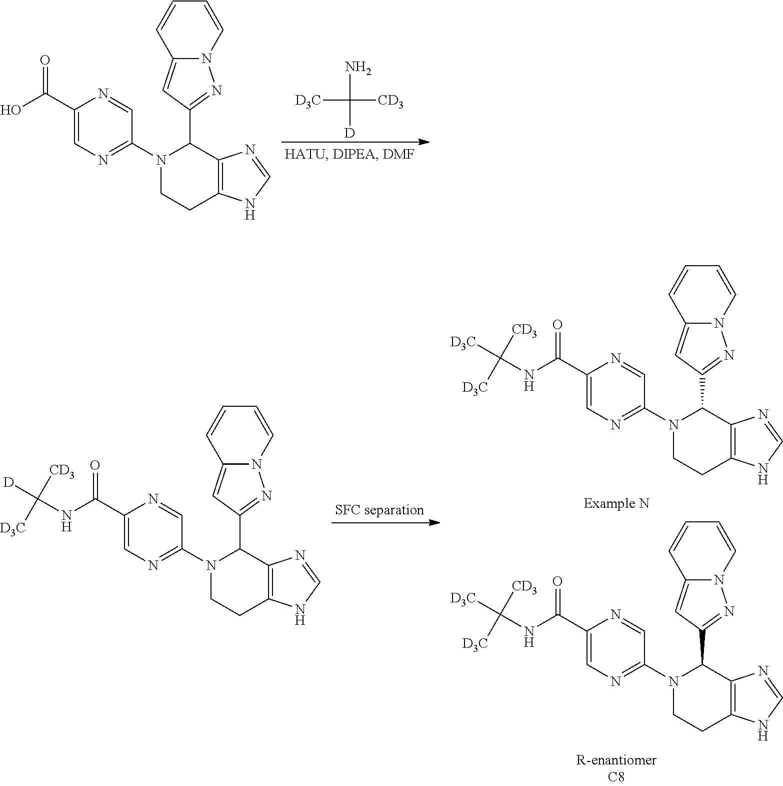 [SMILES: O=C(O)c1cnc(N2CCc3[nH]cnc3C2c2cc3ccccn3n2)cn1 ; [2H]C([2H])([2H])C([2H])(N)C([2H])([2H])[2H] ; [2H]C([2H])([2H])C([2H])(NC(=O)c1cnc(N2CCc3[nH]cnc3C2c2cc3ccccn3n2)cn1)C([2H])([2H])[2H] ; [2H]C([2H])([2H])C(NC(=O)c1cnc(N2CCc3[nH]cnc3[C@@H]2c2cc3ccccn3n2)cn1)(C([2H])([2H])[2H])C([2H])([2H])[2H] ; [2H]C([2H])([2H])C(NC(=O)c1cnc(N2CCc3[nH]cnc3[C@H]2c2cc3ccccn3n2)cn1)(C([2H])([2H])[2H])C([2H])([2H])[2H]]

### Step A: Preparation of N-(2-(methyl-d₃)propan-2-yl-1,1,1,3,3,3-d₆)-5-(4-(pyrazolo[1,5-a]pyridin-2-yl)-1,4,6,7-tetrahydro-5H-imidazo[4,5-c]pyridin-5-yl)pyrazine-2-carboxamide

5-(4-(pyrazolo[1,5-a]pyridin-2-yl)-1,4,6,7-tetrahydro-5H-imidazo[4,5-c]pyridin-5-yl)pyrazine-2-carboxylic acid (lithium salt, 175 mg, 1 eq, 475 mol) was dissolved in DMF (2.5 mL), then HATU (379 mg, 2.1 eq, 998 mol) and diisopropylethylamine (129 mg, 172 μL, 2.1 eq, 998 mol) were added. The reaction mixture was stirred at rt for 10 mins, and then 2-(²H₃)Methyl(²H₆)propan-2-amine (43.0 mg, 51.6 μL, 1.1 eq, 523 mol) was added. The reaction mixture was stirred at rt for overnight. Water (20 mL) was added to the reaction mixture, and the organic portion was extracted with EtOAc (20 mL×3). The combined organic layer was washed with brine, was dried over Na₂SO₄, filtered, and concentrated to dryness. The residue was purified by column chromatography (Biotage 10 gr Sfar column, 18 mL/min, 0-100% EtOAc in heptane, then 0-15% EtOH in EtOAc) to give N-(2-(methyl-d₃)propan-2-yl-1,1,1,3,3,3-d6)-5-(4-(pyrazolo[1,5-a]pyridin-2-yl)-1,4,6,7-tetrahydro-5H-imidazo[4,5-c]pyridin-5-yl)pyrazine-2-carboxamide (116 mg, 57.4% yield). ¹H NMR (400 MHz, METHANOL-d₄) δ 8.70 (d, 1H), 8.44-8.41 (m, 2H), 7.64 (s, 1H), 7.54 (dt, 1H), 7.15 (ddd, 1H), 6.86 (s, 1H), 6.81 (td, 1H), 6.52 (s, 1H), 4.85 (m, 1H), 3.66-3.58 (m, 1H), 2.99-2.92 (m, 1H), 2.81-2.75 (m, 1H); LCMS: m/z 426.2 [M+H]⁺.

### Step B: SFC Separation

N-(2-(methyl-d₃)propan-2-yl-1,1,1,3,3,3-d₆)-5-(4-(pyrazolo[1,5-a]pyridin-2-yl)-1,4,6,7-tetrahydro-5H-imidazo[4,5-c]pyridin-5-yl)pyrazine-2-carboxamide (116 mg) was separated by SFC (Waters SFC Prep 150 Mgm System; column: ChiralPakAD, 250×30 mm I.D., 10 μm; mobile phase: [CO₂-MeOH(0.1% NH₃H₂O)]; B %: 45%, isocratic elution mode) to give Enantiomer #1 (23.6 mg, Rt=1.050 min) and Enantiomer #2 (24.2 mg, Rt=2.631 min).

Enantiomer #1 (Example N), (S)-N-(2-(methyl-d₃)propan-2-yl-1,1,1,3,3,3-d₆)-5-(4-(pyrazolo[1,5-a]pyridin-2-yl)-1,4,6,7-tetrahydro-5H-imidazo[4,5-c]pyridin-5-yl)pyrazine-2-carboxamide: ¹H NMR (Bruker AV-400; 400 MHz, METHANOL-d₄) δ 8.70 (d, 1H), 8.37-8.51 (m, 2H), 7.80 (s, 1H), 7.64 (s, 1H), 7.55 (d, 1H), 7.14-7.18 (m, 1H), 6.86 (s, 1H), 6.82 (td, 1H), 6.53 (s, 1H), 4.82 (br dd, 1H), 3.59-3.65 (m, 1H), 2.89-3.02 (m, 1H), 2.77 (dd, 1H); LCMS (Shimadzu LC-30AD & MS 2020): m/z 426.3 [M+H]⁺; SFC (Waters UPCC with PDA Detector): 100% ee.

Enantiomer #2 (C8), (R)-N-(2-(methyl-d₃)propan-2-yl-1,1,1,3,3,3-d₆)-5-(4-(pyrazolo[1,5-a]pyridin-2-yl)-1,4,6,7-tetrahydro-5H-imidazo[4,5-c]pyridin-5-yl)pyrazine-2-carboxamide: ¹H NMR (Bruker AV-400; 400 MHz, METHANOL-d₄) δ 8.69 (d, 1H), 8.36-8.45 (m, 2H), 7.79 (s, 1H), 7.64 (s, 1H), 7.54 (d, 1H), 7.13-7.17 (m, 1H), 6.86 (s, 1H), 6.81 (td, 1H), 6.52 (s, 1H), 4.82 (br dd, 1H), 3.59-3.64 (m, 1H), 2.90-3.03 (m, 1H), 2.77 (dd, 1H); LCMS (Shimadzu LC-30AD & MS 2020): m/z 426.3 [M+H]⁺; SFC (Waters UPCC with PDA Detector): 100% ee.

### Preparation of (S)-5-(4-(4-fluoropyrazolo[1,5-a]pyridin-2-yl)-1,4,6,7-tetrahydro-5H-imidazo[4,5-c]pyridin-5-yl)-N-(2-(methyl-d₃)propan-2-yl-1,1,1,3,3,3-d₆)pyrazine-2-carboxamide (Example H) According to General Scheme 3

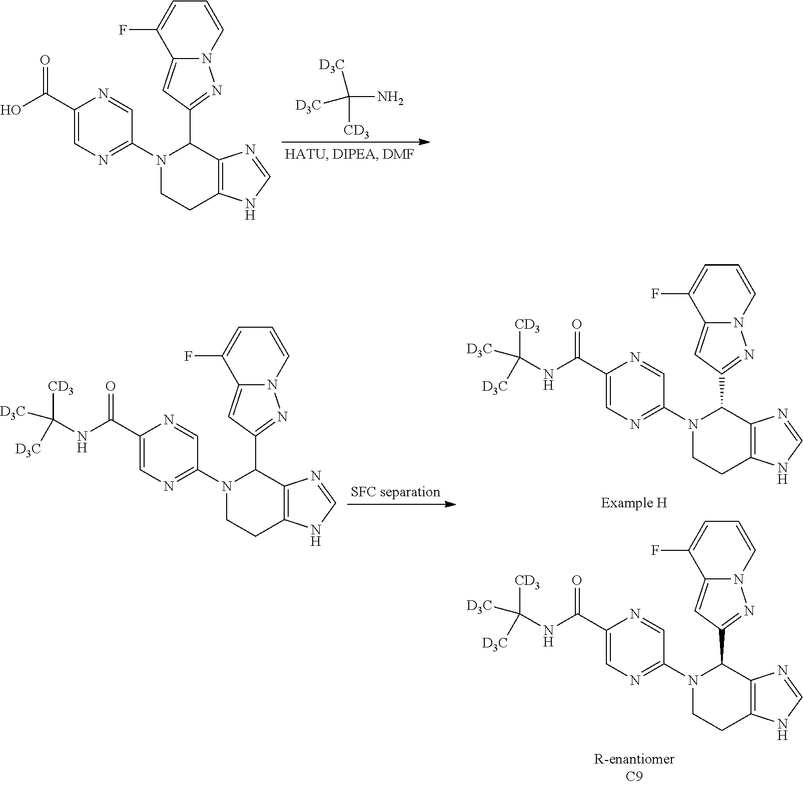 [SMILES: O=C(O)c1cnc(N2CCc3[nH]cnc3C2c2cc3c(F)cccn3n2)cn1 ; [2H]C([2H])([2H])C(N)(C([2H])([2H])[2H])C([2H])([2H])[2H] ; [2H]C([2H])([2H])C(NC(=O)c1cnc(N2CCc3[nH]cnc3C2c2cc3c(F)cccn3n2)cn1)(C([2H])([2H])[2H])C([2H])([2H])[2H] ; [2H]C([2H])([2H])C(NC(=O)c1cnc(N2CCc3[nH]cnc3[C@@H]2c2cc3c(F)cccn3n2)cn1)(C([2H])([2H])[2H])C([2H])([2H])[2H] ; [2H]C([2H])([2H])C(NC(=O)c1cnc(N2CCc3[nH]cnc3[C@H]2c2cc3c(F)cccn3n2)cn1)(C([2H])([2H])[2H])C([2H])([2H])[2H]]

Example H and its R-enantiomer C9 were prepared in accordance with the above scheme. SFC Separation Method: Waters SFC Prep 150 Mgm System; column: ChiralPak IC, 250×30 mm I.D., 10 μm; mobile phase: [CO₂-EtOH:MeCN (7:3)]; B %: 50%; 120 mL/min; isocratic elution mode.

Enantiomer #1 (Example H), (S)-5-(4-(4-fluoropyrazolo[1,5-a]pyridin-2-yl)-1,4,6,7-tetrahydro-5H-imidazo[4,5-c]pyridin-5-yl)-N-(2-(methyl-d₃)propan-2-yl-1,1,1,3,3,3-d₆)pyrazine-2-carboxamide: ¹H NMR (Bruker AV-400; 400 MHz, METHANOL-d₄) δ 8.71 (d, 1H), 8.45 (d, 1H), 8.33 (d, 1H), 7.81 (s, 1H), 7.65 (s, 1H), 6.75-6.98 (m, 3H), 6.67 (s, 1H), 4.84 (br d, 1H), 3.60-3.65 (m, 1H), 2.90-3.06 (m, 1H), 2.79 (dd, 1H); LCMS (Shimadzu LC-30AD & MS 2020): m/z 444.1 [M+H]; SFC (Waters UPCC with PDA Detector): 100% ee; Rt=2.144 min.

Enantiomer #2 (C9), (R)-5-(4-(4-fluoropyrazolo[1,5-a]pyridin-2-yl)-1,4,6,7-tetrahydro-5H-imidazo[4,5-c]pyridin-5-yl)-N-(2-(methyl-d₃)propan-2-yl-1,1,1,3,3,3-d₆)pyrazine-2-carboxamide: ¹H NMR (Bruker AV-400; 400 MHz, METHANOL-d₄) δ 8.70 (d, 1H), 8.45 (d, 1H), 8.33 (d, 1H), 7.81 (s, 1H), 7.65 (s, 1H), 6.75-7.01 (m, 3H), 6.67 (s, 1H), 4.84 (br d, 1H), 3.59-3.66 (m, 1H), 2.90-3.05 (m, 1H), 2.79 (dd, 1H); LCMS (Shimadzu LC-30AD & MS 2020): m/z 444.2 [M+H]; SFC (Waters UPCC with PDA Detector): 98.6% ee; Rt=4.674 min.

### Preparation of (S)-N-(ethyl-d₅)-5-(4-(4-fluoropyrazolo[1,5-a]pyridin-2-yl)-1,4,6,7-tetrahydro-5H-imidazo[4,5-c]pyridin-5-yl)pyrazine-2-carboxamide (Example F) According to General Scheme 3

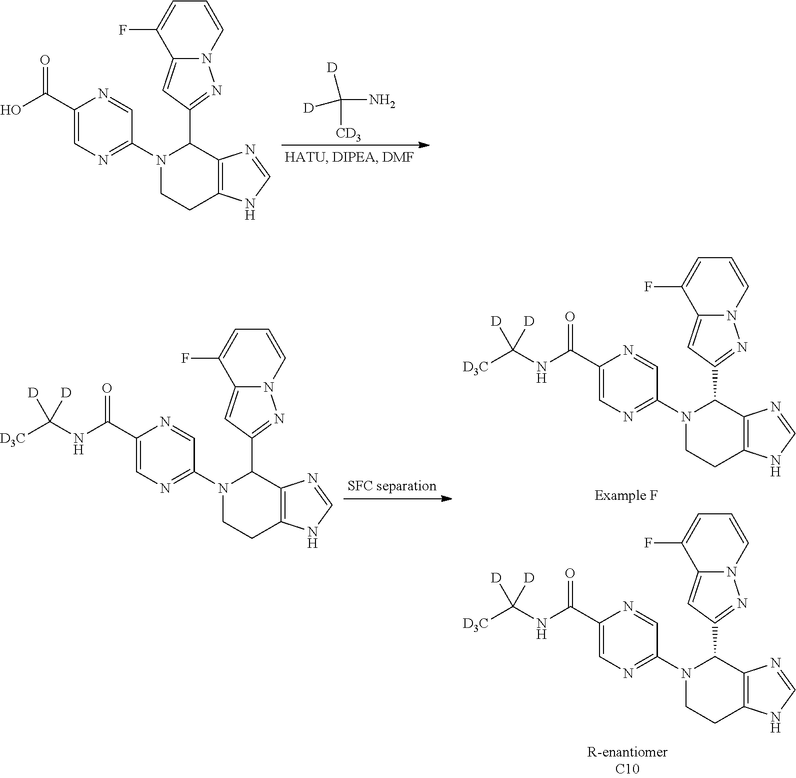 [SMILES: O=C(O)c1cnc(N2CCc3[nH]cnc3C2c2cc3c(F)cccn3n2)cn1 ; [2H]C([2H])([2H])C([2H])([2H])N ; [2H]C([2H])([2H])C([2H])([2H])NC(=O)c1cnc(N2CCc3[nH]cnc3C2c2cc3c(F)cccn3n2)cn1 ; [2H]C([2H])([2H])C([2H])([2H])NC(=O)c1cnc(N2CCc3[nH]cnc3[C@H]2c2cc3c(F)cccn3n2)cn1 ; [2H]C([2H])([2H])C([2H])([2H])NC(=O)c1cnc(N2CCc3[nH]cnc3[C@H]2c2cc3c(F)cccn3n2)cn1]

Example F and its R-enantiomer C10 were prepared in accordance with the above scheme. SFC Separation Method: Waters SFC Prep 150 Mgm System; column: ChiralPak AD, 250×30 mm I.D., 10 μm; mobile phase: [CO₂-EtOH]; B %: 40%; 120 mL/min; isocratic elution mode.

Enantiomer #1 (C10), (R)-N-(ethyl-d₅)-5-(4-(4-fluoropyrazolo[1,5-a]pyridin-2-yl)-1,4,6,7-tetrahydro-5H-imidazo[4,5-c]pyridin-5-yl)pyrazine-2-carboxamide: ¹H NMR (Bruker AV-400; 400 MHz, METHANOL-d₄) δ 8.75 (d, 1H), 8.49 (d, 1H), 8.34 (d, 1H), 7.68 (s, 1H), 6.88-7.00 (m, 2H), 6.82 (td, 1H), 6.69 (s, 1H), 4.85 (br s, 1H), 3.58-3.72 (m, 1H), 2.90-3.06 (m, 1H), 2.81 (dd, 1H); LCMS (Shimadzu LC-30AD & MS 2020): m/z 412.2 [M+H]; SFC (Waters UPCC with PDA Detector): 100% ee; Rt=0.974 min.

Enantiomer #2 (Example F), (S)-N-(ethyl-d₅)-5-(4-(4-fluoropyrazolo[1,5-a]pyridin-2-yl)-1,4,6,7-tetrahydro-5H-imidazo[4,5-c]pyridin-5-yl)pyrazine-2-carboxamide: ¹H NMR (Bruker AV-400; 400 MHz, METHANOL-d₄) δ 8.74 (d, 1H), 8.48 (d, 1H), 8.33 (d, 1H), 7.67 (s, 1H), 6.87-7.02 (m, 2H), 6.81 (td, 1H), 6.68 (s, 1H), 4.85 (br d, 1H), 3.58-3.70 (m, 1H), 2.92-3.05 (m, 1H), 2.80 (dd, 1H); LCMS (Shimadzu LC-30AD & MS 2020): m/z 412.2 [M+H]; SFC (Waters UPCC with PDA Detector): 99.9% ee; Rt=1.584 min.

### Preparation of (S)-N-(propan-2-yl-d₇)-5-(4-(pyrazolo[1,5-a]pyridin-2-yl)-1,4,6,7-tetrahydro-5H-imidazo[4,5-c]pyridin-5-yl)pyrazine-2-carboxamide (Example M) According to General Scheme 3

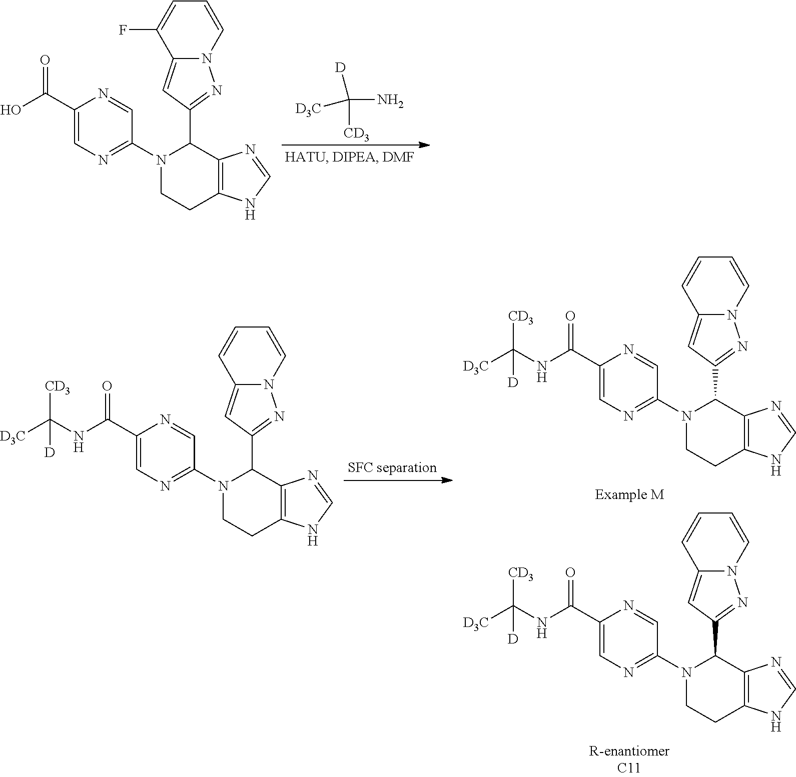 [SMILES: O=C(O)c1cnc(N2CCc3[nH]cnc3C2c2cc3c(F)cccn3n2)cn1 ; [2H]C([2H])([2H])C([2H])(N)C([2H])([2H])[2H] ; [2H]C([2H])([2H])C([2H])(NC(=O)c1cnc(N2CCc3[nH]cnc3C2c2cc3ccccn3n2)cn1)C([2H])([2H])[2H] ; [2H]C([2H])([2H])C([2H])(NC(=O)c1cnc(N2CCc3[nH]cnc3[C@@H]2c2cc3ccccn3n2)cn1)C([2H])([2H])[2H] ; [2H]C([2H])([2H])C([2H])(NC(=O)c1cnc(N2CCc3[nH]cnc3[C@H]2c2cc3ccccn3n2)cn1)C([2H])([2H])[2H]]

Example M and its R-enantiomer C11 were prepared in accordance with the above scheme. SFC Separation Method: Waters SFC Prep 150 Mgm System; column: ChiralPak AD, 250×30 mm I.D., 10 μm; mobile phase: [CO₂—Isopropanol]; B %: 45%; 120 mL/min; isocratic elution mode.

Enantiomer #1 (Example M), (S)-N-(propan-2-yl-d₇)-5-(4-(pyrazolo[1,5-a]pyridin-2-yl)-1,4,6,7-tetrahydro-5H-imidazo[4,5-c]pyridin-5-yl)pyrazine-2-carboxamide: ¹H NMR (Bruker AV-400; 400 MHz, METHANOL-d₄) δ 8.72 (s, 1H), 8.41-8.48 (m, 2H), 7.64 (s, 1H), 7.55 (d, 1H), 7.14-7.18 (m, 1H), 6.87 (s, 1H), 6.80-6.84 (m, 1H), 6.53 (s, 1H), 4.82-4.86 (m, 1H), 3.59-3.66 (m, 1H), 2.91-3.03 (m, 1H), 2.78 (dd, 1H); LCMS (Shimadzu LC-30AD & MS 2020): m/z 410.3 [M+H]; SFC (Waters UPCC with PDA Detector): 100% ee; Rt=0.766 min.

Enantiomer #2 (C11), (R)-N-(propan-2-yl-d₇)-5-(4-(pyrazolo[1,5-a]pyridin-2-yl)-1,4,6,7-tetrahydro-5H-imidazo[4,5-c]pyridin-5-yl)pyrazine-2-carboxamide: ¹H NMR (Bruker AV-400; 400 MHz, METHANOL-d₄) δ 8.72 (s, 1H), 8.41-8.49 (m, 2H), 7.64 (s, 1H), 7.55 (dd, 1H), 7.14-7.18 (m, 1H), 6.87 (s, 1H), 6.82 (t, 1H), 6.53 (s, 1H), 4.81-4.86 (m, 1H), 3.59-3.65 (m, 1H), 2.91-3.02 (m, 1H), 2.78 (dd, 1H); LCMS (Shimadzu LC-30AD & MS 2020): m/z 410.2 [M+H]⁺; SFC (Waters UPCC with PDA Detector): 99.7% ee; Rt=2.228 min.

### Preparation of (S)-N-(ethyl-d₅)-5-(4-(pyrazolo[1,5-a]pyridin-2-yl)-1,4,6,7-tetrahydro-5H-imidazo[4,5-c]pyridin-5-yl)pyrazine-2-carboxamide (Example L) According to General Scheme 3

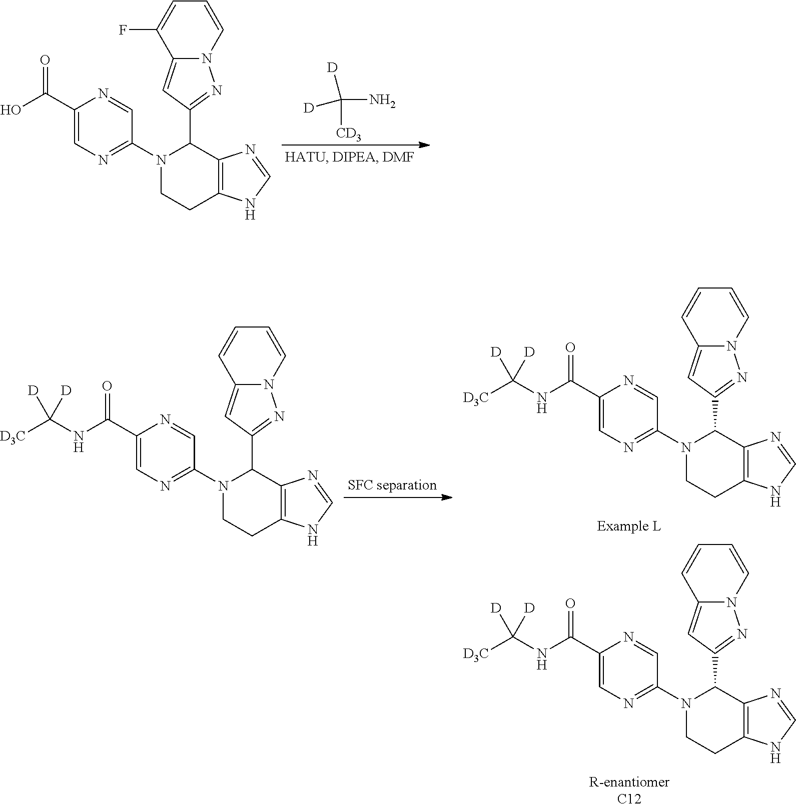 [SMILES: O=C(O)c1cnc(N2CCc3[nH]cnc3C2c2cc3c(F)cccn3n2)cn1 ; [2H]C([2H])([2H])C([2H])([2H])N ; [2H]C([2H])([2H])C([2H])([2H])NC(=O)c1cnc(N2CCc3[nH]cnc3C2c2cc3ccccn3n2)cn1 ; [2H]C([2H])([2H])C([2H])([2H])NC(=O)c1cnc(N2CCc3[nH]cnc3[C@H]2c2cc3ccccn3n2)cn1 ; [2H]C([2H])([2H])C([2H])([2H])NC(=O)c1cnc(N2CCc3[nH]cnc3[C@H]2c2cc3ccccn3n2)cn1]

Example L and its R-enantiomer C12 were prepared in accordance with the above scheme. SFC Separation Method: Waters SFC Prep 150 Mgm System; column: ChiralPak AD, 250×30 mm I.D., 10 μm; mobile phase: [CO₂-Methanol (0.1% NH3H2O)]; B %: 40%; 120 mL/min; isocratic elution mode.

Enantiomer #1 (Example L), (S)-N-(ethyl-d₅)-5-(4-(pyrazolo[1,5-a]pyridin-2-yl)-1,4,6,7-tetrahydro-5H-imidazo[4,5-c]pyridin-5-yl)pyrazine-2-carboxamide: ¹H NMR (Bruker AV-400; 400 MHz, METHANOL-d₄) δ 8.73 (d, 1H), 8.38-8.49 (m, 2H), 7.65 (s, 1H), 7.51-7.59 (m, 1H), 7.16 (ddd, 1H), 6.77-6.92 (m, 2H), 6.54 (s, 1H), 4.83 (br dd, 1H), 3.63 (ddd, 1H), 2.92-3.03 (m, 1H), 2.78 (dd, 1H); LCMS (Shimadzu LC-30AD & MS 2020): m/z 394.3 [M+H]; SFC (Waters UPCC with PDA Detector): 100% ee; Rt=0.649 min.

Enantiomer #2 (C12), (R)-N-(ethyl-d₅)-5-(4-(pyrazolo[1,5-a]pyridin-2-yl)-1,4,6,7-tetrahydro-5H-imidazo[4,5-c]pyridin-5-yl)pyrazine-2-carboxamide: ¹H NMR (Bruker AV-400; 400 MHz, METHANOL-d₄) δ 8.73 (d, 1H), 8.37-8.49 (m, 2H), 7.66 (s, 1H), 7.53 (d, 1H), 7.14 (ddd, 1H), 6.76-6.93 (m, 2H), 6.52 (s, 1H), 4.82 (br dd, 1H), 3.56-3.68 (m, 1H), 2.90-3.02 (m, 1H), 2.77 (dd, 1H); LCMS (Shimadzu LC-30AD & MS 2020): m/z 394.3 [M+H]; SFC (Waters UPCC with PDA Detector): 99.6% ee; Rt=1.064 min.

### Biological Assays and Data

### Cell Based Phenylalanine Flux Assay

The effect of the compounds disclosed herein to stabilize a mutant R408W protein was evaluated using a phenylalanine flux assay in A375-R408W cells. The AC₅₀ and maximum ¹³C₉¹⁵N-Tyrosine concentration obtained after protein stabilization are reported in Table 1 for Examples A through N.

Procedure: Cells expressing R408W PAH were made by transducing A375 cells with lentivirus encoding human PAH with the R408W mutation in pLVX-Puro, then selecting with puromycin until stable cell lines were generated. A375 R408W cells were seeded into 96 well plates in DMEM+10% FBS at a density of 40,000 cells/well one hour prior to compound addition. Compounds were resuspended in DMSO, and 2-fold serial dilutions were performed to generate a 10-point dose curve. Compounds were added to plated cells in a total volume of 100 μL, and a final DMSO concentration of 0.5%. Each compound was tested in duplicate. Following compound addition, cells were placed in a 5% CO₂, 37° C. tissue culture incubator for 24 hrs. After the incubation period, 20 μM sepiapterin and 800 μM ¹³C₉,¹⁵N-Phenylalanine were added. After 4 hours, cell media was removed. An aliquot of 10 μL of cell media was combined with 200 μL of extraction buffer (80% acetonitrile/20% H₂O) for each well. Determination of ¹³C-Tyrosine concentration was assessed by liquid chromatography mass spectrometry. Where a compound was tested multiple times, the average value of the tests is reported.

The corresponding R-enantiomer for one or more of the compounds described herein (i.e., compounds having the stereochemistry as defined in Formula Ib), were tested and found to be less active in the cell based phenylalanine flux assay. As such, the S-enantiomers of the compounds described herein (i.e., compounds having the stereochemistry as defined in Formula Ia) were pursued further and tested in a variety of other assays such as in those described below.

### Surface Plasmon Resonance (SPR) Binding Assay

Surface plasmon resonance (SPR) was used to measure the binding affinity (K_{D}) and binding kinetics (kₒₙ & k_{off}) of the compounds described herein with the R408W PAH protein in the presence of 1 mM of L-phenylalanine (Phe). The results for Examples A through N are shown in Table 1.

### Preparation of Human Mutant Avi-PAH R408W Protein

Cloning was designed to produce the fusion protein consisting of MBP-TEV cleavage site-Avi tag-PAH R408W (2-452). The TEV cleavage recognition sequence coded for was ENLYFQG (SEQ ID NO. 1), and the Avi tag sequence was Gly-Leu-Asn-Asp-Ile-Phe-Glu-Gln-Gln-Lys-Ile-Glu-Trp-His-Glu-Gly-Ser (SEQ ID NO. 2).

The baculovirus-directed expression system was used for recombinant protein expression. Baculovirus was generated by transfecting ~8×105 Sf9 cells cultured in a 6-well plate. The media was changed to Grace's insect medium at 27° C., and 1.5 g per well of bacmid was added to the cells using a Cellfectin II reagent. After transfection, the media was changed to SF-900 media. Passage (P)1 virus was harvested 72 hours after transfection. High-efficiency P2/P3 virus was generated by amplifying the P1/P2 virus by infecting an Sf9 cell suspension at 2×106 cells/mL for 72 hours with a multiplicity of infection of 0.01 to 0.5. Expression was conducted by infecting 2×106 Sf9 cells/mL (400 mL) in a 2-L shake flask with 40 mL of P3 virus. The culture was supplemented with 0.2 mM of ferrous ammonium sulfate. Flasks were maintained at 27° C. and 95 rpm for 48 hours, after which cells were pelleted at 500 g at 4° C. Cells were resuspended in PBS, 1 mM DTT, 5 mM EDTA, 20 mM Phenylalanine, pH7.4. Resuspended cells were lysed by passage through a microfluidizer at a pressure of 10,000 psi twice. Cell lysate was centrifuged at 40,000 g for 20 minutes at 4° C. and filtered through a 0.45 μm membrane.

All subsequent purification steps were performed at 4° C., unless otherwise stated. The supernatant was loaded onto an Amylose column (GE, XK26), pre-equilibrated in PBS, 1 mM DTT, 5 mM EDTA, 20 mM Phenylalanine, pH7.4. The column was then washed with 20 column volumes of the same buffer followed by elution in the wash buffer supplemented with 10 mM maltose.

Purified MBP-TEV-Avi-PAH R408W protein was cleaved by adding TEV protease at a ratio of 1:10 protease to total protein for 16 hours. The reaction mixture was then applied to a Superdex 200 gel filtration column that was pre-equilibrated in Buffer D containing 20 mM of 4-(2-hydroxyethyl)-1-piperazineethanesulfonic acid (HEPES) (purchased from Cytiva), 200 mM of NaCl, and 1 mM of L-Phe (purchased from Sigma-Aldrich, catalog #P2126), pH 7.0. Fractions containing the desired Avi-PAH protein were pooled and concentrated with a 50 kDa cutoff centrifugal filter to a concentration of approximately 2 mg/mL.

SPR Procedure: The SPR binding experiments were conducted on a Biacore 8K+ system at 15° C. using a human mutant Avi-tagged PAH R408W Protein (Avi-PAH R408W).

Avi-PAH R408W was captured on CM5 chips at 5,000 response units (RU). For experiments using the CM5 chip, neutravidin was first amine coupled to the CM5 surface at 25° C. Samples were prepared in 1× HBS-EP+ running buffer (purchased from Cytiva) comprising 10 mM of HEPES pH 7.4, 150 mM of NaCl, and 0.05% (v/v) P20 (purchased from Cytiva) supplemented with 3% (v/v) DMSO. Phe was included at a 1 μM concentration. SPR binding experiments were performed using a multi-cycle kinetics method. Multi-cycle kinetic experiments were run in a 3-fold, 8-concentration series with a top concentration of 5 M. Association and dissociation times were 60 seconds and 300 seconds, respectively.

Software from the instrument manufacturer (Cytiva) was used for data analysis. For data collected in multi-cycle kinetics mode on the Biacore 8k+ instrument, Biacore Insight Evaluation Software version 3.0 was used.

The kinetically derived K_{D} (equilibrium dissociation constant) is calculated as the ratio of the experimentally measured dissociation rate constant (k_{off}) to the association rate constant (kₒₙ): K_{D}=k_{off}/kₒₙ.

TABLE 1
Cellular Phenylalanine Flux Assay and Surface Plasmon Resonance
Binding Assay Results

| | | Phenylalanine | | | |
|---|---|---|---|---|---|
| | | Flux Assay | | | |
| | | Maximum | | | |
| | | ¹³C₉¹⁵N- | | | |
| | Phenylalanine | Tyrosine | | SPR | |
| | Flux Assay | Concentration | SPR | Kₒₙ | SPR |
| Compound | AC₅₀ (μM) | (μM) | K_{D} (μM) | (M⁻¹.s⁻¹) | K_{off} (s⁻¹) |
| 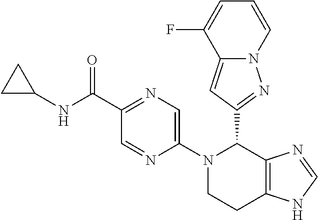 [SMILES: O=C(NC1CC1)c1cnc(N2CCc3[nH]cnc3[C@H]2c2cc3c(F)cccn3n2)cn1] | 0.148 | 23.4 | 0.487 | 5.84E4 | 0.028 |
| Example A | | | | | |
| 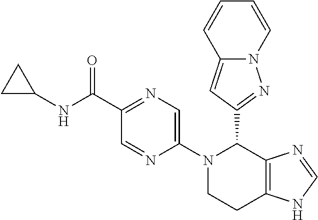 [SMILES: O=C(NC1CC1)c1cnc(N2CCc3[nH]cnc3[C@H]2c2cc3ccccn3n2)cn1] | 0.348 | 25.3 | 1.15 | 2.81E4 | 0.032 |
| Example B | | | | | |
| 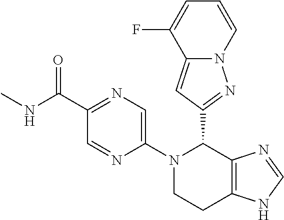 [SMILES: CNC(=O)c1cnc(N2CCc3[nH]cnc3[C@H]2c2cc3c(F)cccn3n2)cn1] | 0.444 | 27.7 | 1.15 | 6.15E4 | 0.071 |
| Example C | | | | | |
| 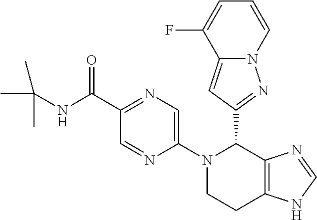 [SMILES: CC(C)(C)NC(=O)c1cnc(N2CCc3[nH]cnc3[C@H]2c2cc3c(F)cccn3n2)cn1] | 0.088 | 20.3 | 1.272 | 1.76E4 | 0.022 |
| Example D | | | | | |
| 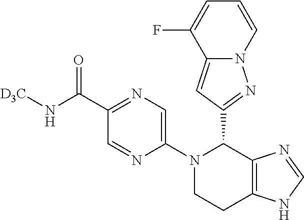 [SMILES: [2H]C([2H])([2H])NC(=O)c1cnc(N2CCc3[nH]cnc3[C@H]2c2cc3c(F)cccn3n2)cn1] | 0.518 | 29.5 | 1.472 | 4.73E4 | 0.070 |
| Example E | | | | | |
| 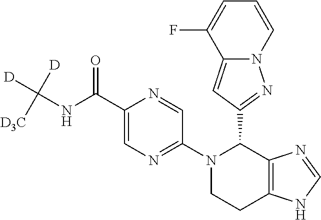 [SMILES: [2H]C([2H])([2H])C([2H])([2H])NC(=O)c1cnc(N2CCc3[nH]cnc3[C@H]2c2cc3c(F)cccn3n2)cn1] | 0.207 | 25.0 | 0.876 | 6.55E4 | 0.057 |
| Example F | | | | | |
| 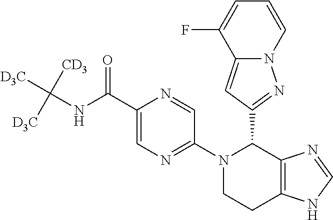 [SMILES: [2H]C([2H])([2H])C(NC(=O)c1cnc(N2CCc3[nH]cnc3[C@H]2c2cc3c(F)cccn3n2)cn1)(C([2H])([2H])[2H])C([2H])([2H])[2H]] | 0.059 | 21.0 | 0.914 | 6.42E4 | 0.059 |
| Example G | | | | | |
| 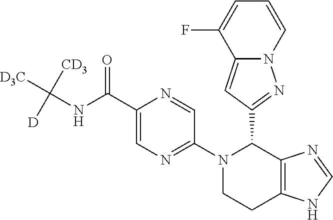 [SMILES: [2H]C([2H])([2H])C([2H])(NC(=O)c1cnc(N2CCc3[nH]cnc3[C@H]2c2cc3c(F)cccn3n2)cn1)C([2H])([2H])[2H]] | 0.120 | 20.0 | 1.021 | 2.37E4 | 0.024 |
| Example H | | | | | |
| 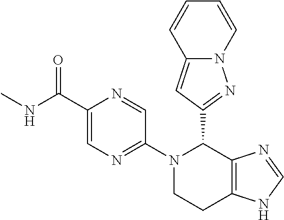 [SMILES: CNC(=O)c1cnc(N2CCc3[nH]cnc3[C@H]2c2cc3ccccn3n2)cn1] | 1.52 | 27.0 | 1.81 | 4.57E4 | 0.083 |
| Example I | | | | | |
| 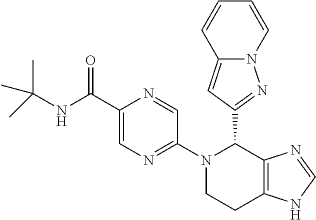 [SMILES: CC(C)(C)NC(=O)c1cnc(N2CCc3[nH]cnc3[C@H]2c2cc3ccccn3n2)cn1] | 0.102 | 19.0 | 1.95 | 2.00E4 | 0.039 |
| Example J | | | | | |
| 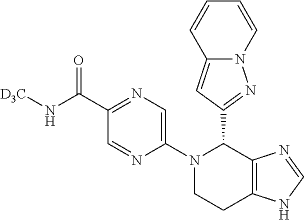 [SMILES: [2H]C([2H])([2H])NC(=O)c1cnc(N2CCc3[nH]cnc3[C@H]2c2cc3ccccn3n2)cn1] | 1.182 | 27.0 | 1.40 | 5.87E4 | 0.082 |
| Example K | | | | | |
| 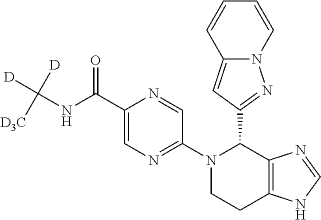 [SMILES: [2H]C([2H])([2H])C([2H])([2H])NC(=O)c1cnc(N2CCc3[nH]cnc3[C@H]2c2cc3ccccn3n2)cn1] | 0.688 | 26.0 | 2.03 | 4.26E4 | 0.086 |
| Example L | | | | | |
| 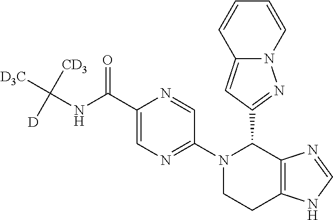 [SMILES: [2H]C([2H])([2H])C([2H])(NC(=O)c1cnc(N2CCc3[nH]cnc3[C@H]2c2cc3ccccn3n2)cn1)C([2H])([2H])[2H]] | 0.154 | 23.0 | 1.85 | 4.41E4 | 0.082 |
| Example M | | | | | |
| 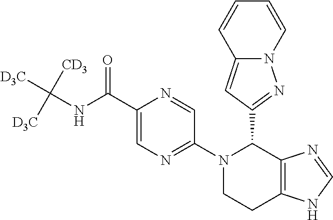 [SMILES: [2H]C([2H])([2H])C(NC(=O)c1cnc(N2CCc3[nH]cnc3[C@H]2c2cc3ccccn3n2)cn1)(C([2H])([2H])[2H])C([2H])([2H])[2H]] | 0.124 | 21.0 | 1.75 | 2.09E4 | 0.037 |
| Example N | | | | | |

### In Vitro Metabolic Stability in Human Liver Microsomes

The in vitro metabolic stability of the compounds described herein were assessed in human liver microsomes. The hepatic extraction ratio (Eₕ) for Examples A through N are shown in Table 2. In Table 2, where the test compound was greater than 80% remaining at maximal incubation time (45 min; T_{1/2}>135 min), the hepatic extraction ratio cut-off value of <0.29 was reported, and where the test compound was less than 80% remaining at maximal incubation time, the calculated hepatic extraction ratio values were reported.

Procedure: Pooled human liver microsomes (mixed gender, Catalog #H0610) were purchased from Sekisui XenoTech (Kansas City, KS, USA). Nicotinamide adenine dinucleotide phosphate (NADPH) tetrasodium salt (Catalog #HY-1F0003) was purchased from MedChem Express (Monmouth Junction, NJ 08852, USA). Ketanserin (Catalog #S006-50 mg) was purchased from Sigma Aldrich (St Louis, MO, USA).

Human liver microsomes (final concentration of 0.5 mg/mL in 0.1 M potassium phosphate buffer, pH 7.4) were incubated with 1 μM of either the test compound or reference compound ketanserin for 0, 5, 15, 30, and 45 minutes in duplicate. Reactions were initiated by the addition of NADPH (final concentration of 2 mM) to all wells except the 0-minute time point. The final concentration of organic solvents in the reactions were <0.25% v/v. At the end of incubation, reactions were stopped by the addition of acetonitrile containing imipramine (1 μM) as an internal standard. For the 0-minute time point, acetonitrile containing imipramine was added to the well followed by NADPH. The assay plates were shaken and then centrifuged at 3,220 g for 10 minutes to pellet the precipitated protein. The supernatants were diluted with Mill-Q water before being analyzed by liquid chromatography with tandem mass spectrometry.

To calculate the results, the natural log of the percent remaining was plotted against time, and the slope of the line calculated. Percent remaining is the ratio of the compound peak area to the internal standard peak area at each time point, expressed as a percentage of the value at 0 minutes. The slope is used to determine the rate constant of elimination (kₑₗ) as the negative of the slope, and the half-life (ti/2) is then calculated using the formula 0.693/kₑₗ.

The scaled in vitro intrinsic clearance (CLᵢₙₜ), in L/hr/kg was calculated using Equation 1.

$${CL}_{int} = \frac{\begin{matrix}{k}_{el} \times ( \frac{mL incubation volume}{mg protein per incubation} ) \times \\ MPPGL ( mg / g ) \times liver weight ( g / kg ) \times 60\end{matrix}}{1000} \qquad Equation 1$$

where the value of liver weight per kilogram of body weight used was 25.7 g/kg and the value of milligrams protein per gram of liver (MPPGL) used was 32.0 mg/g.

Hepatic clearance (CLₕₑₚ), in L/hr/kg, was then calculated using Equation 2.

$${CL}_{hep} = \frac{{CL}_{int} \times QH}{{CL}_{int} + QH} \qquad Equation 2$$

where the hepatic blood flow (QH) value used was 1.2 L/hr/kg.

The hepatic extraction ratio (Eₕ) was calculated by Equation 3.

$${E}_{h} = \frac{{CL}_{hep}}{QH} \qquad Equation 3$$

### In Vitro Evaluation of Cellular Permeability Across a Caco-2 Cell Monolayer

The cellular permeability of the compounds described herein was determined using Caco-2 cell lines. The permeability coefficient (Pₐₚₚ) from apical-to-basolateral (A-to-B) and basolateral-to-apical (B-to-A) directions and the efflux ratio for Examples A through N are shown in Table 2.

Procedure: Caco-2 cells were purchased from American Tissue Culture Collection (Rockville, MD, USA; catalog #HTB-37). Hanks Balanced Salt Solution (HBSS) was purchased from Life Technologies (Carlsbad, CA, USA, catalog #14025-092). Lucifer yellow (LY) was purchased from Sigma-Aldrich (St Louis, MO, USA). Erythromycin, metoprolol, and atenolol were purchased from NICPBP (Shanghai, P.R. China). Millicell-24 cell culture insert plates were purchased from Millipore (Burlington, MA, USA; catalog #PSHT010R5). A 10-μM stock solution of the test compound was prepared in DMSO, and stored at −20° C.

Caco-2 cells were maintained in Modified Eagle's medium, containing 10% heat-inactivated fetal bovine serum and 1% nonessential amino acids. Cells were seeded on polycarbonate filter inserts and transepithelial electrical resistance (TEER) was used to determine when the monolayer integrity was sufficient to begin the experiment (>250 Ω×cm²).

Immediately before the transport experiment, the cell monolayers were washed with HBSS, and TEER values were measured. Stock solutions of the test compound and the reference compound cassette of erythromycin/metoprolol/atenolol (10 mM) were prepared in DMSO. Working solutions at 10 μM for the test compound and the reference compounds were prepared by diluting the above stock solutions in an assay buffer (HBSS with 25-mM HEPES, pH 7.4). Transport of the compounds across the monolayers was conducted at 37° C. for 90 minutes in duplicate in both the apical-to-basolateral (A-to-B) and basolateral-to-apical (B-to-A) directions. Zero-minute time point samples were taken prior to the addition of the donor wells to the receiver wells. The final DMSO content in the transport system was 0.4%. Lucifer yellow (5 μM) was used to confirm monolayer integrity in the experiment. At the end of incubation, 60 μL of the receiver samples as well as 10-fold diluted donor samples were mixed with 60 μL of acetonitrile and analyzed by LC-MS/MS.

Apparent permeability and efflux ratio were calculated for the reference and test compounds as described below.

Transepithelial electrical resistance was calculated using Equation 4.

$$TEER = {Resistance}_{sample} - {Resistance}_{blank} \times Membrane Area \qquad Equation 4$$

Lucifer yellow permeability was calculated using Equation 5.

$${P}_{app} = \frac{{V}_{A}}{Area \times Time} \times \frac{{RFU}_{t = 90 , receiver} - {RFU}_{blank}}{( {RFU}_{t = 0 , donor} - {RFU}_{blank} ) \times dilution factor} \qquad Equation 5$$

where V_{A} is the volume in the receiver well (0.8 mL for A-to-B and 0.4 mL for B-to-A), area is the surface area of the membrane (0.7 cm² for Millicell-24 cell culture plates), time is the total transport time in seconds, and RFU is relative fluorescent units.

Drug permeability was calculated using Equation 6.

$${P}_{app} = \frac{{V}_{A}}{Area \times Time} \times \frac{{[ Drug ]}_{t = 90 , receiver} - {[ Drug ]}_{blank}}{( {[ Drug ]}_{t = 0 , donor} - {[ Drug ]}_{blank} ) \times Dilution Factor} \qquad Equation 6$$

where V_{A} is the volume in the receiver well (0.8 mL for A-to-B and 0.4 mL for B-to-A), area is the surface area of the membrane (0.7 cm² for Millicell-24 cell culture plates), and time is the total transport time in seconds.

Efflux ratio was calculated using Equation 7.

$$Efflux Ratio = \frac{{P}_{app} ( B - A )}{{P}_{app} ( A - B )} \qquad Equation 7$$

### Oral Bioavailability in Rats

The oral bioavailability for some of the compounds described herein were assessed in rats, and the results (mean % F) reported in Table 2.

Male Sprague Dawley rats (n=3/group) weighing 200-240 g and aged 6-8 weeks were obtained from Jihui Laboratory Animal Co., LTD. The formulation vehicles used for IV dose was 10% v/v NMP, 10% v/v Solutol HS 15, and 80% v/v saline and for PO dose was 20% w/v Captisol and 20% w/v Solutol HS 15 in 50 mM sodium citrate buffer (pH 4.0). The PO dose group animals were fasted overnight and fed after 4 hrs post dose, while IV-group animals were not. The IV dose (3 mg/kg, 2 mL/kg) was administered via foot dorsal vein bolus, and PO doses (10 mg/kg, 10 mL/kg) via gavage. Blood (~150 μL) was collected via tail vein at 0 (pre-dose), 0.083, 0.25, 0.5, 1, 2, 4, 8 and 24 hr post dose in tubes containing K₂EDTA as anticoagulant.

Blood samples were put on ice immediately upon collection and centrifuged at 2000 g for 5 min to obtain plasma samples within 15 minutes of sample collection. Plasma was stored at −70° C. for LC-MS/MS analysis. A qualified LC-MS/MS method with protein precipitation as sample preparation was used for quantification, employing an SCIEX API6500+ mass spectrometer (SCIEX, Framingham Massachusetts [MA], US) with a Waters Acquity UPLC® system (Waters, Milford, MA, US), using reversed-phase gradient separation and positive ion spray ionization. Pharmacokinetic analysis of the individual subject plasma concentration data was performed using Phoenix WinNonlin® 8.2. The area under the concentration time curve (AUC) was obtained by noncompartmental analysis using the linear trapezoidal rule. The concentrations that were less than the LLOQ were designated as BLQ. The oral bioavailability was calculated as:

$$F = \frac{{AUC}_{INF} PO \times {Dose}_{IV}}{mean {AUC}_{INF} IV \times {Dose}_{PO}} .$$

TABLE 2
In vitro metabolic stability in human liver microsomes, in vitro cellular
permeability, and oral bioavailability assay results

| | | Cell | Cell | | |
|---|---|---|---|---|---|
| | Hepatic | Permeability | Permeability | Cell | Rat Oral |
| | Extraction | Pₐₚₚ A-to-B | Pₐₚₚ B-to-A | Permeability | Bioavailability |
| Compound | Ratio (Eₕ) | x10⁻⁶ cm/s | x10⁻⁶ cm/s | Efflux Ratio | % F |
| 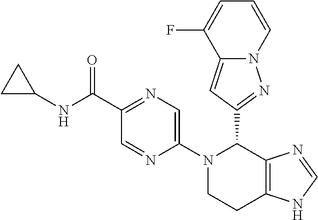 [SMILES: O=C(NC1CC1)c1cnc(N2CCc3[nH]cnc3[C@H]2c2cc3c(F)cccn3n2)cn1] | <0.29 | 5.28 | 57.1 | 10.8 | 54.9 |
| Example A | | | | | |
| 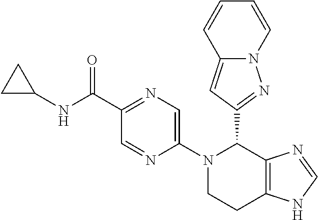 [SMILES: O=C(NC1CC1)c1cnc(N2CCc3[nH]cnc3[C@H]2c2cc3ccccn3n2)cn1] | <0.29 | 3.13 | 40.7 | 13.0 | 30.0 |
| Example B | | | | | |
| 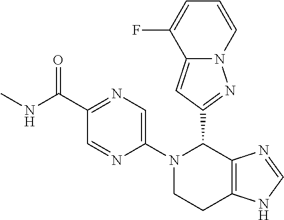 [SMILES: CNC(=O)c1cnc(N2CCc3[nH]cnc3[C@H]2c2cc3c(F)cccn3n2)cn1] | <0.29 | 3.03 | 38.2 | 12.6 | 59.1 |
| Example C | | | | | |
| 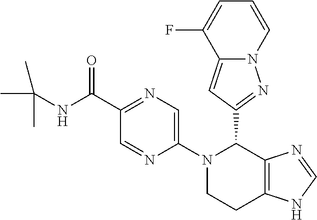 [SMILES: CC(C)(C)NC(=O)c1cnc(N2CCc3[nH]cnc3[C@H]2c2cc3c(F)cccn3n2)cn1] | <0.29 | 20.6 | 48.9 | 2.37 | 87.7 |
| Example D | | | | | |
| 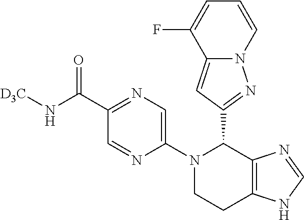 [SMILES: [2H]C([2H])([2H])NC(=O)c1cnc(N2CCc3[nH]cnc3[C@H]2c2cc3c(F)cccn3n2)cn1] | <0.29 | 4.70 | 33.2 | 7.07 | 67.6 |
| Example E | | | | | |
| 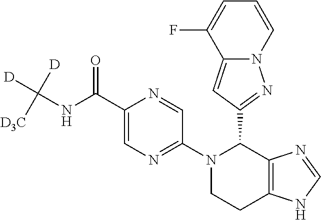 [SMILES: [2H]C([2H])([2H])C([2H])([2H])NC(=O)c1cnc(N2CCc3[nH]cnc3[C@H]2c2cc3c(F)cccn3n2)cn1] | <0.29 | 6.87 | 48.9 | 7.11 | NT |
| Example F | | | | | |
| 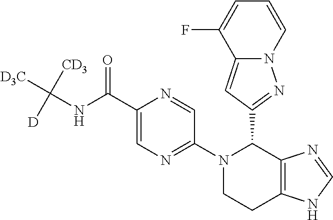 [SMILES: [2H]C([2H])([2H])C([2H])(NC(=O)c1cnc(N2CCc3[nH]cnc3[C@H]2c2cc3c(F)cccn3n2)cn1)C([2H])([2H])[2H]] | <0.29 | 9.17 | 51.2 | 5.59 | NT |
| Example G | | | | | |
| 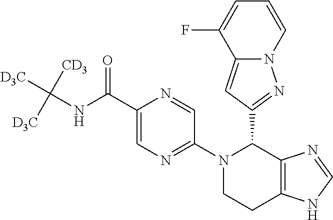 [SMILES: [2H]C([2H])([2H])C(NC(=O)c1cnc(N2CCc3[nH]cnc3[C@H]2c2cc3c(F)cccn3n2)cn1)(C([2H])([2H])[2H])C([2H])([2H])[2H]] | 0.37 | 13.2 | 42.5 | 3.21 | NT |
| Example H | | | | | |
| 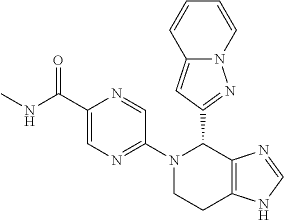 [SMILES: CNC(=O)c1cnc(N2CCc3[nH]cnc3[C@H]2c2cc3ccccn3n2)cn1] | <0.29 | 2.66 | 33.3 | 12.5 | NT |
| Example I | | | | | |
| 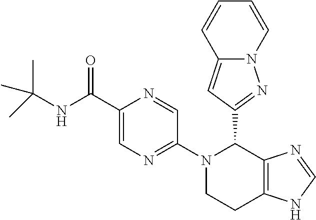 [SMILES: CC(C)(C)NC(=O)c1cnc(N2CCc3[nH]cnc3[C@H]2c2cc3ccccn3n2)cn1] | 0.30 | 10.2 | 55.6 | 5.47 | NT |
| Example J | | | | | |
| 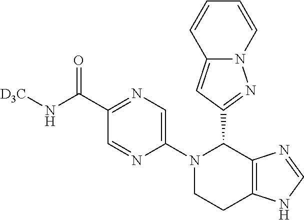 [SMILES: [2H]C([2H])([2H])NC(=O)c1cnc(N2CCc3[nH]cnc3[C@H]2c2cc3ccccn3n2)cn1] | <0.29 | 2.07 | 26.8 | 13.0 | NT |
| Example K | | | | | |
| 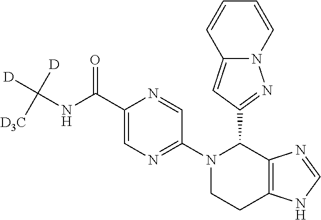 [SMILES: [2H]C([2H])([2H])C([2H])([2H])NC(=O)c1cnc(N2CCc3[nH]cnc3[C@H]2c2cc3ccccn3n2)cn1] | <0.29 | 8.11 | 23.8 | 2.93 | NT |
| Example L | | | | | |
| 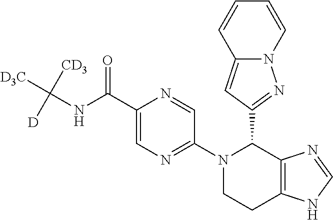 [SMILES: [2H]C([2H])([2H])C([2H])(NC(=O)c1cnc(N2CCc3[nH]cnc3[C@H]2c2cc3ccccn3n2)cn1)C([2H])([2H])[2H]] | <0.29 | 10.1 | 30.0 | 2.97 | NT |
| Example M | | | | | |
| 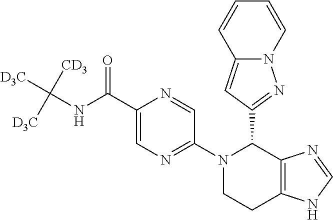 [SMILES: [2H]C([2H])([2H])C(NC(=O)c1cnc(N2CCc3[nH]cnc3[C@H]2c2cc3ccccn3n2)cn1)(C([2H])([2H])[2H])C([2H])([2H])[2H]] | <0.29 | 20.7 | 36.4 | 1.76 | NT |
| Example N | | | | | |

### Aromatase Inhibition Assay

The inhibition potential of the compounds described herein on the aromatase (CYP19A1) enzyme was determined using cDNA-expressed human CYP19A1 enzyme (Cat 456260, Corning) with androstenedione as the substrate. The inhibition potential for Examples A through N are reported as an IC₅₀ value in Table 3 below. An IC₅₀ value of >30 M is reported in Table 3 if the inhibition curve fitting result was greater than 30 μM (the maximum test concentration in this assay) or if the maximum inhibition at 30 μM was lower than 50%.

An aliquot (1 μL) of multiple concentrations (4.12, 12.35, 37.03, 111.11, 333.33, 1000 and 3000 μM) of test compounds or positive control ketoconazole, prepared in DMSO, were incubated 5 min at 37° C. in buffer (100 mM PBS pH 7.4, containing 3.3 mM MgCl₂) with 0.5 pmol/mL cDNA-expressed human CYP19A1 enzyme and substrate androstenedione (150 nM). DMSO was used as the vehicle control. The final concentrations of test compounds in the incubation system were 0.041, 0.12, 0.37, 1.11, 3.33, 10 and 30 PM. The reaction was initiated by adding NADPH (2 mM final concentration), pre-warmed at 37° C. in water bath for 5 minutes. The reaction was carried out in a 37° C. water bath for 15 minutes. All incubations were performed in duplicate. At the end of the incubation, the reaction was quenched by 600 μL of cold quench solution (85:15 (v:v) hexane/ethyl acetate containing 500 nM cortisone as an internal standard). Samples were vortexed thoroughly and centrifuged at 3,220 g for 10 min at 4° C. The supernatant (400 μL) was evaporated to dryness under a constant stream of nitrogen at room temperature. The samples were reconstituted with 200 μL of 70:30 (v:v) methanol/water and were vortexed thoroughly. The rate of formation of isoform-specific metabolite derived from androstenedione was measured by UPLC-MS/MS. A decrease in the formation of the metabolite in peak area ratio to vehicle control was used to calculate an IC₅₀ value (test compound concentration which produces 50% inhibition) by using Prism 8.0.2 software (Graphpad).

In Vitro Evaluation of hERG Inhibition

The inhibition potential of the compounds described herein on the hERG potassium ion channel was measured using a hERG patch clamp assay. The inhibition potential for Examples A through N are reported as IC₅₀ values in Table 3. In Table 3, an IC₅₀>100 μM was reported if the inhibition curve fitting result was greater than 100 μM (the maximum test concentration in this assay) or if the maximum inhibition at 100 μM was lower than 50%. The IC₅₀ values reported in Table 3 for Examples A, C, and E were obtained using the manual patch clamp assay, and the remaining values were obtained using an automated QPatch assay. The IC₅₀ reported for Example C in Table 3 is an extrapolated IC₅₀ due to a procedural issue wherein the hERG inhibition rate of the test compound solution at 100 μM could not be obtained.

Procedure for Automated QPatch Assay: A CHO cell line stably transfected with hERG cDNA and expressing hERG channels of P10 was used for the study. Cells were maintained in petri dishes or flasks at 37° C. in a humidified incubator with 5% CO₂ and cultured in medium (from Invitrogen) containing: Ham's F12, 10% (v/v) heat inactivated FBS, 100 μg/ml Hygromycin B, and 100 μg/ml Geneticin.

Cells were allowed to grow and reach a confluence rate at about 80-90% in the above condition. Before the experiment, the cells were treated with Detachin (from Genlantis) for 3-5 minutes at 37° C. followed by gentle pipette trituration 15-20 times at room temperature with culture media, then the cells were re-suspended in serum-free culture medium containing CHO-S-SFM II medium (from Invitrogen) buffered with HEPES (25 mM). The cells were used in the QPatch study if they met the following criteria: under microscopy examination, the majority of cells in suspension were single and isolated; their viability was greater than 95%, with only a few debris and cell clumps (which may clog the holes in QPlate during whole-cell clamp recording); cell density was ranged within 3-8×10⁶ cells/ml in the final suspension before applying to the QPatch stir chamber. Cells in such condition can be used for recording only for four hours after harvesting.

For the electrophysiological recordings the following solutions were used.

Composition of internal and external
solutions used in hERG studies

| Reagent | External Solution (mM) | Internal Solution (mM) |
|---|---|---|
| CaCl₂ | 2 | 5.37 |
| MgCl₂ | 1 | 1.75 |
| KCl | 4 | 120 |
| NaCl | 145 | — |
| Glucose | 10 | — |
| HEPES | 10 | 10 |
| EGTA | — | 5 |
| Na-ATP | — | 4 |
| pH & Osmolarity | 7.4 (adjusted with KOH), | 7.25 (adjusted with KOH), |
| | osmolarity approx. 305 | osmolarity approx. 295 |
| | mOsm | mOsm |

Whole-cell recordings were performed using automated QPatch (Sophion Biosciences, Denmark). The cells were voltage clamped at a holding potential of −80 mV. The hERG current was activated by depolarizing at +20 mV for 5 sec, after which the current was taken back to −50 mV for 2.5 sec to remove the inactivation and observe the outward tail current. The maximum amount of tail current was used to determine hERG current amplitude.

Six doses (100, 30, 10, 3, 1, and 0.3 μM) of the test compound were chosen for evaluation. Before testing, the test compounds were made into 100 μM DMSO stock solution, and then serial-diluted in external solution to the desired concentrations. Sonication and vortex (5-10 minutes) were routinely applied to the solutions to achieve the complete dissolution of the test compound prior to testing. Final DMSO concentration of compounds was 0.1%, except for the 100 μM dose which was about 1%. Doses for IC₅₀ evaluation of positive control cisapride were 3, 1, 0.3, 0.1, 0.03, and 0.01 μM.

After achieving break-in (whole-cell) configuration, the cells were recorded for 120 sec to assess current stability. The voltage protocol described above was then applied to the cells every 15 sec throughout the whole procedure. Only stable cells with recording parameters above threshold were allowed to enter the drug application procedure. All experiments were conducted at room temperature (about 25° C.). External solution containing 0.1% DMSO (vehicle) was applied to the cells to establish the baseline. After allowing the current to stabilize for 3 minutes, the test compound was applied.

The test compound solution was added, and the cells were kept in the test solution until the test compound's effect reached a steady state or for a maximum of 4 min. For dose response assay, compound was applied to the cells accumulatively from low to high concentrations. Washout with external solution was performed after compound testing. Positive control cisapride was used in the experiments to test the same batch of cells used for test compounds to ensure the normal response and the good quality of the cells. Data were analyzed using Assay Software provided by Sophion (assay software V5.0), Microsoft Excel and Graphpad Prism 5.0.

In addition to the automated QPatch assay, the hERG inhibition potential can be measured using a manual patch clamp assay.

Procedure for manual whole cell patch and recording: HEKA EPC 10 USB patch clamp amplifier (from HEKA Elektronik, Germany) was used in the whole cell recording. A cover slip with plenty of single CHO hERG cells on the surface was removed and placed into a continuously perfused (approximately 1 ml/minute) recording chamber mounted on an inverted microscope. HERG channel currents were recorded from single cells using standard whole cell recording techniques. The cells were voltage clamped at a holding potential of −80 mV. The hERG current was activated by depolarizing at +20 mV for 5 sec, after which the current was taken back to −50 mV for 5 sec to remove the inactivation and observe the deactivating tail current. The K⁺ tail current through hERG channels observed during this step was allowed to stabilize under continuous bath perfusion. Cells were then superfused with test compound until steady state block was achieved. Steady state was considered reached when three consecutive super-imposable current records were collected. At this point, cells were once again superfused with extracellular solution until the current amplitude returned to values close to those measured before application of the test compound. One or more concentrations (100, 30, 10, 3, 1, and 0.3 μM) of test compounds were tested on each cell with washout in between each compound application. Cisapride was used in the experiments to ensure the normal response and good quality of the hERG cells.

TABLE 3
In Vitro Aromatase and hERG Inhibition Assay Results

| | Aromatase Inhibition | hERG Inhibition |
|---|---|---|
| Compound | IC₅₀ (μM) | IC₅₀ (μM) |
| 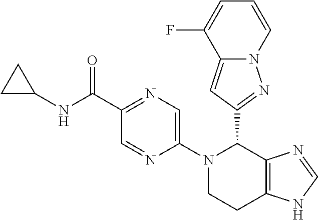 [SMILES: O=C(NC1CC1)c1cnc(N2CCc3[nH]cnc3[C@H]2c2cc3c(F)cccn3n2)cn1] | >30 | 35.0 |
| Example A | | |
| 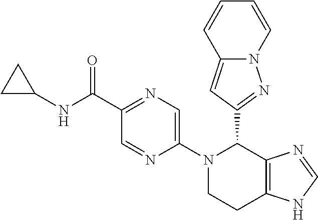 [SMILES: O=C(NC1CC1)c1cnc(N2CCc3[nH]cnc3[C@H]2c2cc3ccccn3n2)cn1] | 26.3 | 89.8 |
| Example B | | |
| 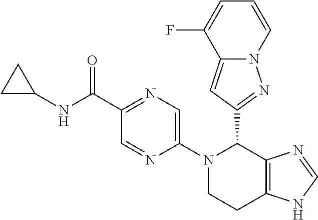 [SMILES: O=C(NC1CC1)c1cnc(N2CCc3[nH]cnc3[C@H]2c2cc3c(F)cccn3n2)cn1] | >30 | 61.0 |
| Example C | | |
| 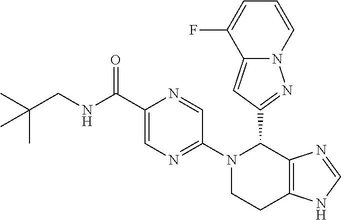 [SMILES: CC(C)(C)CNC(=O)c1cnc(N2CCc3[nH]cnc3[C@H]2c2cc3c(F)cccn3n2)cn1] | 12.2 | >100 |
| Example D | | |
| 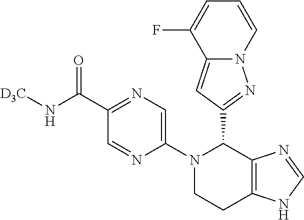 [SMILES: [2H]C([2H])([2H])NC(=O)c1cnc(N2CCc3[nH]cnc3[C@H]2c2cc3c(F)cccn3n2)cn1] | >30 | 79.0 |
| Example E | | |
| 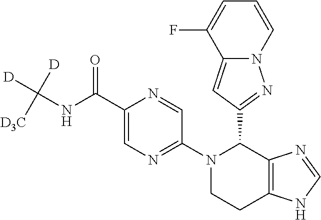 [SMILES: [2H]C([2H])([2H])C([2H])([2H])NC(=O)c1cnc(N2CCc3[nH]cnc3[C@H]2c2cc3c(F)cccn3n2)cn1] | >30 | 26.7 |
| Example F | | |
| 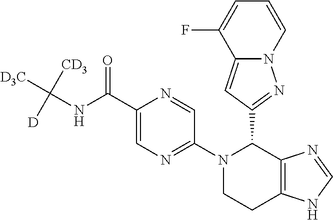 [SMILES: [2H]C([2H])([2H])C([2H])(NC(=O)c1cnc(N2CCc3[nH]cnc3[C@H]2c2cc3c(F)cccn3n2)cn1)C([2H])([2H])[2H]] | 20.3 | 33.8 |
| Example G | | |
| 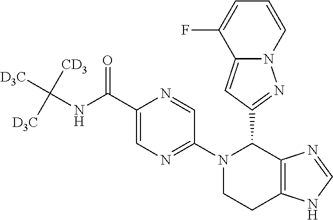 [SMILES: [2H]C([2H])([2H])C(NC(=O)c1cnc(N2CCc3[nH]cnc3[C@H]2c2cc3c(F)cccn3n2)cn1)(C([2H])([2H])[2H])C([2H])([2H])[2H]] | 8.1 | 29.5 |
| Example H | | |
| 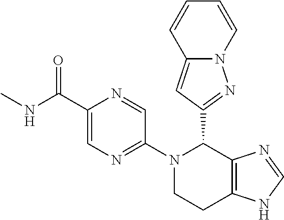 [SMILES: CNC(=O)c1cnc(N2CCc3[nH]cnc3[C@H]2c2cc3ccccn3n2)cn1] | >30 | 93.4 |
| Example I | | |
| 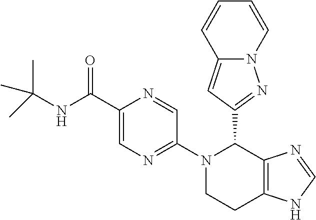 [SMILES: CC(C)(C)NC(=O)c1cnc(N2CCc3[nH]cnc3[C@H]2c2cc3ccccn3n2)cn1] | 7.5 | 27.8 |
| Example J | | |
| 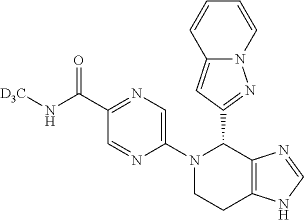 [SMILES: [2H]C([2H])([2H])NC(=O)c1cnc(N2CCc3[nH]cnc3[C@H]2c2cc3ccccn3n2)cn1] | >30 | 78.4 |
| Example K | | |
| 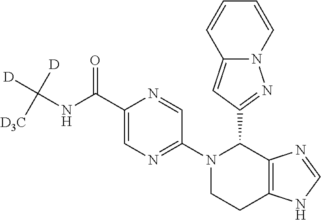 [SMILES: [2H]C([2H])([2H])C([2H])([2H])NC(=O)c1cnc(N2CCc3[nH]cnc3[C@H]2c2cc3ccccn3n2)cn1] | 28.8 | 29.4 |
| Example L | | |
| 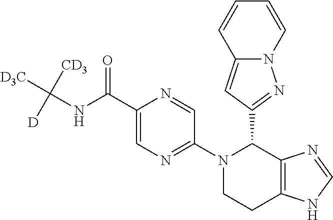 [SMILES: [2H]C([2H])([2H])C([2H])(NC(=O)c1cnc(N2CCc3[nH]cnc3[C@H]2c2cc3ccccn3n2)cn1)C([2H])([2H])[2H]] | 26.0 | 40.3 |
| Example M | | |
| 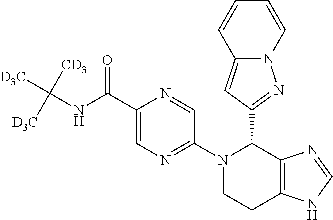 [SMILES: [2H]C([2H])([2H])C(NC(=O)c1cnc(N2CCc3[nH]cnc3[C@H]2c2cc3ccccn3n2)cn1)(C([2H])([2H])[2H])C([2H])([2H])[2H]] | 8.4 | 21.4 |
| Example N | | |

### In Vitro Evaluation of the Inhibition Potential of Test Compounds of Cytochrome P450 (CYP) Enzymes in Human Liver Microsomes (HLMs)

The compounds described herein were tested for their ability to inhibit Cytochrome P450 (CYP) isoforms CYP1A2, CYP2C19, CYP2C9, CYP21D6, and CYP3A4. The inhibition potential for Examples A through N are reported as IC₅₀ values in Table 4. In Table 4, the IC₅₀ value is reported as >100 μM if the inhibition curve fitting result was greater than 100 M (the maximum test concentration in this assay) or if the maximum inhibition at 100 μM was lower than 50%.

Procedure: The reversible inhibition potential for test compounds to inhibit the hepatic cytochrome P450 enzymes was evaluated using a specific probe substrate for each enzyme (CYP1A2: Phenacetin at 30 μM; CYP2C9: Diclofenac at 10 PM; CYP2C19: S-Mephenytoin at 35 μM; CYP2D6: Bufuralol at 10 μM; and CYP3A4: Midazolam at 5 μM) in discrete incubations with pooled HLMs (mixed gender, Catalog #452117), purchased from Corning. The positive control inhibitors used in the studies were a-naphthoflavone (CYP1A2), sulfaphenazole (CYP2C9), omeprazole (CYP2C19), ketoconazole (CYP3A4), and quinidine (CYP2D6). Serial dilutions of test compounds (400×) and reference inhibitors are prepared in a 96-well plate using a DMSO:ACN mixture. A NADPH solution (8 mM) was prepared by dissolving 66.7 mg of NADPH in 10 mL of 0.1 M potassium phosphate buffer (pH 7.4). The 4× substrate solution (2 mL per isoform) was prepared in a phosphate buffer containing HLM and kept on ice. Additionally, a 0.2 mg/mL HLM solution was prepared in phosphate buffer and kept on ice. To the assay wells, 400 μL of 0.2 mg/mL HLM was added, followed by 2 μL of 400× serially diluted test compound in designated wells. In a separate well, a 200 μL of 0.2 mg/mL HLM and 1 μL of serially diluted reference inhibitor solution was added. In a 96-well assay plate (in duplicate, on ice), 30 μL of 2× test/reference compound in 0.2 mg/mL HLM solution was mixed with 15 μL of 4× substrate solution, and the assay plate pre-incubated at 37° C. for 5 minutes. Then, the reaction was initiated by addition of 15 μL of NADPH (8 mM) solution preincubated at 37° C. for 5 minutes. The incubation was carried out at 37° C. for different time intervals: 5 minutes for CYP3A4, 10 minutes for CYP1A2, CYP2C9, and CYP2D6, and 45 minutes for CYP2C19. The reaction was stopped by adding 120 μL of ACN containing an internal standard (CYP1A2: Acetaminophen-D₄, CYP2C19: Hydroxymephenytoin-D₃, CYP3A4: 1′-hydroxymidazolam-D₄, CYP2C9: 4′-hydroxydiclofenac-[¹³C6], and CYP2D6: 1-hydroxybufuralol-maleate-[D₉]), then shaken for 10 minutes at 600 rpm (IKA, MTS 2/4) and centrifuged at 5594×g for 15 minutes (Thermo Multifuge×3R). Finally, 50 μL of the supernatant from each well was transferred into a 96-well sample plate containing 50 μL of ultrapure water for LC/MS analysis. The metabolites formed for each probe substrate (CYP1A2: acetaminophen, CYP2C9: 4-hydroxdiclofenac, CYP2C19: hydroxymephenytoin, CYP2D6: 1-hydroxybufuralol, and CYP3A4: hydroxymidazolam) were determined using an LC-MS/MS. A decrease in the formation of the metabolite in peak area ratio to vehicle control was used to calculate the IC₅₀ value (test compound concentration which produces 5000 inhibition) by using Prism 5.0 software (Graphpad).

TABLE 4
CYP Inhibition Assay Results

| | Cyp 1A2 | Cyp 2C19 | Cyp 2C9 | Cyp 2D6 | Cyp 3A4 |
|---|---|---|---|---|---|
| | Inhibition | Inhibition | Inhibition | Inhibition | Inhibition |
| Compound | IC₅₀ (μM) | IC₅₀ (μM) | IC₅₀ (μM) | IC₅₀ (μM) | IC₅₀ (μM) |
| 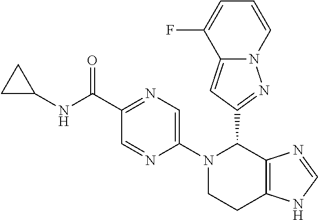 [SMILES: O=C(NC1CC1)c1cnc(N2CCc3[nH]cnc3[C@H]2c2cc3c(F)cccn3n2)cn1] | >100 | 53.5 | 29.8 | >100 | >100 |
| Example A | | | | | |
| 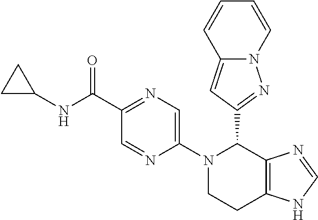 [SMILES: O=C(NC1CC1)c1cnc(N2CCc3[nH]cnc3[C@H]2c2cc3ccccn3n2)cn1] | >100 | >100 | >100 | 97.9 | >100 |
| Example B | | | | | |
| 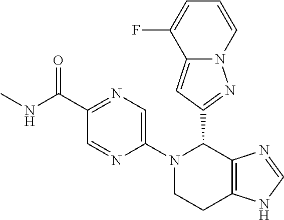 [SMILES: CNC(=O)c1cnc(N2CCc3[nH]cnc3[C@H]2c2cc3c(F)cccn3n2)cn1] | >100 | 75.5 | 41.3 | >100 | >100 |
| Example C | | | | | |
| 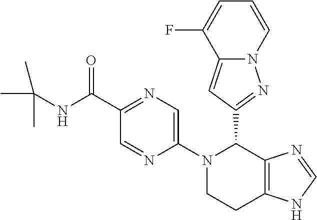 [SMILES: CC(C)(C)NC(=O)c1cnc(N2CCc3[nH]cnc3[C@H]2c2cc3c(F)cccn3n2)cn1] | 53.1 | 20.7 | 3.7 | 54.8 | >100 |
| Example D | | | | | |
| 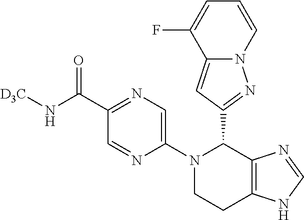 [SMILES: [2H]C([2H])([2H])NC(=O)c1cnc(N2CCc3[nH]cnc3[C@H]2c2cc3c(F)cccn3n2)cn1] | >100 | 52.8 | 35.2 | >100 | >100 |
| Example E | | | | | |
| 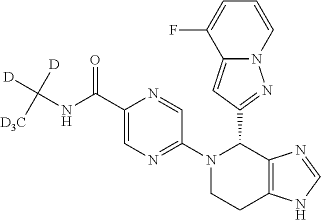 [SMILES: [2H]C([2H])([2H])C([2H])([2H])NC(=O)c1cnc(N2CCc3[nH]cnc3[C@H]2c2cc3c(F)cccn3n2)cn1] | >100 | 36.7 | 29.1 | >100 | >100 |
| Example F | | | | | |
| 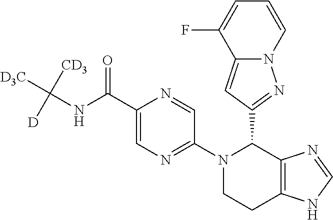 [SMILES: [2H]C([2H])([2H])C([2H])(NC(=O)c1cnc(N2CCc3[nH]cnc3[C@H]2c2cc3c(F)cccn3n2)cn1)C([2H])([2H])[2H]] | 83.9 | 30.5 | 20.7 | >100 | >100 |
| Example G | | | | | |
| 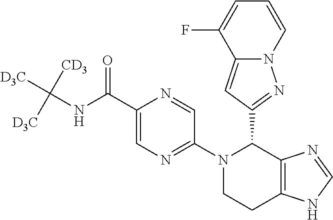 [SMILES: [2H]C([2H])([2H])C(NC(=O)c1cnc(N2CCc3[nH]cnc3[C@H]2c2cc3c(F)cccn3n2)cn1)(C([2H])([2H])[2H])C([2H])([2H])[2H]] | 59.3 | 21.1 | 3.3 | 57 | >100 |
| Example H | | | | | |
| 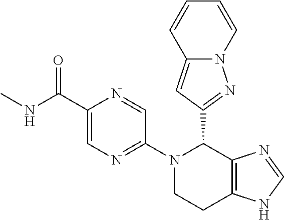 [SMILES: CNC(=O)c1cnc(N2CCc3[nH]cnc3[C@H]2c2cc3ccccn3n2)cn1] | >100 | >100 | 66.9 | >100 | >100 |
| Example I | | | | | |
| 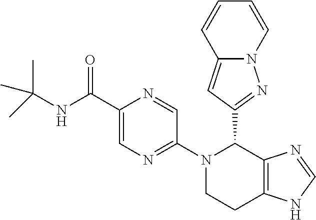 [SMILES: CC(C)(C)NC(=O)c1cnc(N2CCc3[nH]cnc3[C@H]2c2cc3ccccn3n2)cn1] | 88.4 | 29.8 | 7 | >100 | >100 |
| Example J | | | | | |
| 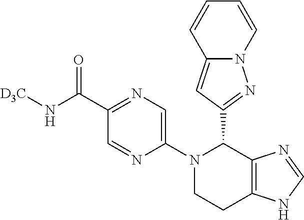 [SMILES: [2H]C([2H])([2H])NC(=O)c1cnc(N2CCc3[nH]cnc3[C@H]2c2cc3ccccn3n2)cn1] | >100 | >100 | >100 | >100 | >100 |
| Example K | | | | | |
| 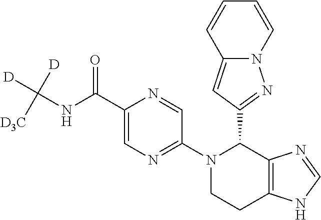 [SMILES: [2H]C([2H])([2H])C([2H])([2H])NC(=O)c1cnc(N2CCc3[nH]cnc3[C@H]2c2cc3ccccn3n2)cn1] | >100 | 49.5 | 45.4 | >100 | >100 |
| Example L | | | | | |
| 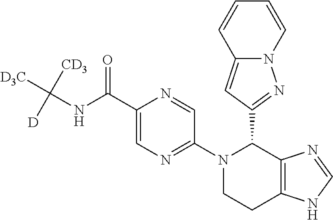 [SMILES: [2H]C([2H])([2H])C([2H])(NC(=O)c1cnc(N2CCc3[nH]cnc3[C@H]2c2cc3ccccn3n2)cn1)C([2H])([2H])[2H]] | >100 | 37.2 | 26.0 | >100 | >100 |
| Example M | | | | | |
| 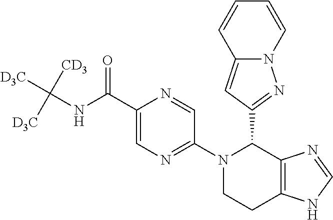 [SMILES: [2H]C([2H])([2H])C(NC(=O)c1cnc(N2CCc3[nH]cnc3[C@H]2c2cc3ccccn3n2)cn1)(C([2H])([2H])[2H])C([2H])([2H])[2H]] | 78.5 | 27.5 | 5.81 | >100 | >100 |
| Example N | | | | | |

In certain aspects, it was found that a hydrogen or fluoro atom at the position corresponding to R¹ in Formula I, provided compounds having improvements in one or more properties of greater stabilization of a mutant PAH protein, greater metabolic stability, greater cellular permeability, greater oral bioavailability, less hERG inhibition potential, less aromatase inhibition potential, and less cytochrome P450 (CYP) inhibition potential when compared to other related compounds. For example, both Examples A and B showed, among other features, binding kinetics that favor stabilization of the mutant PAH protein instead of inhibition (Koff(1/s)), less CYP inhibition potential, and less hERG inhibition potential over Comparators 1 and 2. In addition, Example A had better clearance (Eₕ of <0.29) and greater rat oral bioavailability (54.9% F) than its methyl counterpart Comparator 2 (Eₕ of 0.46; rat oral bioavailability of 19.70% F), and also showed less aromatase inhibition (>30 μM) than its chloro counterpart Comparator 1 (9.45 μM). This and other data are shown below in Table 5.

TABLE 5

| | | 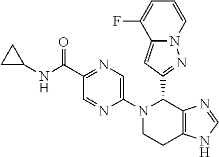 [SMILES: O=C(NC1CC1)c1cnc(N2CCc3[nH]cnc3[C@H]2c2cc3c(F)cccn3n2)cn1] | 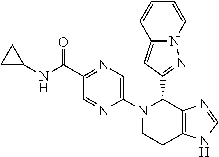 [SMILES: O=C(NC1CC1)c1cnc(N2CCc3[nH]cnc3[C@H]2c2cc3ccccn3n2)cn1] | 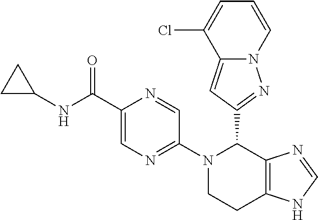 [SMILES: O=C(NC1CC1)c1cnc(N2CCc3[nH]cnc3[C@H]2c2cc3c(Cl)cccn3n2)cn1] | 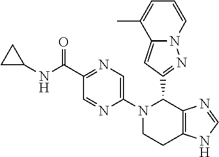 [SMILES: Cc1cccn2nc([C@@H]3c4nc[nH]c4CCN3c3cnc(C(=O)NC4CC4)cn3)cc12] |
|---|---|---|---|---|---|
| | | Example A | Example B | Comparator 1 | Comparator 2 |
| Phe Flux Assay | | 0.148 | 0.348 | 0.099 | 0.161 |
| AC₅₀ (μM) | | | | | |
| Max. ¹³C₉¹⁵N- | | 23.4 | 25.3 | 21.0 | 22.5 |
| Tyrosine Conc. | | | | | |
| (μM) | | | | | |
| SPR K_{D} (μM) / | | 0.487 / 0.028 | 1.15 / 0.032 | 0.027 / 0.00321 | 0.184 / 0.00631 |
| K_{off} (1/s) | | | | | |
| Eₕ | | <0.29 | <0.29 | <0.29 | 0.46 |
| CYP | 1A2 | >100 | >100 | 33.8 | >100 |
| IC₅₀ | (μM) | | | | |
| | 2C19 | 53.5 | >100 | 11.3 | 23 |
| | (μM) | | | | |
| | 2C9 | 29.8 | >100 | 6.39 | 16 |
| | (μM) | | | | |
| | 2D6 | >100 | 97.9 | 44.6 | >100 |
| | (μM) | | | | |
| | 3A4 | >100 | >100 | 82.0 | >100 |
| | (μM) | | | | |

| | | | | |
|---|---|---|---|---|
| Aromatase IC₅₀ | >30 | 26.3 | 9.45 | >30 |
| (μM) | | | | |
| hERG IC₅₀ (μM) | 35.0 | 89.8 | 18.6 | 29.2 |
| Rat | 54.9 | 30.0 | 48.0 | 19.7 |
| Bioavailability | | | | |
| (% F) | | | | |

In certain aspects, it was also found that the presence of a pyrazine-C(O)NH-cyclopropyl moiety (i.e., a cyclopropyl group at R²) in the compounds of the disclosure leads to significant enhancements in one or more of mutant R408W protein binding kinetics, clearance, cellular permeability, decreased aromatase inhibition potential, decreased hERG inhibition potential, and decreased CYP inhibition potential when compared to compounds that do not contain this pyrazine-C(O)NHI-cyclopropyl moiety (i.e., that do not contain cyclopropyl at R²). See, e.g., Table 6 below where the mutant R408W protein binding affinity (Phe SPR K_{D} (μM)) and clearance (Eₕ) for Example A were better than Comparator compounds 3 and 4. In addition, Example A had less hERG inhibition, CYP inhibition, and aromatase inhibition than its cyclobutyl (Comparator 3) counterpart. NT=not tested.

TABLE 6

| | | 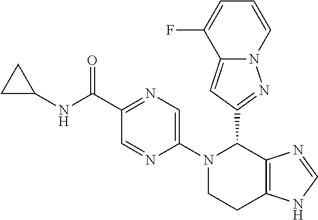 [SMILES: O=C(NC1CC1)c1cnc(N2CCc3[nH]cnc3[C@H]2c2cc3c(F)cccn3n2)cn1] | 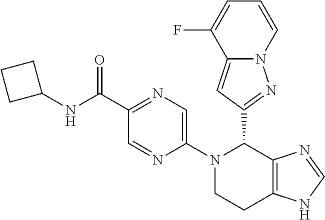 [SMILES: O=C(NC1CCC1)c1cnc(N2CCc3[nH]cnc3[C@H]2c2cc3c(F)cccn3n2)cn1] | 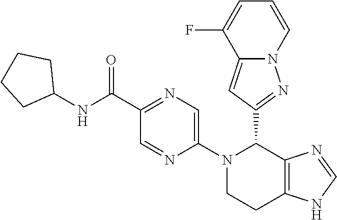 [SMILES: O=C(NC1CCCC1)c1cnc(N2CCc3[nH]cnc3[C@H]2c2cc3c(F)cccn3n2)cn1] |
|---|---|---|---|---|
| | | Example A | Comparator 3 | Comparator 4 |
| Phe Flux Assay | | 0.148 | 0.112 | 0.086 |
| AC₅₀ (μM) | | | | |
| Max. ¹³C₉¹⁵N- | | 23.4 | 21.0 | 16.0 |
| Tyrosine Conc. | | | | |
| (μM) | | | | |
| SPR K_{D} (UM) / | | 0.487 / 0.028 | 2.35 / 0.085 | 2.04 / 0.069 |
| K_{off} (1/s) | | | | |
| Eₕ | | <0.29 | 0.450 | 0.810 |
| CYP | 1A2 | >100 | 72.1 | 35.3 |
| IC₅₀ | (μM) | | | |
| | 2C19 | 53.5 | 14.4 | 13.7 |
| | (μM) | | | |
| | 2C9 | 29.8 | 2.67 | 1.7 |
| | (μM) | | | |
| | 2D6 | >100 | 42.9 | 34.2 |
| | (μM) | | | |
| | 3A4 | >100 | 99.3 | 89.0 |
| | (μM) | | | |

| | | | |
|---|---|---|---|
| Aromatase IC₅₀ | >30 | 21.0 | NT |
| (μM) | | | |
| QPatch hERG | 35.0 | 12.6 | NT |
| IC₅₀ (μM) | | | |

In addition, as shown in Table 7, Example A, which contains a pyrazine moiety substituted by C(O)NH-cyclopropyl, as compared to Comparator 5 (which contains a pyrazine moiety that is not substituted), demonstrated better R408W mutant protein stabilization (lower AC₅₀ and higher max labelled tyrosine concentration), lower clearance, less hERG inhibition potential, and less aromatase inhibition potential. Similarly, Example B, which contains a pyrazine moiety substituted by C(O)NH-cyclopropyl, as compared to Comparator 6 (which contains a pyrazine moiety that is not substituted) demonstrated better R408W mutant protein stabilization (lower AC₅₀ and higher max labelled tyrosine concentration), lower clearance, less hERG inhibition potential, and less aromatase inhibition potential. This and other data are shown below in Table 7.

TABLE 7

| | | 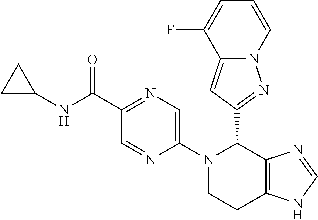 [SMILES: O=C(NC1CC1)c1cnc(N2CCc3[nH]cnc3[C@H]2c2cc3c(F)cccn3n2)cn1] | 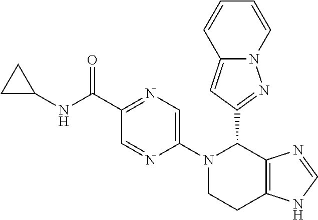 | 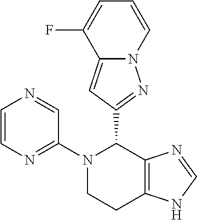 [SMILES: Fc1cccn2nc([C@@H]3c4nc[nH]c4CCN3c3cnccn3)cc12] | 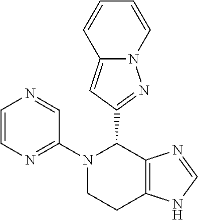 [SMILES: c1ccn2nc([C@@H]3c4nc[nH]c4CCN3c3cnccn3)cc2c1] |
|---|---|---|---|---|---|
| | | Example A | Example B | Comparator 5 | Comparator 6 |
| Phe Flux Assay | | 0.148 | 0.348 | 0.317 | 0.740 |
| AC₅₀ (μM) | | | | | |
| Max. ¹³C₉¹⁵N- | | 23.4 | 25.3 | 21.5 | 17.0 |
| Tyrosine Conc. | | | | | |
| (μM) | | | | | |
| SPR K_{D} (uM) / | | 0.487 / 0.028 | 1.15 /0.032 | 0.030 / 0.021 | 0.078 / 0.029 |
| K_{off} (1/s) | | | | | |
| Eₕ | | <0.29 | <0.29 | 0.490 | 0.400 |
| CYP | 1A2 | >100 | >100 | >100 | >100 |
| IC₅₀ | (μM) | | | | |
| | 2C19 | 53.5 | >100 | 13.8 | 35.4 |
| | (μM) | | | | |
| | 2C9 | 29.8 | >100 | 54.7 | >100 |
| | (μM) | | | | |
| | 2D6 | >100 | 97.9 | >100 | >100 |
| | (μM) | | | | |
| | 3A4 | >100 | >100 | 21.9 | 10.1 |
| | (μM) | | | | |

| | | | | |
|---|---|---|---|---|
| Aromatase IC₅₀ | >30 | 26.3 | 5.85 | 3.91 |
| (μM) | | | | |
| hERG IC₅₀ (μM) | 35.0 | 89.8 | 25.3 | 24.0 |

In certain aspects, it was also found that the presence of a pyrazine-C(O)NHI-methyl moiety or a pyrazine-C(O)NH-t-butyl moiety (i.e., methyl or t-butyl group at R²) in the compounds of the disclosure leads to significant enhancements in one or more of metabolic stability, decreased aromatase inhibition potential, decreased hERG inhibition potential, and decreased CYP inhibition potential when compared to compounds that do not contain this pyrazine-C(O)NH-methyl or pyrazine-C(O)NH-t-butyl moiety. For example, methyl analogue Example C demonstrated less hERG inhibition and better CYP inhibition potential for CYP 2C19 and CYP 2C9 than the isopropyl (Comparator 7), ethyl (Comparator 8), butyl (Comparator 9), and sec-butyl (Comparator 10) counterparts. Example C also demonstrated less aromatase inhibition than Comparators 7, 9, and 10, and had lower clearance than Comparators 9 and 10.

Example D, the t-Butyl analogue, showed superior hERG inhibition potential over the corresponding isopropyl, ethyl, butyl, and sec-butyl derivatives Comparators 5-9. Further, Example D, the t-butyl analogue, had improved clearance (Eₕ) over butyl Comparator 9, and sec-butyl Comparator 10. This, and other data is provided in Tables 8 and 9 below.

TABLE 8

| | | 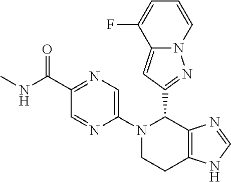 [SMILES: CNC(=O)c1cnc(N2CCc3[nH]cnc3[C@H]2c2cc3c(F)cccn3n2)cn1] | 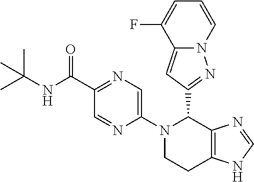 [SMILES: CC(C)(C)NC(=O)c1cnc(N2CCc3[nH]cnc3[C@H]2c2cc3c(F)cccn3n2)cn1] | 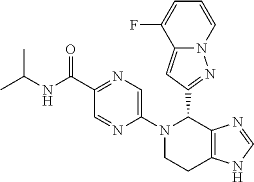 [SMILES: CC(C)NC(=O)c1cnc(N2CCc3[nH]cnc3[C@H]2c2cc3c(F)cccn3n2)cn1] | 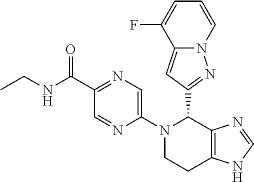 [SMILES: CCNC(=O)c1cnc(N2CCc3[nH]cnc3[C@H]2c2cc3c(F)cccn3n2)cn1] |
|---|---|---|---|---|---|
| | | Example C | Example D | Comparator 7 | Comparator 8 |
| Phe Flux Assay | | 0.444 | 0.088 | 0.167 | 0.136 |
| AC₅₀ (μM) | | | | | |
| Max. ¹³C₉¹⁵N- | | 27.7 | 20.3 | 18.0 | 23.0 |
| Tyrosine Conc. | | | | | |
| (μM) | | | | | |
| SPR K_{D} (μM) / | | 1.15 / 0.071 | 1.272 / 0.022 | 1.67 / 0.059 | 0.992 / 0.054 |
| K_{off} (1/s) | | | | | |
| Eₕ (H) | | <0.29 | <0.29 | <0.29 | <0.29 |
| CYP | 1A2 | >100 | 53.1 | >100 | >100 |
| IC₅₀ | (μM) | | | | |
| | 2C19 | 75.5 | 20.7 | 27.2 | 39.1 |
| | (μM) | | | | |
| | 2C9 | 41.3 | 3.7 | 19.2 | 25.0 |
| | (μM) | | | | |
| | 2D6 | >100 | 54.8 | 90.8 | >100 |
| | (μM) | | | | |
| | 3A4 | >100 | > 100 | >100 | >100 |
| | (μM) | | | | |

| | | | | |
|---|---|---|---|---|
| Aromatase IC₅₀ | >30 | 12.2 | 19.2 | > 30 |
| (μM) | | | | |
| hERG IC₅₀ (μM) | 61.0 | >100 | 35.1 | 18.3 |

TABLE 9

| | | 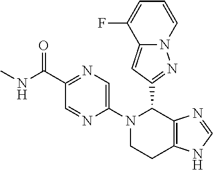 [SMILES: CNC(=O)c1cnc(N2CCc3[nH]cnc3[C@H]2c2cc3c(F)cccn3n2)cn1] | 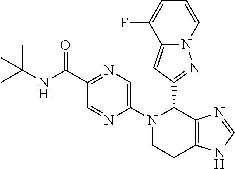 [SMILES: CC(C)(C)NC(=O)c1cnc(N2CCc3[nH]cnc3[C@H]2c2cc3c(F)cccn3n2)cn1] | 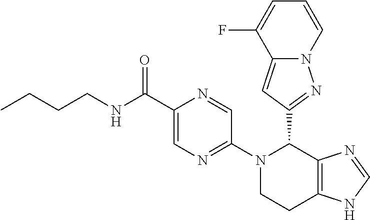 [SMILES: CCCCNC(=O)c1cnc(N2CCc3[nH]cnc3[C@H]2c2cc3c(F)cccn3n2)cn1] | 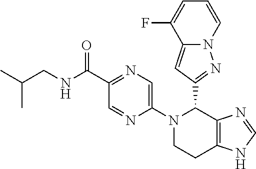 [SMILES: CC(C)CNC(=O)c1cnc(N2CCc3[nH]cnc3[C@H]2c2cc3c(F)cccn3n2)cn1] |
|---|---|---|---|---|---|
| | | Example C | Example D | Comparator 9 | Comparator 10 |
| Phe Flux Assay | | 0.444 | 0.088 | 0.159 | 0.076 |
| AC₅₀ (uM) | | | | | |
| Max. ¹³C₉₁₅N- | | 27.7 | 20.3 | 20.0 | 17.0 |
| Tyrosine Conc. | | | | | |
| (μM) | | | | | |
| SPR K_{D} (μM) / | | 1.15 / 0.071 | 1.272 / 0.022 | 1.867 / 0.072 | 1.581 / 0.056 |
| K_{off} (1/s) | | | | | |
| Eₕ (H) | | <0.29 | <0.29 | 0.580 | 0.730 |
| CYP | 1A2 | >100 | 53.1 | 47.0 | 56.6 |
| IC₅₀ | (μM) | | | | |
| | 2C19 | 75.5 | 20.7 | 11.2 | 15.0 |
| | (μM) | | | | |
| | 2C9 | 41.3 | 3.7 | 1.52 | 2.85 |
| | (μM) | | | | |
| | 2D6 | >100 | 55 | 55.1 | 41.1 |
| | (μM) | | | | |
| | 3A4 | >100 | > 100 | 94.8 | >100 |
| | (μM) | | | | |

| | | | | |
|---|---|---|---|---|
| Aromatase IC₅₀ | >30 | 12.2 | 7.44 | 11.0 |
| (μM) | | | | |
| hERG IC₅₀ (μM) | 61.0 | >100 | 8.21 | 9.15 |

Having now fully described the methods, compounds, and compositions of matter (i.e. pharmaceutical compositions) provided herein, it will be understood by those of skill in the art that the same can be performed within a wide and equivalent range of conditions, formulations, and other parameters without affecting the scope of the methods, compounds, and compositions (i.e. pharmaceutical compositions) provided herein or any embodiment thereof.

All patents, patent applications and publications cited herein are fully incorporated by reference herein in their entirety.

What is claimed is:

1. A compound of Formula I: 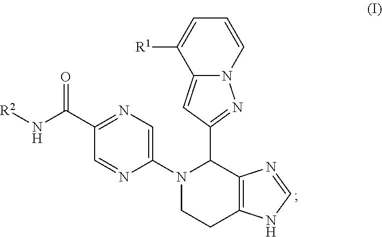
or a tautomer thereof, or a pharmaceutically acceptable salt of the compound or the tautomer, wherein: R¹ is hydrogen or fluoro; and R² is cyclopropyl, methyl, t-butyl, or deuterated(C1-C4)alkyl.

2. The compound of claim 1, wherein the compound is of Formula Ia: 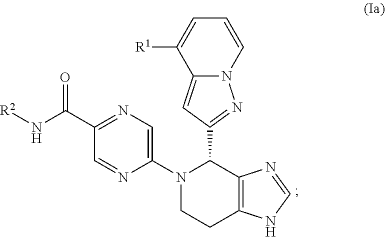
or a tautomer thereof, or a pharmaceutically acceptable salt of the compound or the tautomer.

3. The compound of claim 1, or a tautomer thereof, or a pharmaceutically acceptable salt of the compound or the tautomer, wherein R¹ is hydrogen.

4. The compound of claim 1, or a tautomer thereof, or a pharmaceutically acceptable salt of the compound or the tautomer, wherein R¹ is fluoro.

5. The compound of claim 1, or a tautomer thereof, or a pharmaceutically acceptable salt of the compound or the tautomer, wherein R² is —CD₃, —C(CD₃)₃, —CD₂CD₃, or —CD(CD₃)₂.

6. The compound of claim 1, wherein the compound is 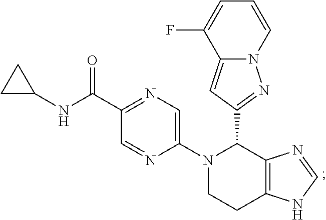 [SMILES: O=C(NC1CC1)c1cnc(N2CCc3[nH]cnc3[C@H]2c2cc3c(F)cccn3n2)cn1]
or a tautomer thereof, or a pharmaceutically acceptable salt of the compound or the tautomer.

7. The compound of claim 1, wherein the compound is 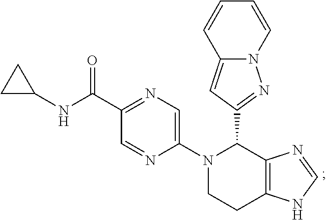 [SMILES: O=C(NC1CC1)c1cnc(N2CCc3[nH]cnc3[C@H]2c2cc3ccccn3n2)cn1]
or a tautomer thereof, or a pharmaceutically acceptable salt of the compound or the tautomer.

8. The compound of claim 1, wherein the compound is 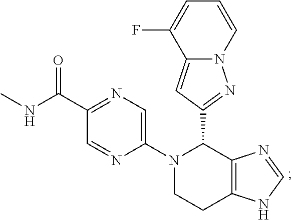 [SMILES: CNC(=O)c1cnc(N2CCc3[nH]cnc3[C@H]2c2cc3c(F)cccn3n2)cn1]
or a tautomer thereof, or a pharmaceutically acceptable salt of the compound or the tautomer.

9. The compound of claim 1, wherein the compound is 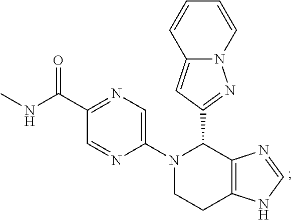 [SMILES: CNC(=O)c1cnc(N2CCc3[nH]cnc3[C@H]2c2cc3ccccn3n2)cn1]
or a tautomer thereof, or a pharmaceutically acceptable salt of the compound or the tautomer.

10. The compound of claim 1, wherein the compound is 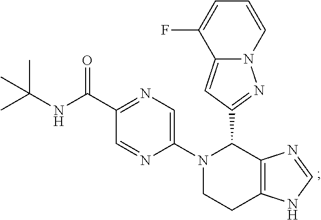 [SMILES: CC(C)(C)NC(=O)c1cnc(N2CCc3[nH]cnc3[C@H]2c2cc3c(F)cccn3n2)cn1]
or a tautomer thereof, or a pharmaceutically acceptable salt of the compound or the tautomer.

11. The compound of claim 1, wherein the compound is 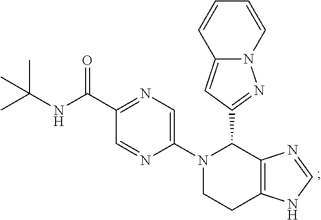 [SMILES: CC(C)(C)NC(=O)c1cnc(N2CCc3[nH]cnc3[C@H]2c2cc3ccccn3n2)cn1]
or a tautomer thereof, or a pharmaceutically acceptable salt of the compound or the tautomer.

12. The compound of claim 1, wherein the compound is 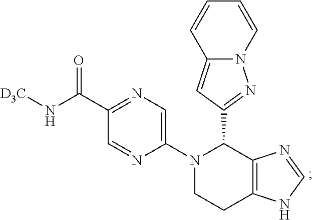 [SMILES: [2H]C([2H])([2H])NC(=O)c1cnc(N2CCc3[nH]cnc3[C@H]2c2cc3ccccn3n2)cn1]
or a tautomer thereof, or a pharmaceutically acceptable salt of the compound or the tautomer.

13. The compound of claim 1, wherein the compound is 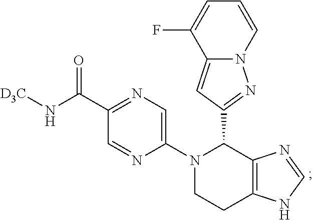 [SMILES: [2H]C([2H])([2H])NC(=O)c1cnc(N2CCc3[nH]cnc3[C@H]2c2cc3c(F)cccn3n2)cn1]
or a tautomer thereof, or a pharmaceutically acceptable salt of the compound or the tautomer.

14. The compound of claim 1, wherein the compound is 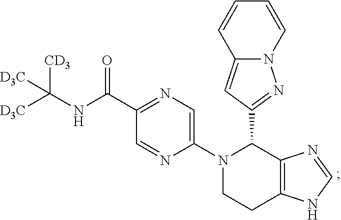 [SMILES: [2H]C([2H])([2H])C(NC(=O)c1cnc(N2CCc3[nH]cnc3[C@H]2c2cc3ccccn3n2)cn1)(C([2H])([2H])[2H])C([2H])([2H])[2H]]
or a tautomer thereof, or a pharmaceutically acceptable salt of the compound or the tautomer.

15. The compound of claim 1, wherein the compound is 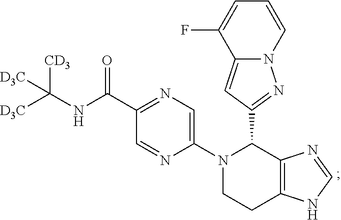 [SMILES: [2H]C([2H])([2H])C(NC(=O)c1cnc(N2CCc3[nH]cnc3[C@H]2c2cc3c(F)cccn3n2)cn1)(C([2H])([2H])[2H])C([2H])([2H])[2H]]
or a tautomer thereof, or a pharmaceutically acceptable salt of the compound or the tautomer.

16. The compound of claim 1, wherein the compound is 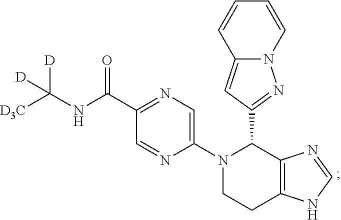 [SMILES: [2H]C([2H])([2H])C([2H])([2H])NC(=O)c1cnc(N2CCc3[nH]cnc3[C@H]2c2cc3ccccn3n2)cn1]
or a tautomer thereof, or a pharmaceutically acceptable salt of the compound or the tautomer.

17. The compound of claim 1, wherein the compound is 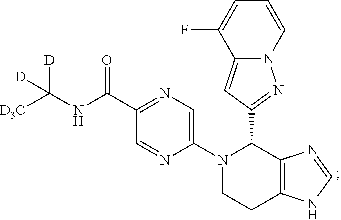 [SMILES: [2H]C([2H])([2H])C([2H])([2H])NC(=O)c1cnc(N2CCc3[nH]cnc3[C@H]2c2cc3c(F)cccn3n2)cn1]
or a tautomer thereof, or a pharmaceutically acceptable salt of the compound or the tautomer.

18. The compound of claim 1, wherein the compound is 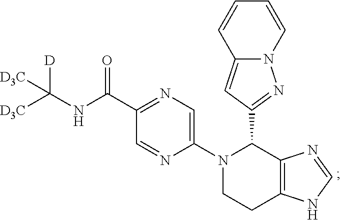 [SMILES: [2H]C([2H])([2H])C([2H])(NC(=O)c1cnc(N2CCc3[nH]cnc3[C@H]2c2cc3ccccn3n2)cn1)C([2H])([2H])[2H]]
or a tautomer thereof, or a pharmaceutically acceptable salt of the compound or the tautomer.

19. The compound of claim 1, wherein the compound is 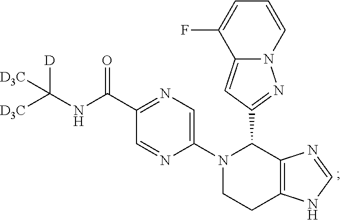 [SMILES: [2H]C([2H])([2H])C([2H])(NC(=O)c1cnc(N2CCc3[nH]cnc3[C@H]2c2cc3c(F)cccn3n2)cn1)C([2H])([2H])[2H]]
or a tautomer thereof, or a pharmaceutically acceptable salt of the compound or the tautomer.

20. A pharmaceutical composition comprising a compound of claim 1, or a tautomer thereof, or a pharmaceutically acceptable salt of the compound or the tautomer; and a pharmaceutically acceptable excipient.

21. A pharmaceutical composition comprising a compound of claim 2, or a tautomer thereof, or a pharmaceutically acceptable salt of the compound or the tautomer, and a pharmaceutically acceptable excipient.

22. A pharmaceutical composition comprising a compound of claim 5, or a tautomer thereof, or a pharmaceutically acceptable salt of the compound or the tautomer, and a pharmaceutically acceptable excipient.

23. A pharmaceutical composition comprising a compound of claim 6, or a tautomer thereof, or a pharmaceutically acceptable salt of the compound or the tautomer, and a pharmaceutically acceptable excipient.

24. A pharmaceutical composition comprising a compound of claim 7, or a tautomer thereof, or a pharmaceutically acceptable salt of the compound or the tautomer, and a pharmaceutically acceptable excipient.

25. A pharmaceutical composition comprising a compound of claim 8, or a tautomer thereof, or a pharmaceutically acceptable salt of the compound or the tautomer, and a pharmaceutically acceptable excipient.

26. A pharmaceutical composition comprising a compound of claim 9, or a tautomer thereof, or a pharmaceutically acceptable salt of the compound or the tautomer, and a pharmaceutically acceptable excipient.

27. A pharmaceutical composition comprising a compound of claim 10, or a tautomer thereof, or a pharmaceutically acceptable salt of the compound or the tautomer, and a pharmaceutically acceptable excipient.

28. A pharmaceutical composition comprising a compound of claim 11, or a tautomer thereof, or a pharmaceutically acceptable salt of the compound or the tautomer, and a pharmaceutically acceptable excipient.


SEQUENCE LISTING (2 sequences)

SEQ ID NO: 1 - protein, 7 aa, synthetic construct
ENLYFQG

SEQ ID NO: 2 - protein, 17 aa, synthetic construct
GLNDIFEQQKIEWHEGS